(19) United States
(12) Patent Application Publication
(10) Pub. No.: US 2023/0242992 A1
(43) Pub. Date: Aug. 3, 2023

(54) METHODS OF PREDICTING CANCER PROGRESSION

(71) Applicant: GMDx Co Pty Ltd, Camberwell Victoria (AU)

(72) Inventors: Robyn A. LINDLEY, Camberwell (AU); Nathan E. HALL, Camberwell (AU); Jared MAMROT, Camberwell (AU)

(21) Appl. No.: 17/928,784

(22) PCT Filed: Jun. 1, 2021
(86) PCT No.: PCT/AU21/50535

§ 371 Date: Nov. 30, 2022

(30) Foreign Application Priority Data
Jun. 1, 2020 (AU) 2020901790

(51) Int. Cl. C12Q 1/6886; C12Q 1/6869; G16B 20/20; G16H 50/20; G16H 50/30; G16H 50/70

(52) U.S. Cl. CPC C12Q 1/6886; C12Q 1/6869; G16B 20/20; G16H 50/20; G16H 50/30; G16H 50/70; C12Q 2600/112; C12Q 2600/118

(57) ABSTRACT

This invention relates generally to systems and methods of predicting the likelihood of cancer progression or recurrence. More particularly, the present invention relates to systems and methods of identifying nucleic acid mutation signatures that correlate with the likelihood of cancer recurrence or progression, and methods of using such signatures.

This application claims priority to Australian Provisional Application No. 2020901790 entitled “Methods of predicting cancer progression” filed 1 Jun. 2020, the contents of which are incorporated herein by reference in their entirety.

### FIELD OF THE INVENTION

This invention relates generally to systems and methods of predicting the likelihood of cancer progression or recurrence. More particularly, the present invention relates to systems and methods of identifying nucleic acid mutation signatures that correlate with the likelihood of cancer recurrence or progression, and methods of using such signatures.

### BACKGROUND OF THE INVENTION

Accurately predicting the likelihood of cancer progression and/or recurrence is an important step in developing appropriate treatment protocols, including which therapeutic agents to administer to a particular patient, when to administer them and at what dose to administer them. To this end, various studies have been performed to identify genetic signatures associated with cancer progression (referred to herein as cancer progression associated signatures, or CPAS). Many of these studies can be considered to be based on gene-centric approach, with the identification of single nucleotide polymorphisms (SNPs) as prognostic markers.

The results of many of these studies are curated in the COSMIC database. For example, a major study by Li M et al (2017) has identified variants on several known oncogenes that include for example variants on the FOXM1, E2F1 and PYGM genes that are found to be related to progression in many different cancer types that include bladder carcinoma, adenocarcinoma, Ewing's sarcoma, germ cell tumours, malignant melanoma and breast cancer.

In many instances to date, the identified cancer progression prediction markers are single variant (or a combination of single variants) genetic biomarkers, and as such each is only found in a small proportion of the cancer patient population (i.e. 1%-5%). The utility of these in a heterogenous population may therefore be limited. Moreover, the markers do not provide an indication of the likely source of the variation or mutation, knowledge that can be of benefit for the development of future diagnostics and therapeutics.

There remains a need for the identification of additional cancer progression associated signatures and the development of further methods for determining the likelihood of cancer progression and/or recurrence in a patient with cancer.

### SUMMARY OF THE INVENTION

The present invention is predicated in part on the identification of genetic signatures associated with cancer progression (referred to herein as cancer progression associated signatures, or CPAS), and methods for predicting or determining the likelihood or probability of cancer progression and/or recurrence in a patient with cancer. Accordingly, one advantage of this method is that it allows for a treatment regimen for a subject who has or has had a cancer, to be prescribed based on the determination of the likelihood that the cancer will progress or recur. For example, if a cancer is determined as being likely to progress or recur in a subject, the subject may continue a heavy course of anti-cancer therapy or may be administered a more aggressive course of anti-cancer therapy. Conversely, if a cancer is determined to be unlikely to recur in a subject, the subject may discontinue, reduce, or change an existing anti-cancer therapy.

Thus, in one aspect, provided is a method for determining the likelihood that a cancer in a subject will progress or recur, the method comprising: analyzing the sequence of a nucleic acid molecule from a subject with cancer to detect single nucleotide variations (SNVs) within the nucleic acid molecule; determining a plurality of metrics based on the number and/or type of SNVs detected so as to obtain a subject profile of metrics; and, determining the likelihood that the cancer will progress or recur based on a comparison between the subject profile and a reference profile of metrics; wherein the plurality of metrics comprises 5 or more metrics (e.g. at least 5, 10, 15, 20, 35, 30, 40, 45 or 50 metrics) selected from the metrics set forth in Table D and metrics related to the metrics set forth in Table D. In some examples, the reference profile is representative of a cancer that is likely to progress or recur. In other examples, the reference profile is representative of a cancer (or subject with a cancer) that is unlikely to progress or recur.

Also provided is a method for treating a subject with cancer, comprising exposing to the subject a cancer therapy on the basis of a determination that the cancer or tumour is likely to progress or recur according to the method described above and herein.

In another aspect, provided is a method of treating a cancer in a subject, the method comprising: (i) performing the method for determining the likelihood that a cancer in a subject will progress or recur as described above and herein; (ii) determining that the cancer is likely to progress or recur; and (iii) exposing the subject to a cancer therapy (e.g. radiotherapy, surgery, chemotherapy, hormone therapy, immunotherapy or targeted therapy).

In a further aspect, provided is a system for generating a progression indicator for use in assessing the likelihood of cancer progression or recurrence in a subject, the system including one or more electronic processing devices that: a) obtain subject data indicative of a sequence of a nucleic acid molecule from the subject; b) analyze the subject data to identify single nucleotide variations (SNVs) within the nucleic acid molecule; c) determine a plurality of metrics using the identified SNVs, the plurality of metrics including 5 or more metrics (e.g. at least 5, 10, 15, 20, 35, 30, 40, 45 or 50 metrics) selected from the metrics set forth in Table D and metrics related to the metrics set forth in Table D; d) apply the plurality of metrics to at least one computational model to determine a progression indicator indicative of a likelihood of progression or recurrence of a cancer, the at least one computational model embodying a relationship between a likelihood of progression or recurrence of a cancer and the plurality of metrics and being derived by applying machine learning to a plurality of reference metrics obtained from reference subjects having a known progression or recurrence of a cancer. In some examples, the at least one computational model includes a decision tree. In a particular example, the at least one computational model includes a plurality of decision trees, and the therapy indicator is generated by aggregating results from the plurality of decision trees.

In another aspect, provided is a system for use in calculating at least one computational model, the at least one computational model being used for generating a progression indicator for use in assessing likelihood of cancer progression or recurrence in a subject, the system including one or more electronic processing devices that: a) for each of a plurality of reference subjects: i) obtain reference subject data indicative of: (1) a sequence of a nucleic acid molecule from the reference subject; and, (2) progression or recurrence of cancer; ii) analyze the reference subject data to identify single nucleotide variations (SNVs) within the nucleic acid molecule; iii) determine a plurality of metrics using the identified SNVs, the plurality of metrics including 5 or more metrics (e.g. at least 5, 10, 15, 20, 35, 30, 40, 45 or 50 metrics) selected from the metrics set forth in Table D and metrics related to the metrics set forth in Table D; and, b) use the plurality of reference metrics and known progression or recurrence of cancer of reference subjects to train at least one computational model, the at least one computational model embodying a relationship between progression or recurrence of cancer and the plurality of metrics.

In some embodiments of such a system, the one or more processing devices test the at least one computational model to determine a discriminatory performance of the model. In some examples, the discriminatory performance is based on at least one of: a) an area under a receiver operating characteristic curve; b) an accuracy; c) a sensitivity; and, d) a specificity. In one example, the discriminatory performance is at least 60%.

In some embodiments, the one or more processing devices test the at least one computational model using a reference subject data from a subset of the plurality of reference subjects. In some embodiments, the one or more processing devices: a) select a plurality of reference metrics; b) train at least one computational model using the plurality of reference metrics; c) test the at least one computational model to determine a discriminatory performance of the model; and, d) if the discriminatory performance of the model falls below a threshold, at least one of: i) selectively retrain the at least one computational model using a different plurality of reference metrics; and, ii) train a different computational model. In further embodiments, the one or more processing devices: a) select a plurality of combinations of reference metrics; b) train a plurality of computational models using each of the combinations; c) test each computational model to determine a discriminatory performance of the model; and, d) selecting the at least one computational model with the highest discriminatory performance for use in determining the progression indicator.

In another aspect, provided is a method for generating a progression indicator for use in assessing likelihood of cancer progression or recurrence in a subject, the method including, in one or more electronic processing devices: a) obtaining subject data indicative of a sequence of a nucleic acid molecule from the subject; b) analyzing the subject data to identify single nucleotide variations (SNVs) within the nucleic acid molecule; c) determining a plurality of metrics using the identified SNVs, the plurality of metrics including 5 or more metrics (e.g. at least 5, 10, 15, 20, 35, 30, 40, 45 or 50 metrics) selected from the metrics set forth in Table D and metrics related to the metrics set forth in Table D; and, d) applying the plurality of metrics to at least one computational model to determine a progression indicator indicative of progression or recurrence of cancer, the at least one computational model embodying a relationship between progression or recurrence of cancer and the plurality of metrics and being derived by applying machine learning to a plurality of reference metrics obtained from reference subjects having a known progression or recurrence of cancer.

In some embodiments of the methods and systems of the present disclosure, the cancer is selected from among adrenal cancer, breast cancer, brain cancer, prostate cancer, liver cancer, colon cancer, stomach cancer, pancreatic cancer, skin cancer, thyroid, cervical cancer, lymphoid cancer, hematopoietic cancer, bladder cancer, lung cancer, renal cancer, rectal cancer, ovarian cancer, uterine cancer, head and neck cancer, mesothelioma and sarcoma.

In particular embodiments, the cancer is mesothelioma and the plurality of metrics comprises least or about 5 metrics selected from cds:A3Bf_ST-C-G Ti %; g:3Gen2_T-C-G C>T+G>A g %; cds:2Gen1_-C-C C>T at MC1%; cds:All C Ti/Tv %; g:3Gen3_CA-C-C>T+G>A g %; cds:3Gen2_C-C-C MC3%; cds:A3Gn_YYC-C-S C>T %; cds:A3G_C-C-MC3%; cds:3Gen3_GG-C-non-syn g:3Gen2_A-C-C C>A+G>T g cds:4Gen3_TT-C-C cds:3Gen2_C-C-T MC3%; g:2Gen1_-C-T C>G+G>C g %; cds:Primary Deaminase %; cds:A3Gb_-C-G G>A at MC2 motif %; cds:4Gen3_CA-C-C %; cds:A3G_C-C-G>T %; cds:A3Gi_SG-C-G non-syn %; g:C>G+G>C %; cds:Other MC3%; cds:A3B_T-C-W G>A motif %, and related metrics thereto.

In other embodiments, the cancer is adrenocortical carcinoma and the plurality of metrics comprises least or about 5 metrics selected from cds:All G total; cds:3Gen1_-C-TG G non-syn %; g:A3F_T-C-Hits; cds:3Gen3_GG-C-non-syn %; cds:3Gen1_-C-GT G>A motif %; cds:A3Bj_RT-C-G Ti %; cds:3Gen2_C-C-T MC3%; nc:A3G_C-C-C>T+G>A nc %; cds:AIDd_WR-C-Y %; cds:3Gen1_-C-TC C>T cds %; cds:A3B_T-C-W G>A motif %; g:CG total; cds:A3G_C-C-MC3%; cds:AIDb_WR-C-G G non-syn %; cds:A3G_C-C-C>T at MC1%; cds:3Gen3_TG-C-G>A %; g:3Gen3_GA-C-C>A+G>T g %; cds:3Gen2_A-C-G MC2 non-syn %; cds:3Gen3_CT-C-MC3%; cds:ADAR_2 Gen2_G-T-MC2%; cds:ADAR_3 Gen3_CA-A-Ti %; g:AIDh_WR-C-T C>A+G>T g %; cds:A3B_T-C-W MC3 non-syn %; cds:2Gen1_-C-C C>A %; cds:A1_-C-A G>A at MC3 cds %; cds:3Gen1_-C-CA Ti C:G %; cds:ADAR_W-A-non-syn %; cds:3Gen1_-C-CA Ti %; cds:All G %; g:3Gen2_T-C-G C>T+G>A g %; cds:A3Gb_-C-G MC1%; cds:A3B_T-C-W G non-syn %; nc:2Gen2_A-C-C>T+G>A nc %; cds:A3Gi_SG-C-G non-syn %; cds:Other G MC3 Ti/Tv %; cds:A3Gb_-C-G G>A at MC2 motif %; cds:A3B_T-C-W Ti %; and g:2Gen1_-C-T %, and related metrics thereto.

In further embodiments, the cancer is brain cancer and the plurality of metrics comprises least or about 5 metrics selected from g:CG total; cds:AIDd_WR-C-Y %; variants in VCF; cds:4Gen3_TA-C-C non-syn %; cds:3Gen2_C-C-T MC3%; cds:AIDd_WR-C-Y G>C %; cds:A3Gb_-C-G MC1%; g:3Gen2_T-C-G C>T+G>A g %; cds:A3B_T-C-W G non-syn %; g:3Gen3_GA-C-C>A+G>T g %; cds:2Gen2_G-C-Hits; cds:AIDc_WR-C-GS MC3%; cds:All G total; cds:All A non-syn %; cds:ADAR_2 Gen2_T-T-%; cds:3Gen2_A-C-C non-syn %; g:3Gen3_CA-C-C>T+G>A g %; g:ADARk_CW-A-A>G+T>C g %; nc:ADARb_W-A-Y A>G+T>C nc %; g:2Gen1_-C-T %; cds:Other MC3 C %; g:2Gen1_-C-T C>G+G>C g %; cds:ADAR_W-A-non-syn %; g:3Gen2_A-C-C C>A+G>T g %; g:ADAR_2 Gen2_G-T-A>T+T>A %; cds:A3G_C-C-C>T at MC1%; cds:3Gen1_-C-GC MC2%; cds:3Gen2_G-C-T %; cds:A3F_T-C-G>C %; g:4Gen3_GG-C-G C>T+G>A g %; cds:A3Gb_-C-G G>A at MC2 motif %; cds:ADARb_W-A-Y MC2%; cds:All G %; g:A3F_T-C-Hits; cds:3Gen2_T-C-C MC1%; cds:A3B_T-C-W Ti %; cds:ADAR_3 Gen1_-A-AT Ti %; cds:ADARh_W-A-S T>C %; cds:A3Gn_YYC-C-S C>T %; cds:A3Ge_SC-C-GS %; cds:2Gen2_A-C-MC3%; cds:ADAR_2 Gen2_G-T-MC2%; cds:ADAR_3 Gen3_CA-A-Ti %; cds:Primary Deaminase %; g:C>G+G>C %; cds:A3Bf_ST-C-G Ti %; cds:3Gen3_CT-C-MC3%; cds:A3Gi_SG-C-G non-syn %; cds:Other MC3%; cds:ADAR_3 Gen1_-A-CA %; cds:A3F_T-C-C>A %; cds:2Gen1_-C-C C>T at MC1%; cds:A3Gc_C-C-GW C>T motif %; cds:AIDc_WR-C-GS %; g:ADAR_2 Gen1_-T-T A>T+T>A %; cds:A3B_T-C-W MC1%; cds:ADAR_3 Gen2_G-A-C non-syn %; cds:2Gen1_-C-C C>A %; cds:3Gen1_-C-GT G>A motif %; cds:A3Bj_RT-C-G Ti %; g:3Gen1_-C-TC C>T+G>A g %; g:C>A+G>T %; cds:3Gen2_A-C-C MC2%; cds:2Gen1_-C-C MC2%; g:3Gen2_G-C-T %; g:A3Bj_RT-C-G C>T+G>A g %; g:ADAR_W-A-A>G+T>C %; cds:3Gen3_AT-C-C:G %; cds:3Gen1_-C-TG G non-syn %; cds:Other G MC3 Ti/Tv %; cds:A3Gb_-C-G G>A MC2 Hits; cds:3Gen1_-C-TC C>T cds %; cds:2Gen1_-C-T MC3 non-syn %; cds:AIDb_WR-C-G G non-syn %; g:AIDc_WR-C-GS Hits; cds:3Gen2_T-C-C MC3%; cds:3Gen2_T-C-G Ti/Tv %; cds:A1_-C-A G>A at MC3 cds %; nc:A3G_C-C-C>T+G>A nc %; nc:2Gen2_A-C-C>T+G>A nc %; cds:3Gen3_TG-C-G Ti/Tv %; cds:3Gen1_-C-CA Ti %; cds:3Gen3_TG-C-G>A %; cds:3Gen3_CT-C-G non-syn %; cds:All C Ti/Tv %; cds:A3G_C-C-MC3%; cds:ADARc_SW-A-Y MC2%; and cds:3Gen3_GG-C-non-syn %, and related metrics thereto.

In other embodiments, the cancer is sarcoma and the plurality of metrics comprises least or about 5 metrics selected from cds:Other MC3 C %; nc:ADARb_W-A-Y A>G+T>C nc %; cds:4Gen3_TT-C-T %; g:ADARk_CW-A-A>G+T>C g %; g:ADARn_-A-WA A>G+T>C %; cds:A3G_C-C-G>T %; cds:A3Gb_-C-G MC1%; nc:ADARb_W-A-Y %; cds:A3Ge_SC-C-GS %; cds:Primary Deaminase %; cds:ADAR_2 Gen2_G-T-MC2%; g:4Gen3_GG-C-G C>T+G>A g %; cds:2Gen1_-C-C MC2%; cds:3Gen1_-C-GT G>A motif %; cds:A3Gn_YYC-C-S C>T %; cds:2Gen1_-C-C C>T at MC1%; cds:A3B_T-C-W MC3 non-syn %; cds:AIDd_WR-C-Y %; g:3Gen3_CA-C-C>T+G>A g %; cds:All A non-syn %; g:2Gen1_-C-T C>G+G>C g %; cds:ADARb_W-A-Y MC2%; cds:All G %; g:A3Bj_RT-C-G C>T+G>A g %; cds:A3Gn_YYC-C-S C>T at MC3 cds %; cds:A3B_T-C-W G non-syn %; cds:A3G_C-C-MC3%; cds:All G total; cds:CDS Variants; g:CG total; g:3Gen2_T-C-G C>T+G>A g %; cds:A3B_T-C-W MC1%; cds:ADAR_3 Gen3_CA-A-Ti %; cds:AIDc_WR-C-GS %, and related metrics thereto.

In further embodiments, the cancer is lung cancer and the plurality of metrics comprises least or about 5 metrics selected from cds:3Gen1_-C-CC C>T at MC1 motif %; cds:3Gen1_-C-CT C>T at MC2 cds %; cds:ADARp_-A-WT A>G at MC2 cds %; cds:Other MC3 C %; cds:Other MC3%; cds:A3Gb_-C-G MC1%; g:3Gen1_-C-TC C>T+G>A g %; cds:ADAR_W-A-A>G at MC3%; cds:ADAR_W-A-non-syn %; cds:ADAR_3 Gen3_AC-A-A>G cds %; cds:2Gen1_-C-C C>A %; cds:ADARf_SW-A-MC2%; g:ADAR_2 Gen2_G-T-A>T+T>A %; cds:4Gen3_GC-C-A %; cds:A3Go_TC-C-G MC1 non-syn %; g:3Gen2_G-C-T %; cds:A3G_C-C-C>T at MC1%; cds:AIDc_WR-C-GS MC3%; cds:3Gen1_-C-GT G>A motif %; nc:2Gen1_-C-T C>A+G>T nc %; cds:ADARc_SW-A-Y MC2%; cds:ADARh_W-A-S T>C %; cds:2Gen1_-C-C C>T at MC1%; g:ADAR_2 Gen1_-T-T A>T+T>A %; cds:AIDd_WR-C-Y C>A cds %; nc:A3G_C-C-C>T+G>A nc %; cds:A3Gc_C-C-GW C>T motif %; cds:ADAR_3 Gen1_-A-AT Ti %; cds:3Gen3_CT-C-MC3%; cds:4Gen3_CT-C-C C>T at MC1%; cds:3Gen2_T-C-C MC1%; cds:A3G_C-C-G>T %; cds:3Gen1_-C-CA Ti %; cds:3Gen1_-C-TG G non-syn %; cds:3Gen2_A-C-C non-syn %; g:2Gen1_-C-T C>G+G>C g %; cds:All A non-syn %; cds:A3Gi_SG-C-G MC2%; cds:Primary Deaminase %; cds:4Gen3_TT-C-T %; g:A3Bj_RT-C-G C>T+G>A g %; cds:3Gen2_T-C-C MC3%; cds:4Gen3_TT-C-C %; cds:3Gen1_-C-CA Ti C:G %; cds:A1_-C-A G>A at MC3 cds %; cds:A3Gb_-C-G G>A at MC2 motif %; cds:3Gen3_CT-C-G non-syn %; cds:3Gen2_G-C-T C:G %; cds:A3Ge_SC-C-GS %; cds:3Gen3_TG-C-G>A %; g:C>A+G>T %; cds:4Gen3_CA-C-C %; cds:AIDd_WR-C-Y G>C %; cds:All G %; cds:3Gen3_TT-C-C>A at MC1 motif %; g:AIDh_WR-C-T C>A+G>T g %; g:4Gen3_GG-C-G C>T+G>A g %; cds:3Gen2_G-C-T C>A motif %; nc:ADARc_SW-A-Y A>G+T>C nc %; g:3Gen2_A-C-C C>A+G>T g %; cds:A3B_T-C-W Ti %; g:3Gen3_GA-C-C>A+G>T g %; cds:3Gen3_CT-C-C>T at MC1 motif %; cds:ADAR_3 Gen1_-A-CC A>G cds %; cds:3Gen1_-C-TC C>T cds %; cds:4Gen3_CA-C-C MC1%; cds:3Gen2_G-C-T %; nc:2Gen2_A-C-C>T+G>A nc %; cds:3Gen2_A-C-C MC2%; cds:A3F_T-C-C>A %; cds:CDS Variants; cds:ADAR_3 Gen3_CA-A-Ti %; cds:3Gen3_GG-C-non-syn %; cds:ADARb_W-A-Y MC2%; g:ADAR_W-A-A>G+T>C %; cds:3Gen3_AT-C-C:G %; cds:2Gen1_-C-C G>T at MC1%; cds:A3G_C-C-MC3%; cds:3Gen2_C-C-C MC3%; cds:A3B_T-C-W G>A motif %; cds:A3F_T-C-G>C %; cds:ADAR_2 Gen2_G-T-MC2%; cds:3Gen1_-C-AG G Ti/Tv %; cds:A3Bj_RT-C-G Ti %; nc:ADARb_W-A-Y A>G+T>C nc %; cds:ADAR_2 Gen2_T-T-%; g:2Gen1_-C-T %; cds:4Gen3_AC-C-T Ti/Tv %; cds:A3Gi_SG-C-G non-syn %; cds:A3Bf_ST-C-G Ti %; g:ADARk_CW-A-A>G+T>C g %; cds:3Gen1_-C-GC MC2%; g:3Gen3_CA-C-C>T+G>A g %; cds:2Gen2_A-C-MC3%; variants in VCF; cds:4Gen3_AG-C-T MC1 non-syn %; g:3Gen2_T-C-G C>T+G>A g %; cds:A3Gn_YYC-C-S C>T at MC3 cds %; cds:ADAR_3 Gen1_-A-CA %; cds:4Gen3_TA-C-C non-syn %; cds:All C Ti/Tv %; cds:ADARc_SW-A-Y, and related metrics thereto.

In some embodiments, the cancer is skin cancer and the plurality of metrics comprises least or about 5 metrics selected from cds:4Gen3_AG-C-T MC1 non-syn %; cds:3Gen1_-C-CG G>A at MC3%; cds:4Gen3_AC-C-T Ti/Tv %; g:C>G+G>C %; cds:A3B_T-C-W MC3 non-syn %; cds:All A non-syn %; cds:3Gen3_AG-C-MC2%; cds:A3B_T-C-W MC1%; cds:ADAR_3 Gen2_C-A-C T>G at MC3 cds %; cds:3Gen1_-C-TC C>T at MC3%; cds:4Gen3_GC-C-C C>T at MC2%; cds:All C Ti/Tv %; cds:A3Bj_RT-C-G Ti %; cds:AIDh_WR-C-T G>A at MC2 cds %; cds:4Gen3_TT-C-C %; cds:3Gen1_-C-CC C>T at MC1 motif %; cds:ADAR_2 Gen2_T-T-%; cds:3Gen2_T-C-C MC1%; cds:All G %; cds:ADAR_W-A-A>G at MC3%; cds:A3G_C-C-MC3%; cds:Other MC3 C %; g:3Gen2_A-C-C C>A+G>T g %; cds:ADARc_SW-A-Y MC2%; cds:3Gen1_-C-CA Ti C:G %; cds:3Gen1_-C-TC C>T cds %; cds:3Gen2_C-C-C MC3%; cds:3Gen3_CT-C-C>T at MC1 motif %; g:ADAR_4 Gen3_AG-A-G A>C+T>G %; cds:3Gen3_CT-C-G non-syn %; cds:3Gen2_A-C-C non-syn %; cds:2Gen2_A-C-MC3%; cds:3Gen2_A-C-C MC2%; g:3Gen1_-C-TC C>T+G>A g %; cds:3Gen2_T-C-T G>A at MC2%; cds:2Gen1_-C-C C>T at MC1%; cds:AIDb_WR-C-G G non-syn %; cds:A3Gb_-C-G MC1%; cds:2Gen1_-C-C C>A %; cds:A3Ge_SC-C-GS %; g:ADARn_-A-WA A>G+T>C %; g:ADAR_W-A-A>G+T>C %; g:ADAR_2 Gen2_G-T-A>T+T>A %; g:AIDh_WR-C-T C>A+G>T g %; cds:4Gen3_TG-C-T Ti C:G %; cds:3Gen2_G-C-T C:G %; cds:3Gen2_T-C-C MC3%; nc:ADARb_W-A-Y %; cds:ADAR_3 Gen2_G-A-C non-syn %; cds:ADAR_3 Gen1_-A-AT Ti %; g:ADARk_CW-A-A>G+T>C g %; cds:3Gen1_-C-GC MC2%; cds:4Gen3_TA-C-C non-syn %; g:3Gen3_CA-C-C>T+G>A g %; cds:3Gen1_-C-AG G Ti/Tv %; cds:AIDc_WR-C-GS %; cds:A3Gn_YYC-C-S C>T at MC3 cds %; cds:2Gen1_-C-C MC2%; cds:3Gen3_GG-C-non-syn %; g:2Gen1_-C-T C>G+G>C g %; cds:A1_-C-A G>A at MC3 cds %; cds:A3G_C-C-C>T at MC1%; nc:ADARc_SW-A-Y A>G+T>C nc %; cds:ADAR_W-A-T>C at MC2%; cds:A3Go_TC-C-G MC1 non-syn %; cds:3Gen3_AT-C-C:G %; cds:ADARh_W-A-S T>C %; cds:A3G_C-C-G>T %; cds:ADARf_SW-A-MC2%; cds:ADAR_W-A-non-syn %; cds:ADARp_-A-WT T>A motif %; cds:4Gen3_AG-C-T G>A at MC1 motif %; cds:ADAR_3 Gen1_-A-CA %; cds:3Gen2_C-C-T MC3%; cds:3Gen1_-C-CT C>T at MC2 cds %; cds:A3B_T-C-W Ti %; g:2Gen1_-C-T %; cds:AIDc_WR-C-GS MC3%; cds:AIDe_WR-C-GW Hits; cds:AIDd_WR-C-Y C>A cds %; cds:ADARb_W-A-Y MC2%; cds:A3Gc_C-C-GW C>T motif %; cds:2Gen1_-C-C G>T at MC1%; cds:3Gen1_-C-CA Ti %; cds:Other G MC3 Ti/Tv %; cds:CDS Variants; cds:ADAR_3 Gen1_-A-CC A>G cds %; cds:A3Gn_YYC-C-S C>T %; cds:A3Bf_ST-C-G Ti %; cds:2Gen2_G-C-Hits; cds:AIDd_WR-C-Y %; cds:A3F_T-C-G>C %; cds:4Gen3_CT-C-C C>T at MC1%; cds:AIDd_WR-C-Y G>C %; cds:A3Gi_SG-C-G MC2%; cds:Other MC3%; nc:2Gen1_-C-T C>A+G>T nc %; cds:3Gen2_G-C-T %; g:3Gen2_T-C-G C>T+G>A g %; cds:ADARc_SW-A-Y T>C cds %, and related metrics thereto.

The biological sample may have been obtained from the tissue type affected by the cancer. In some examples, the biological sample contains ovarian, breast, prostate, liver, colon, stomach, pancreatic, skin, thyroid, cervical, lymphoid, hematopoietic, bladder, lung, renal, rectal, uterine, and head or neck tissue or cells.

### BRIEF DESCRIPTION OF THE FIGURES

An example of the present invention will now be described with reference to the accompanying drawings, in which: —

FIG. 1 is a flow chart of an example of a method for generating a progression indicator for assessing the likelihood of cancer progression or recurrence in a subject.

FIG. 2 is a flow chart of an example of a process for training a computational model.

FIG. 3 is a schematic diagram of an example of a network architecture.

FIG. 4 is a schematic diagram of an example of a processing system.

FIG. 5 is a schematic diagram of an example of a client device.

FIG. 6 is a flow chart of a specific example of a method of generating a progression indicator for assessing the likelihood of cancer progression or recurrence in a subject.

FIG. 7 shows the results of applying a model to predict patient outcome in the mesothelioma (MESO) validation dataset. A) 11 patients were classified as either “High-PFS” (i.e. patients whose cancer did not progress before 12 months), or “Low-PFS” (i.e. patients whose cancer did progress before 12 months). All patients in the validation dataset were correctly classified as “High_PFS” or “Low_PFS” The overall accuracy of prediction was 100% (Accuracy: 100%, Sensitivity: 1, Specificity: 1). 100% of validation patients were correctly classified as “High_PFS” (3/3) and 100% were correctly classified as “Low_PFS” (8/8). B). Kaplan-Meier curves, including log-rank statistical tests for comparison of PFS distributions.

FIG. 8 shows the results of applying a model to predict patient outcome in the Adrenocortical Carcinoma (ADCC) validation dataset. A) 13 patients were classified as either “High-PFS” (i.e. patients whose cancer did not progress before 24 months), or “Low-PFS” (i.e. patients whose cancer did progress before 24 months). The overall accuracy of predictions was 100% (Accuracy: 100%, Sensitivity: 1.00, Specificity: 1.00): 100% of validation patients were correctly classified as “High_PFS” (7/7) and 100% were correctly classified as “Low_PFS” (6/6). B). Kaplan-Meier curves, including log-rank statistical tests for comparison of PFS distributions.

FIG. 9 shows the results of applying a model to predict patient outcome in the Lower Grade Glioma (BLGG) validation dataset. A) 44 patients were classified as either “High-PFS” (i.e. patients whose cancer did not progress before 24 months) in red, or “Low-PFS” (i.e. patients whose cancer did progress before 24 months). The overall accuracy of predictions was 84% (Accuracy: 84.09%, Sensitivity: 0.8846, Specificity: 0.7778): 88% of validation patients were correctly classified as “High_PFS” (23/26) and 77% were correctly classified as “Low_PFS” (14/18). B). Kaplan-Meier curves, including log-rank statistical tests for comparison of PFS distributions.

FIG. 10 shows the results of applying a model to predict patient outcome in the Sarcoma (SARC) validation dataset. A) 31 patients were classified as “High-PFS” (i.e. patients whose cancer did not progress before 18 months), or “Low-PFS” (i.e. patients whose cancer did progress before 18 months). The overall accuracy of predictions was 81% (Accuracy: 80.65%, Sensitivity: 0.9500, Specificity: 0.5455): 95% of validation patients were correctly classified as “High_PFS” (19/20) and 54.55% were correctly classified as “Low_PFS” (6/11). B). Kaplan-Meier curves, including log-rank statistical tests for comparison of PFS distributions.

FIG. 11 shows the results of applying a model to predict patient outcome in the Lung Squamous Cell Carcinoma (LUSC) validation dataset. 43 patients were classified as either “High-PFS” (i.e. patients whose cancer did not progress before 36 months), or “Low-PFS” (i.e. patients whose cancer did progress before 36 months). The overall accuracy of predictions was 67% (Accuracy: 67.44%, Sensitivity: 0.7586, Specificity: 0.500): 75.86% of validation patients were correctly classified as “High_PFS” (22/29) and 50% were correctly classified as “Low_PFS” (7/14). B). Kaplan-Meier curves, including log-rank statistical tests for comparison of PFS distributions.

FIG. 12 shows the results of applying a model to predict patient outcome in the Melanoma (SKCM) validation dataset. 56 patients were classified as “High-PFS” (i.e. patients whose cancer did not progress before 30 months), or “Low-PFS” (i.e. patients whose cancer did progress before 30 months). The overall accuracy of predictions was 73% (Accuracy: 73.21%, Sensitivity: 0.8485, Specificity: 0.5652): 84.85% of validation patients were correctly classified as “High_PFS” (28/33) and 56.52% were correctly classified as “Low_PFS” (13/23). B) Kaplan-Meier curves, including log-rank statistical tests for comparison of PFS distributions.

### DETAILED DESCRIPTION OF THE INVENTION

### I. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “a glycospecies biomarker” means one glycospecies biomarker or more than one glycospecies biomarker.

As used herein, “and/or” refers to and encompasses any and all possible combinations of one or more of the associated listed items, as well as the lack of combinations when interpreted in the alternative (or).

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The term “biological sample” as used herein refers to a sample that may be extracted, untreated, treated, diluted or concentrated from a subject or patient. Suitably, the biological sample is selected from any part of a patient's body, including, but not limited to hair, skin, nails, tissues or bodily fluids such as saliva and blood. For purposes of the present disclosure, a biological sample typically comprises cancer or tumour cells or tissue.

As used herein, the term “codon context” with reference to an SNV refers to the nucleotide position within a codon at which the SNV occurs. For the purposes of the present disclosure, the nucleotide positions within an affected codon (MC; i.e., a codon containing the SNV) are annotated MC-1, MC-2 and MC-3, and refer to the first, second and third nucleotide positions, respectively, when the sequence of the codon is read 5′ to 3′. Accordingly, the phrase “determining the codon context of an SNV” or similar phrase means determining at which nucleotide position within the affected codon the SNV occurs, i.e., MC-1, MC-2 or MC-3.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “control subject” or “reference subject”, as used in the context of the present disclosure refers to a subject whose cancer progression or recurrence is known, e.g. has or had a cancer that did not progress or recur, or has or had a cancer that did progress or recur. It is understood that control or reference subjects can be used to obtain data for use as a standard for multiple studies, i.e., it can be used over and over again for multiple different subjects. In other words, for example, when comparing a subject sample to a control or reference sample, the data from the control or reference sample could have been obtained in a different set of experiments, for example, it could be an average obtained from a number of subjects and not actually obtained at the time the data for the test subject was obtained.

The term “correlating” generally refers to determining a relationship between one type of data with another or with a state. In various embodiments, correlating a profile with the likelihood that a subject has a cancer that will progress or recur comprises assessing metrics as described herein in a subject and comparing the levels of these metrics to metrics in persons (such as represented by a reference profile) known have or have had a cancer that did or did not progress or recur.

By “gene” is meant a unit of inheritance that occupies a specific locus on a genome and comprises transcriptional and/or translational regulatory sequences and/or a coding region and/or non-translated sequences (i.e., introns, 5′ and 3′ untranslated sequences).

As used herein, the term “likelihood” or grammatical variations is used as a measure of whether the subject has a cancer that will progress or recur, such as within a particular timeframe and/or by a particular degree. An increased likelihood for example may be relative or absolute and may be expressed qualitatively or quantitatively. For instance, an increased likelihood that a cancer will progress or recur may be expressed as determining whether the subject has a profile of metrics that is essentially the same as or is different to a reference profile, and placing the test subject in an “increased likelihood” category or “decreased likelihood” category.

In some embodiments, the methods comprise comparing a score based on the number of metrics in a metric set that are outside a predetermined range interval or above or below a cut-off to a “threshold score”. The threshold score is one that provides an acceptable ability to identify a subject as having a cancer that is likely to progress or recur, and a subject as having a cancer that is unlikely to progress or recur, and can be determined by those skilled in the art using any acceptable means.

In some examples when determining likelihood, receiver operating characteristic (ROC) curves are calculated by plotting the value of a variable versus its relative frequency in two populations in which a first population has a first phenotype or risk and a second population has a second phenotype or risk. A distribution of the value of particular metrics, or in the number of metrics that are outside a predetermined range interval or are above or below a cutoff, in subjects whose cancer will progress or recur and in subjects cancer will not progress or recur, may overlap. Under such conditions, a test does not absolutely distinguish between the two groups with 100% accuracy. A threshold may be selected, above which the test is considered to be “positive” and below which the test is considered to be “negative.” The area under the ROC curve (AUC) provides the C-statistic, which is a measure of the probability that the perceived measurement will allow correct identification of a condition (see, for example, Hanley et al, Radiology 143: 29-36 (1982)). The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest. ROC curves are useful for plotting the performance of a particular feature in distinguishing or discriminating between two populations. Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features (e.g., one or more other epigenetic markers), in which the combination derives a single output value, can be plotted in a ROC curve. These combinations of features may comprise a test. The ROC curve is the plot of the sensitivity of a test against the specificity of the test, where sensitivity is traditionally presented on the vertical axis and specificity is traditionally presented on the horizontal axis. Thus, “AUC ROC values” are equal to the probability that a classifier will rank a randomly chosen positive instance higher than a randomly chosen negative one. An AUC ROC value may be thought of as equivalent to the Mann-Whitney U test, which tests for the median difference between scores obtained in the two groups considered if the groups are of continuous data, or to the Wilcoxon test of ranks.

As used herein, “level” with reference to a SNV or metric refers to the number, percentage, amount or ratio of SNV or metric.

As used herein, a “metric” refers to a number, percentage, ratio and/or type of a single nucleotide variant (SNV). The metrics of the present disclosure are associated with, reflective of or indicative of the number, percentage or ratio of particular SNVs, such as SNVs in the coding region of a nucleic acid molecule; SNVs in the non-coding region of a nucleic acid molecule; SNVs in both the coding and non-coding region of a nucleic acid molecule; SNVs where the coding context of the SNV has been assessed; SNVs that have been determined to be transitions or transversions; SNVs that have been determined to be synonymous or non-synonymous; SNVs resulting from or associated with strand bias; SNVs in which an adenine and thymine, and/or a guanine and cytidine have been targeted; SNVs present in specific motifs (e.g. deaminase or 3-mer motifs); and SNVs whether present in motifs or not (i.e. motif-independent metric group).

As used herein, an “SNV type” refers to the specific nucleotide substitution that comprises the SNV, and is selected from among C to T, C to A, C to G, G to T, G to A, G to C, A to T, A to C, A to G, T to A, T to C and T to G SNVs. Thus, for example, a C to T SNV refers to an SNV in which the targeted nucleotide C is replaced with the substituting nucleotide T.

The “nucleic acid” as used herein designates DNA, cDNA, mRNA, RNA, rRNA or cRNA. The term typically refers to polynucleotides greater than 30 nucleotide residues in length.

As used herein, a “predetermined range interval” refers to a range of values, with an upper and lower limit, for a metric that represents a “normal” range of values for the metric. The predetermined range interval can be determined by assessing a metric in two or more control subjects. A range interval is then calculated to set the upper and lower limits of what would be considered normal values for that metric in that control subject. In a particular example, the range interval is calculated by measuring the average plus or minus n standard deviations, whereby the lower limit of the range interval is the average minus n standard deviations and the upper limit of the range interval is the average plus n standard deviations. In still further examples, the upper and lower limits of the predetermined range interval are established using receiver operating characteristic (ROC) curves. The subjects used to determine the predetermined range interval can be of any age, sex or background, or may be of a particular age, sex, ethnic background or other subpopulation. Thus, in some embodiments, two or more range intervals can be calculated for the same metric, whereby each range interval is specific for a particular subpopulation, e.g. a particular sex, age group, ethnic background and/or other subpopulation. The predetermined range interval can be determined using any technique known to those skilled in the art, including manual methods of calculation, an algorithm, a neural network, a support vector machine, deep learning, logistic regression with linear models, machine learning, artificial intelligence and/or a Bayesian network.

As used herein, a “cut-off” with reference to a metric refers to an upper or lower limit of a value for a metric, above or below which represents a “normal” range of values for the metric for that phenotype (e.g. for a cancer that is likely to progress or recur, and for a cancer that is unlikely to progress or recur). The cut-off can be determined by assessing a metric in two or more control subjects. A cut-off is then calculated to set an upper or lower limits of what would be considered normal values for that metric. In a particular example, the cut-off is calculated by measuring the average plus or minus n standard deviations, whereby a lower limit cut-off is the average minus n standard deviations and an upper limit cut-off is the average plus n standard deviations. In still further examples, the cut-offs are established using receiver operating characteristic (ROC) curves. The subjects used to determine the cut-off can be of any age, sex or background, or may be of a particular age, sex, ethnic background or other subpopulation. Thus, in some embodiments, two or more cut-offs can be calculated for the same metric, whereby each cut-off is specific for a particular subpopulation, e.g. a particular sex, age group, ethnic background and/or other subpopulation. The cut-off can be determined using any technique known to those skilled in the art, including manual methods of calculation, an algorithm, a neural network, a support vector machine, deep learning, logistic regression with linear models, machine learning, artificial intelligence and/or a Bayesian network.

As used herein, the terms “recur,” “recurrence” and the like refer to the re-growth of tumour or cancerous cells in a subject after a primary treatment for the cancer or tumour has been successfully administered (i.e. after the primary treatment resulted in partial or complete regression of the cancer or tumour, for a period of time). The tumour may recur in the original site or in another part of the body. In one embodiment, a tumour that recurs is of the same type as the original tumour for which the subject was treated. For example, if a subject had an ovarian cancer tumour, was treated for and subsequently developed another ovarian cancer tumour, the tumour has recurred. In addition, a cancer can recur in or metastasize to a different organ or tissue than the organ or tissue where it originally occurred.

As used herein, the terms “progress,” “progression” and the like refer to any measure of cancer growth, development, and/or maturation, including metastasis. Cancer progression includes, for example, an increase in cancer cell number, cancer cell size, tumour size, and number of tumours, as well as morphological and other cellular and molecular changes and other characteristics, and can occur before, during or after primary or subsequent treatment. Progression can be assessed and expressed in any suitable manner, and may be in absolute terms (e.g. has or will the cancer progress or recur), or in terms of a time frame (e.g. has or will the cancer progress or recur within a given timeframe). In one example, progression is expressed as progression free survival (PFS) time, e.g. length of time (in some cases, during and after the treatment of the cancer) that the cancer does not progress or the patient does not die. In such examples, a determination that a subject has a cancer that is likely to progress may be a determination that a subject has a relatively low (e.g. a set number of months or years) PFS time, while a determination that a subject has a cancer that is unlikely to progress may be a determination that a subject has a relatively high PFS time.

The term “sensitivity”, as used herein, refers to the probability that a predictive method or kit of the present disclosure gives a positive result when the biological sample is positive, e.g., having the predicted diagnosis. Sensitivity is calculated as the number of true positive results divided by the sum of the true positives and false negatives. Sensitivity essentially is a measure of how well the present disclosure correctly identifies those who have the predicted diagnosis from those who do not have the predicted diagnosis. The statistical methods and models can be selected such that the sensitivity is at least about 50%, and can be, e.g., at least about 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

The term “specificity” as used herein, refers to the probability that a predictive method or kit of the present disclosure can distinguish between a positive and negative result, e.g. between two diagnoses. Specificity is calculated as the number of true negative results divided by the sum of the true negatives and false positives. The statistical methods and models can be selected such that the specificity is at least about 50%, and can be, e.g., at least about 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

As used herein, “single nucleotide variant”, “SNV” or “variant” refers to a variation occurring in the sequence of a nucleic acid molecule (e.g. a subject nucleic acid molecule) compared to another nucleic acid molecule (e.g. a reference nucleic acid molecule or sequence), wherein the variation is a difference in the identity of a single nucleotide (e.g. A, T, C or G). Reference to, for example, an “A variant” or “A SNV” means a variant or SNV in which an A is the mutated or targeted nucleotide. Reference to, for example, an “A>G variant” or “A>G SNV” means a variant or SNV in which an A is replaced with a G.

The terms “subject”, “individual” or “patient”, used interchangeably herein, refer to any animal subject, particularly a mammalian subject. By way of an illustrative example, suitable subjects are humans.

The terms “treat” and “treating” as used herein, unless otherwise indicated, refer to both therapeutic treatment and prophylactic or preventative measures, wherein the object is to inhibit, either partially or completely, ameliorate or slow down (lessen) one or more symptom associated with a disorder or condition, e.g. a cancer, for example, to reduce the size or number of tumours or cancer cells, or the rate of growth or spread of the cancer or tumour. The term “treatment” as used herein, unless otherwise indicated, refers to the act of treating.

As used herein, the term “treatment regimen” refers to a therapeutic regimen (i.e., after the diagnosis of a cancer, or of cancer progression or recurrence). The term “treatment regimen” encompasses natural substances and pharmaceutical agents as well as any other treatment regimen.

TABLE A
Nucleotide Symbols

| | |
|---|---|
| A | Adenine |
| C | Cytosine |
| G | Guanine |
| T | Thymine |
| U | Uracil |
| R | Purine - A or G |
| Y | Pyrimidine - C or T |
| S | G or C |
| W | A or T |
| K | G or T |
| M | A or C |
| B | C or G or T |
| D | A or G or T |
| H | A or C or T |
| V | A or C or G |
| N | any base |

Those skilled in the art will appreciate that the aspects and embodiments described herein are susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

### 2. Metrics

As described herein, SNVs identified in a nucleic acid molecule can be used to determine a plurality of metrics. For the purpose of the present disclosure, specific metrics have consequently been determined to be CPAS, and these CPAS can be used to develop a profile that can be used to distinguish subjects for whom their cancer is likely to progress or recur from subjects for whom their cancer is unlikely to progress or recur.

As will be appreciated from the description below, the metrics are determined based on the number or percentage of SNVs in any one or more regions of the nucleic acid molecules, and can include an assessment of the targeted nucleotide (i.e. whether the targeted nucleotide is an A, T, C or G), the type of SNV (e.g. whether the targeted nucleotide is now an A, T, G or C), whether the SNV is a transition or transversion SNV and/or whether the SNV is synonymous or non-synonymous, the motif in which the targeted nucleotide resides, the codon context of the SNV, and/or the strand on which the SNV occurs. Any single SNV can therefore be used to generate one or more metrics, and multiple SNVs can be used to generate two more metrics, and typically at least 10, 20, 30, 40, 50, 60, 70, 80, 90, 100 or more metrics. A profile can be built based upon this plurality of metrics, whereupon subjects that have a cancer that is likely to progress or recur typically have a different profile to subjects that have a cancer (e.g. a cancer of the same type) that is unlikely to progress or recur.

As will be apparent from the disclosure herein, metrics can be associated with or indicative of deaminase activity, i.e. the metrics reflect a number, percentage, ratio and/or type of SNV that may be indicative of the activity of one or more endogenous deaminases, e.g. ADAR, AID or an APOBEC deaminase (e.g. APOBEC1, APOBEC3B, APOBEC3F or APOBEC3G).

Any one or more of the metrics can be assessed for the methods of the present disclosure. Typically, multiple metrics are assessed, such as at least 4, 5, 6, 7, 8, 9, 10, 11, 12, 13, 14, 15, 20, 40, 60, 80, 100 or more.

2.1 Motifs

In instances where the metrics are determined using SNVs identified within a particular motif (i.e. metrics in the motif metric group), motifs may be analysed in pairs: the forward motif and the equivalent reverse complement motif. For example, a forward motif ACG represents a motif in which the underlined C is targeted (or modified), and the reverse motif is CGT, where the underlined G is targeted (or modified). As would be understood, identifying a reverse compliment motif is equivalent to identifying the forward motif on the reverse compliment DNA strand. For purposes herein, the targeted/mutated nucleotide, which is underlined in the previous passage, can also be identified by the presence of hyphens either side, i.e. “ACG” is the equivalent to “A-C-G” (where the targeted C is either underlined or framed by hyphens), and “CGT” is the equivalent to “CG-T-” (where the targeted T is either underlined or framed by hyphens).

Motifs include those that are known or suggested deaminase motifs. Thus, the metrics may be associated with SNVs in one or more deaminase motifs. Such metrics can therefore also be referred to as genetic indicators of deaminase activity.

Table B sets forth exemplary deaminase motifs utilised for determination of the metrics of the present disclosure. The primary motif for AID is WR-C-/-G-YW and secondary motifs include, for example, AIDb, c, d, e, f, g and h. The primary motif for ADAR is W-A-/-T-W (where the mutated/targeted base is A or T) and secondary motifs include ADARb, c, d, e, f, g, h, l, j, k, n and p. The primary motif for APOBEC3G (A3G) is C-C-/-G-G (where the mutated/targeted base is C or G), and secondary motifs include A3Gb, c, d, e, f, g, h, i, n, and o. The primary motif for APOBEC3B (A3B) is T-C-W/W-G-A (where the mutated/targeted base is C or G), and secondary motifs include, for example, A3Bb, c, d, e, f, g, h, and j. The motif for APOBEC3F (A3F) is T-C-/-G-A (where the mutated/targeted base is C or G) and the motif for APOBEC1 (A1) is —C-A/T-G- (where the mutated/targeted base is C or G).

Thus, reference to a “primary motif” herein is reference to any one of WR-C-/-G-YW, W-A-/-T-W, C-C-/-G-G, and T-C-W/W-G-A (i.e. the first four motifs in Table B below). Any SNV that is not at a primary motif, is considered as an “other” SNV (i.e. “other” SNVs include any SNV that is not at one of the four primary motifs, including SNVs that are not at any motif and SNVs that are at secondary or other motifs).

TABLE B
Exemplary deaminase motifs

| Motif Name | Forward motif | | Reverse motif |
|---|---|---|---|
| AID | WR-C- | / | -G-YW |
| ADAR | W-A- | / | -T-W |
| A3G | C-C- | / | -G-G |
| A3B | T-C-W | / | W-G-A |
| AIDb | WR-C-G | / | C-G-YW |
| AIDC | WR-C-GS | / | SC-G-YW |
| AIDd | WR-C-Y | / | R-G-YW |
| AIDe | WR-C-GW | / | WC-G-YW |
| AIDh | WR-C-T | / | A-G-YW |
| ADARb | W-A-Y | / | R-T-W |
| ADARc | SW-A-Y | / | R-T-WS |
| ADARf | SW-A- | / | -T-WS |
| ADARh | W-A-S | / | S-T-W |
| ADARk | CW-A- | / | -T-WG |
| ADARn | -A-WA | / | TW-T- |
| ADARp | -A-WT | / | AW-T- |
| A3Gb | -C-G | / | C-G- |
| A3Gc | C-C-GW | / | WC-G-G |
| A3Ge | SC-C-GS | / | SC-G-GS |
| A3Gi | SG-C-G | / | C-G-CS |
| A3Gn | YYC-C-S | / | S-G-GRR |
| A3Go | TC-C-G | / | C-G-GA |
| A3Bf | ST-C-G | / | C-G-AS |
| A3BJ | RT-C-G | / | C-G-AY |
| A3F | T-C- | / | -G-A |
| A1 | -C-A | / | T-G- |

In further examples, the motifs are not necessarily deaminase motifs. Included among such motifs are general 2-mer motifs in which a SNV is detected in one of the positions in the 2-mer: M1 or M2. Also included among such motifs are general 3-mer motifs in which a SNV is detected in one of the positions in the 3-mer: M1, M2 or M3. Also included are general 4-mer motifs, in which a SNV is detected in one of the positions in the 4-mer: M1, M2, M3 or M4. Motifs not known to be specifically associated with deaminase enzymes are labelled herein as “Gen” motifs; and “ADAR_Gen” is used to identify motifs where A or T is the targeted (or mutated) nucleotide. The first, second or third nucleotide (i.e. M1, M2 or M3) is typically the targeted nucleotide. For purposes herein, “2Gen1” indicates a two nucleotide motif where the first position is the targeted nucleotide, e.g. “2Gen1_-G-T” is a 2-mer motif where the G in the first position is the targeted nucleotide (or C in the reverse motif). “3Gen1” is a 3-mer motif where the first position is the targeted nucleotide, e.g. “3Gen1_-C-TA” is a three nucleotide motif where the C at the first position is the targeted nucleotide (or G in the reverse motif). “3Gen2” is a 3-mer motif where the second position is the targeted nucleotide, e.g. “ADAR_3 Gen2_G-A-T” is a 3-mer motif where the A at the second position is the targeted nucleotide (or the T in the reverse motif). “3Gen3” is a 3-mer motif where the third position is the targeted nucleotide, e.g. “3Gen3_GA-C” is a 3-mer motif where the C at the third position is the targeted nucleotide (or the G in the reverse motif). “4Gen3” is a 4-mer motif where the third position is the targeted nucleotide, e.g. “ADAR_4 Gen3_AT-A-T” is a 4-mer motif where the A at the third position is the targeted nucleotide (or the T in the reverse motif).

Non-limiting examples of general motifs include those set forth in Table C below.

TABLE C
Exemplary general motifs

| Motif Name | Forward motif | | Reverse motif |
|---|---|---|---|
| 2Gen1_-C-C | -C-C | / | G-G- |
| 2Gen1_-C-T | -C-T | / | A-G- |
| 2Gen2_G-C- | G-C- | / | -G-C |
| 2Gen2_A-C- | A-C- | / | -G-T |
| ADAR_2Gen1_-T-T | -T-T | / | A-A- |
| ADAR_2Gen2_A-T- | A-T- | / | -A-T |
| ADAR_2Gen2_T-T- | T-T- | / | -A-A |
| ADAR_2Gen2_G-T- | G-T- | / | -A-C |
| 3Gen1_-C-CA | -C-CA | / | TG-G- |
| 3Gen1_-C-CT | -C-CT | / | AG-G- |
| 3Gen1_-C-GT | -C-GT | / | AC-G- |
| 3Gen1_-C-TC | -C-TC | / | GA-G- |
| 3Gen1_-C-CC | -C-CC | / | GG-G- |
| 3Gen1_-C-GC | -C-GC | / | GC-G- |
| 3Gen1_-C-AG | -C-AG | / | CT-G- |
| 3Gen1_-C-TG | -C-TG | / | CA-G- |
| 3Gen1_-C-CG | -C-CG | / | CG-G- |
| 3Gen2_T-C-T | T-C-T | / | A-G-A |
| 3Gen2_C-C-T | C-C-T | / | A-G-G |
| 3Gen2_G-C-T | G-C-T | / | A-G-C |
| 3Gen2_A-C-C | A-C-C | / | G-G-T |
| 3Gen2_T-C-C | T-C-C | / | G-G-A |
| 3Gen2_C-C-C | C-C-C | / | G-G-G |
| 3Gen2_A-C-G | A-C-G | / | C-G-T |
| 3Gen2_T-C-G | T-C-G | / | C-G-A |
| 3Gen3_AT-C- | AT-C- | / | -G-AT |
| 3Gen3_AG-C- | AG-C- | / | -G-CT |
| 3Gen3_TT-C- | TT-C- | / | -G-AA |
| 3Gen3_TG-C- | TG-C- | / | -G-CA |
| 3Gen3_CA-C- | CA-C- | / | -G-TG |
| 3Gen3_CT-C- | CT-C- | / | -G-AG |
| 3Gen3_GA-C- | GA-C- | / | -G-TC |
| 3Gen3_GG-C- | GG-C- | / | -G-CC |
| ADAR_3Gen1_-A-AT | -A-AT | / | AT-T- |
| ADAR_3Gen1_-A-CA | -A-CA | / | TG-T- |
| ADAR_3Gen1_-A-CC | -A-CC | / | GG-T- |
| ADAR_3Gen2_C-A-C | C-A-C | / | G-T-G |
| ADAR_3Gen2_G-A-C | G-A-C | / | G-T-C |
| ADAR_3Gen3_AC-A- | AC-A- | / | -T-GT |
| ADAR_3Gen3_CA-A- | CA-A- | / | -T-TG |
| ADAR_3Gen3_CT-A- | CT-A- | / | -T-AG |
| 4Gen3_AC-C-T | AC-C-T | / | A-G-GT |
| 4Gen3_AG-C-T | AG-C-T | / | A-G-CT |
| 4Gen3_CA-C-C | CA-C-C | / | G-G-TG |
| 4Gen3_CT-C-C | CT-C-C | / | G-G-AG |
| 4Gen3_GC-C-A | GC-C-A | / | T-G-GC |
| 4Gen3_GC-C-C | GC-C-C | / | G-G-GC |
| 4Gen3_GG-C-G | GG-C-G | / | C-G-CC |
| 4Gen3_TA-C-C | TA-C-C | / | G-G-TA |
| 4Gen3_TG-C-T | TG-C-T | / | A-G-CA |
| 4Gen3_TT-C-C | TT-C-C | / | G-G-AA |
| 4Gen3_TT-C-T | TT-C-T | / | A-G-AA |
| ADAR_4Gen3_AG-A-G | AG-A-G | / | C-T-CT |

The motif metrics may reflect (and thus be generated by assessing) the number or percentage of total SNVs in the nucleic acid molecules that are at a particular motif. In further embodiments, motif metrics can be generated by detecting, and can therefore indicate, the particular type of SNV at the targeted nucleotide, e.g. whether there is an A, C or T substituting a targeted G. Further, the metrics can indicate whether the targeted nucleotide is at any position within the codon (i.e. at MC-1, MC-2 or MC-3, as described below). Thus, in some examples, motif metrics can represent a number, percentage or ratio of any SNV at a targeted position in a motif (e.g. a deaminase motif), wherein the targeted nucleotide is at any position within the codon. The percentage of SNVs at the motif is therefore calculated by dividing the total number of SNVs at the motif (regardless of the type of the mutation or codon context of the mutation) by the total number of SNVs in nucleic acid molecule. In other examples, however, only SNVs that are particular types of SNV, such as transition SNVs (i.e. C>T, G>A, T>C and A>G), at a motif are considered in the assessment and metric reflects the percentage, number or ratio of such SNVs. In still further examples, only SNV that result in a synonymous mutation, or that result in a non-synonymous mutation, are considered. In still further embodiments, both the codon context and the type of SNV is assessed, as described below.

2.2 Codon Context

Mutagens, including deaminases, can target nucleotides in a codon context manner (as described in, for example, WO 2014/066955 and Lindley et al. (2016) Cancer Med. 2016 September; 5(9): 2629-2640). Specifically, mutagenesis can occur at a targeted nucleotide, wherein the targeted nucleotide is present at a particular position within a codon. For the purposes of the present disclosure, the nucleotide positions within an affected codon (MC; i.e., a codon containing the SNV) are annotated MC-1, MC-2 and MC-3, and refer to the first, second and third nucleotide positions, respectively, of the codon when the sequence of the codon is read 5′ to 3′.

Metrics of the present disclosure can be based, at least in part, on a determination of the codon context of an SNV, i.e. whether the SNV is at the first, second or third position in the affected codon, i.e. the MC-1, MC-2 or MC-3 site. As noted above, many deaminases have a preference for targeting nucleotides at a particular position within the affected codon. As such, the number and/or percentage of SNVs that occur at a MC-1, MC-2 or MC-3 site can be a genetic indicator of deaminase activity. As would be appreciated, codon-context metrics are only assessed in the coding region of the nucleic acid molecule.

Metrics based on an assessment of the codon context of an SNV can be motif-independent (i.e. an assessment of the number and/or percentage of SNVs at a particular codon regardless of whether or not the targeted nucleotide is within a particular motif). Thus, these metrics include the number and/or percentage of total SNVs that occur at a MC-1 site; the number and/or percentage of total SNVs that occur at a MC-2 site; and or the number and/or percentage of total SNVs that occur at a MC-3 site.

In other embodiments, a simultaneous assessment of whether the SNV is at a motif, such as a deaminase motif, 3-mer motif or five-mer motif (as described above) is also made. Thus, the metrics include codon-context, motif-dependent metrics that are based on the number and/or percentage of SNVs within in a particular motif and at a MC-1 site, MC-2 site and/or MC-3 site. Where the motifs are deaminase motifs, the metrics can be considered as genetic indicators of deaminase activity, and include the number and/or percentage of SNVs that are attributable to a particular motif at a MC-1 site, MC-2 site and/or MC-3 site, such as the number and/or percentage of SNVs that are attributable to AID (i.e. that are at an AID motif) and that occur at a MC-1 site, MC-2 site and/or MC-3 site; the number and/or percentage of SNVs that are attributable to ADAR (i.e. that are at an ADAR motif) and that occur at a MC-1 site, a MC-2 site and/or a MC-3 site; the number and/or percentage of SNVs that are attributable to an APOBEC deaminase (i.e. that are at an APOBEC motif, such as a APOBEC1, APOBEC3A, APOBEC3B, APOBEC3C, APOBEC3D, APOBEC3F, APOBEC3G or APOBEC3H motif) and that occur at a MC-1 site, MC-2 site and/or a MC-3 site.

The codon-context metrics also include those that take into account not only the codon context, but also the nucleotide that is targeted. Thus, the metrics include the number or percentage of SNVs resulting from an adenine which are at the MC1 position, MC2 position and/or MC3 position. For example, the number of SNVs resulting from an adenine may be determined, and the percentage of these that are at a MC-1 site, MC-2 site and/or MC-3 site is then determined to generate the metric. Similarly, the number or percentage of SNVs resulting from a thymine that occurred at the MC1 position, the MC2 position and/or the MC3 position; the number or percentage of SNVs resulting from a cytosine that occurred at the MC1 position, the MC2 position, and/or the MC3 position; the number or percentage of SNVs resulting from a guanine that occurred at the MC1 position, the MC2 position, and/or the MC3 position can be assessed to generate the metrics.

In further embodiments, both the type of SNV (e.g. C>A, C>T, C>G, G>C, G>T, G>A, A>T, A>G, A>C, T>A, T>C or T>G) and the codon context of the SNV is assessed, so as to determine the number or percentage of a particular type of SNV at a MC-1, MC-2 or MC-3 site. Again, in some embodiments, this is performed without a simultaneous assessment of whether the SNV is at a motif associated with a particular deaminase. Thus, metrics may include, for example, the number or percentage of C>T SNVs at the MC1 site (typically indicative of AID, APOBEC3B or APOBEC3G activity); the number or percentage of C>T SNVs at the MC2 site (typically indicative of AID, APOBEC3B or APOBEC3G activity); the number or percentage of C>T SNVs at the MC3 site (typically indicative of AID, APOBEC3B or APOBEC3G activity); the number or percentage of G>A SNVs at the MC1 site (typically indicative of AID, APOBEC3B or APOBEC3G activity); the number or percentage of G>A SNVs at the MC2 site (typically indicative of AID, APOBEC3B or APOBEC3G activity); the number or percentage of G>A SNVs at the MC3 site (typically indicative of AID, APOBEC3B or APOBEC3G activity); the number or percentage of T>C SNVs at the MC1 site (typically indicative of ADAR activity); the number or percentage of T>C SNVs at the MC2 site (typically indicative of ADAR activity); the number or percentage of T>C SNVs at the MC3 site (typically indicative of ADAR activity); the number or percentage of A>G SNVs at the MC1 site (typically indicative of ADAR activity); the number or percentage of A>G SNVs at the MC2 site (typically indicative of ADAR activity); and the number or percentage of A>G SNVs at the MC3 site (typically indicative of ADAR activity).

In other embodiments, an assessment of whether the SNV is at a motif (e.g. a deaminase or 3-mer), what type of SNV is identified, and also the codon context of the SNV is made to generate the metric.

2.3 Transitions/Transversions

Transitions (Ti) are defined as any variant of a purine to a purine, or a pyrimidine to a pyrimidine (i.e. C>T, G>A, T>C and A>G), and transversions (Tv) are defined as any variant of a pyrimidine to a purine or purine to a pyrimidine (i.e. C>A, C>G, G>T, G>C, T>G, A>T, T>C and T>A). Metrics determined from or associated with SNVs that are transitions or transversions can thus be determined, and include, for example, the number or percentage of SNVs that are transitions or transversions, or the ratio of transitions to transversions or transversions to transitions). In some embodiments, the motif, codon context and/or specific SNV type is also assessed.

2.4 Strand Specificity

Metrics can also include those based on SNVs identified on just one strand of DNA, i.e. the non-transcribed (or sense or coding) strand or the transcribed (or antisense or template) strand. The non-transcribed (or sense or coding) strand may also be referred to as the “C” strand when SNVs of/from C are assessed, or the “A” strand when SNVs of/from A are assessed, while the transcribed (or antisense or template) strand may also be referred to as the “G” strand when SNVs of/from G are assessed, or the “T” strand when SNVs of/from T are assessed. These strand specific metrics typically include an assessment of the number or percentage of SNVs from (or of) a particular targeted nucleotide (e.g. A, T, C or G) on a given strand. Given that particular deaminases can have a preference for targeting a particular nucleotide in a nucleic acid molecule, such metrics can be considered genetic indicators of deaminase activity. For example, adenines are often the target of ADAR, while cytosines are often the target of AID or APOBEC deaminases. Thus, metrics can represent the number or percentage of SNVs resulting from an adenine nucleotide (e.g. detecting the total number of SNVs of A>C, A>T and A>G and expressing this total as a percentage of the total number of SNVs detected); the number or percentage of SNVs resulting from a thymine nucleotide (e.g. detecting the total number of SNVs of T>C, T>A and T>G and expressing this total as a percentage of the total number of SNVs detected); the number or percentage of SNVs resulting from a cytosine nucleotide (e.g. detecting the total number of SNVs of C>A, C>T and C>G and expressing this total as a percentage of the total number of SNVs detected); and/or the number or percentage of SNVs resulting from a guanine nucleotide (e.g. detecting the total number of SNVs of G>C, G>T and G>A and expressing this total as a percentage of the total number of SNVs detected). These can also be an indication of strand bias, as they can show an imbalance in the total number of SNVs of A, T, G or C nucleotides. In a further example, the nucleotide to which the targeted nucleotide becomes is also assessed. For example, the metric may represent the number or percentage of all SNVs that target A that are A>C SNVs.

2.5 AT and GC SNVs

Metrics can also include an assessment of combined SNVs targeting adenine and thymine (AT) and/or combined SNVs targeting guanine and cytosine (GC). The number and/or percentage of SNVs at AT or GC can be assessed. In further instances, a ratio is calculated, such as a ratio of the number or percentage of SNVs that include an adenine or a thymine nucleotide to the number or percentage of SNVs that include a cytosine or a guanine nucleotide (AT:GC ratio) is determined. In further instances, the codon context of the AT or GC SNVs can be taken into consideration to generate the metrics.

2.6 Coding Region and Genomic Metrics

Metrics can be determined using SNVs identified in just the coding region (also referred to as the coding sequence or cds) of a nucleic acid molecule. Other exemplary metrics include those that are determined across all regions of the genomic nucleic acid sequence are assessed, i.e. regardless of whether the sequence is of a non-coding or coding region. As would be appreciated, these metrics can thus be determined and/or used when the sequence of only a part of the nucleic acid is assessed (e.g. by whole exome sequencing), or whether the sequence of the entire nucleic acid is assessed (e.g. by whole genome sequencing).

### 3. Exemplary Metrics that are CPAS

As determined herein, a number of metrics are CPAS and can be used in the methods described herein to generate a profile or model that is predictive of whether or not a cancer in a subject will progress or recur. Table D sets forth exemplary CPAS for use in accordance with the methods and systems of the present disclosure. The table provides the metric name, the region on which the metric determination is based, the motif associated with the metric (where applicable), and the description of the metric and the calculation performed to generate the metric.

The CPAS therefore include those metrics that are specific for the cds (i.e. calculated on the basis of SNVs in the cds, e.g. “cds:CDS Variants” which is the total number of SNVs in the cds); those that are calculated on the basis of SNVs in the non-coding region (“nc” in Table D); and those that are calculated on the basis of SNVs genome-wide (“g” in Table D), e.g. “variants in VCF” which is the total number of SNVs in the genome. Where the definition in Table D refers to “motif”, it is the motif that is noted in the metric name and in the “motif” column of Table D, and “motif SNVs” means the SNVs at that particular motif. For example, “cds:ADAR_W-A-A>G at MC3%” is the percentage of A>G SNVs at the W-A-motif that are at MC3, i.e. of all of A>G SNVs at the W-A-motif, the percentage that are at MC3. Reference to “motif” in the definition column of any of the tables presented herein therefore means the motif referred to in the metric name. For example, the definition “% of motif variants that are at MC3” for the “cds:3Gen2_C-C-C MC3%” metric means the percentage of C-C-C or the reverse complement G-G-G variants (or variants at the C-C-C/G-G-G motif) that are at MC3. Reference to “cds” in the metric name indicates that it is the SNVs in the CDS that are assessed for this metric, as expected for a metric that involves an assessment of codon context. In another example, “cds:ADAR_W-A-non-syn %” is the percentage of SNVs at the W-A-/-T-W motif in the cds that correspond to (or are) non-synonymous changes. In a further example, cds:A3G_C-C-G>T % refers to the percentage of “G motif SNVs” (i.e. SNVs at “G” on the reverse strand at the -G-G motif) that are G>T mutations. Any SNV that is not at a primary motif, is considered as an “other” SNV (i.e. “other” SNVs include any SNV that is not at one of the four primary motifs, including SNVs that are not at any motif and SNVs that are at secondary or other motifs). Thus, for example, cds:Other MC3% is the percentage of “other” SNVs in the cds (i.e. SNVs not at a primary motif in the CDS) that are at MC3.

In Table D, #CDS=the number of SNVs in the CDS; #SNVs=the number of SNVs in the genomic region; #motif=the number of SNVs at the recited motif; #motif_Gstrand=the number of SNVs at the recited motif on the G strand; #other=the number of SNVs that are not at the primary deaminases motifs. N/A=not applicable.

TABLE D
Exemplary metrics that are CPAS

| Metric | Region | Motif | Description | Calculation |
|---|---|---|---|---|
| variants in VCF | g | N/A | Total number of SNVs | #SNVs |
| cds: CDS Variants | cds | N/A | Total number of SNVs within the | #CDS |
| | | | coding region of the genome | |
| cds: ADAR_W-A- A > G at | cds | W-A- | % of A > G motif variants that | #motif_A > G_MC3/#motif_A > G |
| MC3% | | | are at MC3 | |
| cds: ADAR_W-A- T > C at | cds | W-A- | % of T > C motif variants that | #motif_T > C_MC2/#motif_T > C |
| MC2% | | | are at MC2 | |
| cds: ADAR_W-A- non-syn % | cds | W-A- | % of motif variants that correspond | #motif_nonsyn/#motif |
| | | | to non-synonymous changes | |
| g: ADAR_W-A- A > G + | g | W-A- | % of motif variants that are A > | (#motif_ A > G + #motif_T > C)/#motif |
| T > C % | | | G or T > C | |
| cds: A3G_C-C- MC3% | cds | C-C- | % of motif variants that are at | #motif_MC3/#motif |
| | | | MC3 | |
| cds: A3G_C-C- C > T at MC1% | cds | C-C- | % of C > T motif variants that | #motif_C > T_MC1/#motif_C > T |
| | | | are at MC1 | |
| cds: A3G_C-C- G > T % | cds | C-C- | % of G motif variants that are | #motif_G > T/#motif_Gstrand |
| | | | G > T | |
| nc: A3G_C-C- C > T + | nc | C-C- | % (of all non-coding variants) of | #nc_motif_Ti/#nc_SNVs |
| G > A nc % | | | motif variants that are transitions | |
| cds: A3B_T-C-W Ti % | cds | T-C-W | % of all cds variants that are | #motif_Ti/#CDS |
| | | | transition motif variants | |
| cds: A3B_T-C-W MC1% | cds | T-C-W | % of motif variants that are at MC1 | #motif_MC1/#motif |
| cds: A3B_T-C-W G non- | cds | T-C-W | % of G motif variants that are non- | #motif_Gstrand_nonsyn/#motif_Gstrand |
| syn % | | | synonymous | |
| cds: A3B_T-C-W MC3 non- | cds | T-C-W | % of MC3 motif variants that are | #motif_MC3_nonsyn/#motif_MC3 |
| syn % | | | non-synonymous | |
| cds: A3B_T-C-W G > A | cds | T-C-W | % of motif variants that are G > A | #motif_G > A/#motif |
| motif % | | | | |
| cds: Primary Deaminase % | cds | Primary | % of all cds variants that are | (#ADAR + #AID + A3G + |
| | | | “primary deaminase” motif | #A3B)/#CDS |
| | | | variants | |
| cds: All G total | cds | N/A | Number of cds variants that are at G | #G |
| cds: All G % | cds | N/A | % of all variants that are G | #G/#CDS |
| cds: All C Ti/Tv % | cds | N/A | % of all C cds variants that are | #C_Ti/#C |
| | | | transitions | |
| cds: All A non-syn % | cds | N/A | % of all A genomic variants that | #A_nonsyn/#A |
| | | | are non-synonymous | |
| g: CG total | g | CG | Number of variants at C or G | #C + #G |
| g: C > A + G > T % | g | N/A | % of all genomic variants that are | (#C > A + #G > T)/#SNVs |
| | | | C > A or G > T | |
| g: C > G + G > C % | g | N/A | % of all genomic variants that are | (#C > G + #G > C)/#SNVs |
| | | | C > G or G > C | |
| cds: Other MC3% | cds | N/A | % of “Other” variants that | #other_MC3/#other |
| | | | are MC3 | |
| cds: Other MC3 C % | cds | N/A | % of “Other” MC3 variants | #other_Cstrand_MC3/#other_MC3 |
| | | | that are at C | |
| cds Other G MC3 Ti/Tv % | cds | N/A | % of “Other” G strand MC3 | #other_Gstrand_MC3_Ti/#other_Gstrand_MC3 |
| | | | variants that are transitions | |
| cds: AIDb_WR-C-G G non- | cds | WR-C-G | % of G motif variants that are | #motif_Gstrand_nonsyn/#motif_Gstrand |
| syn % | | | non-synonymous | |
| cds: AIDc_WR-C-GS % | cds | WR-C-GS | % of all cds variants that are | #motif/#CDS |
| | | | motif variants | |
| cds: AIDc_WR-C-GS MC3% | cds | WR-C-GS | % of motif variants that are at | #motif_MC3/#motif |
| | | | MC3 | |
| g: AIDc_WR-C-GS Hits | g | WR-C-GS | Number of motif variants | #motif |
| cds: AIDd_WR-C-Y % | cds | WR-C-Y | % of all cds variants that are | #motif/#CDS |
| | | | motif variants | |
| cds: AIDd_WR-C-Y G > | cds | WR-C-Y | % of G motif variants that are | #motif_G > C/#motif_Gstrand |
| C % | | | G > C | |
| cds: AIDd_WR-C-Y C > A | cds | WR-C-Y | % (of all cds variants) that are | #motif_C > A/#CDS |
| cds % | | | C > A motif variants | |
| cds: AIDe_WR-C-GW Hits | cds | WR-C-GW | Number of motif variants | #motif |
| cds: AIDh_WR-C-T G > A | cds | WR-C-T | % (of all cds variants) of G > A | #motif_G > A_MC2/#CDS |
| at MC2 cds % | | | variants that are at MC2 | |
| g: AIDh_WR-C-T C > A + | g | WR-C-T | % (of all genomic variants) of | (#motif_C > A + #motif_G > T)/#SNVs |
| G > T g % | | | motif variants that are C > A or | |
| | | | G > T | |
| cds: ADARb_W-A-Y MC2% | cds | W-A-Y | % of motif variants that are at | #motif_MC2/#motif |
| | | | MC2 | |
| nc: ADARb_W-A-Y % | nc | W-A-Y | % (of all non-coding variants) | #nc_motif/#nc_SNVs |
| | | | that are motif variants | |
| nc: ADARb_W-A-Y A > G + | nc | W-A-Y | % (of all non-coding variants) of | (#nc_motif_A > G + #nc_motif_T > |
| T > C nc % | | | motif variants that are A > G or | C)/#nc_SNVs |
| | | | T > C | |
| cds: ADARc_SW-A-Y MC2% | cds | SW-A-Y | % of motif variants that are at | #motif_MC2/#motif |
| | | | MC2 | |
| cds: ADARc_SW-A-Y T > C | cds | SW-A-Y | % (of all cds variants) that are | #motif_T > C/#CDS |
| cds % | | | T > C motif variants | |
| nc: ADARc_SW-A-Y A > G + | nc | SW-A-Y | % (of all non-coding variants) | (#nc_motif_A > G + #nc_motif_T > |
| T > C nc % | | | that are T > C motif variants | C)/#nc_SNVs |
| cds: ADARf_SW-A- MC2% | cds | SW-A- | % of motif variants that are at | #motif_MC2/#motif |
| | | | MC2 | |
| cds: ADARh_W-A-S T > C % | cds | W-A-S | % of T motif variants that are | #motif_T > C/#motif |
| | | | T > C | |
| g: ADARk_CW-A- A > G + | g | CW-A- | % (of all genomic variants) of | (#motif_A > G or #motif_T > C)/#motif |
| T > C g % | | | motif variants that are A > G | |
| | | | or T > C | |
| g: ADARn_-A-WA A > G + | g | -A-WA | % (of all genomic variants) of | (#motif_A > G or #motif_T > C)/#motif |
| T > C % | | | motif variants that are A > G | |
| | | | or T > C | |
| cds: ADARp_-A-WT T > A | cds | -A-WT | % of motif variants that are | #motif_T > A/#motif |
| motif % | | | T > A | |
| cds: ADARp_-A-WT A > G | cds | -A-WT | % (of all cds variants) of A > | #motif_A > G_MC2/#CDS |
| at MC2 cds % | | | G variants that are at MC2 | |
| cds: A3Gb_-C-G G > A | cds | -C-G | Number of G > A motif variants | #motif_G > A_MC2 |
| MC2 Hits | | | at MC2 positions | |
| cds: A3Gb_-C-G MC1% | cds | -C-G | % of motif variants that are at | #motif_MC1/#motif |
| | | | MC1 | |
| cds: A3Gb_-C-G G > A | cds | -C-G | % of motif variants that are | #motif_G > A_MC2/#motif |
| at MC2 motif % | | | G > A and at MC2 | |
| cds: A3Gc_C-C-GW C > T | cds | C-C-GW | % of motif variants ther are | #motif_C > T/#motif |
| motif % | | | C > T | |
| cds: A3Ge_SC-C-GS % | cds | SC-C-GS | % of all cds variants that are | #motif/#CDS |
| | | | motif variants | |
| cds: A3Gi_SG-C-G MC2% | cds | SG-C-G | % of motif variants that are at | #motif_MC2/#motif |
| | | | MC2 | |
| cds: A3Gi_SG-C-G non- | cds | SG-C-G | % of motif variants that | #motif_nonsyn/#motif |
| syn % | | | correspond to non-synonymous | |
| | | | changes | |
| cds: A3Gn_YYC-C-S C > | cds | YYC-C-S | % of C motif variants that are | #motif_C > T/#motif_Cstrand |
| T % | | | C > T | |
| cds: A3Gn_YYC-C-S C > | cds | YYC-C-S | % (of all cds variants) of | #motif_C > T_MC3/#CDS |
| T at MC3 cds % | | | C > T variants that are at MC3 | |
| cds: A3Go_TC-C-G MC1 | cds | TC-C-G | % of C > T motif variants that | #motif_MC1_nonsyn/#motif_MC1 |
| non-syn % | | | are non-synonymous | |
| cds: A3Bf_ST-C-G Ti % | cds | ST-C-G | % (of all cds variants) of motif | #motif_Ti/#CDS |
| | | | variants that are transitions | |
| cds: A3Bj_RT-C-G Ti % | cds | RT-C-G | % (of all cds variants) of motif | #motif_Ti/#CDS |
| | | | variants that are transitions | |
| g: A3Bj_RT-C-G C > T + | g | RT-C-G | % (of all genomic variants) of | #motif_Ti/#SNVs |
| G > A g % | | | motif variants that are | |
| | | | transitions | |
| cds: A3F_T-C- C > A % | cds | T-C- | % of C motif variants that are | #motif_C > A/#motif_Cstrand |
| | | | C > A | |
| cds: A3F_T-C- G > C % | cds | T-C- | % of G motif variants that are | #motif_G > C/#motif_Gstrand |
| | | | G > C | |
| g: A3F_T-C- Hits | g | T-C- | Number of motif variants | #motif |
| cds: A1_-C-A G > A at | cds | -C-A | % (of all cds variants) of | #motif_G > A_MC3/#CDS |
| MC3 cds % | | | G > A motif variants that are | |
| | | | at MC3 | |
| cds: 2Gen1_-C-C MC2% | cds | -C-C | % of motif variants that are | #motif_MC2/#motif |
| | | | at MC2 | |
| cds: 2Gen1_-C-C C > T | cds | -C-C | % of C > T motif variants that | #motif_C > T_MC1/#motif_C > T |
| at MC1% | | | are at MC1 | |
| cds: 2Gen1_-C-C C > | cds | -C-C | % of C motif variants that are | #motif_C > A/#motif_Cstrand |
| A % | | | C > A | |
| cds: 2Gen1_-C-C G > T | cds | -C-C | % of G > T motif variants that | #motif_G > T_MC1/#motif_G > T |
| at MC1% | | | are at MC1 | |
| cds: 2Gen1_-C-T MC3 | cds | -C-T | % of MC3 motif variants that | #motif_MC3_nonsyn/#motif_MC3 |
| non-syn % | | | are non-synonymous | |
| g: 2Gen1_-C-T % | g | -C-T | % of all genomic variants that | #motif/#SNVs |
| | | | are motif variants | |
| g: 2Gen1_-C-T C > G + | g | -C-T | % (of all genomic variants) of | (#motif_C > G + #motif_G > C)/#SNVs |
| G > C g % | | | motif variants that are C > G | |
| | | | or G > C | |
| nc: 2Gen1_-C-T C > A + | nc | -C-T | % (of all non-coding variants) | (#nc_motif_C > A + |
| G > T nc % | | | of motif variants that are | #nc_motif_G > T)/#nc_SNVs |
| | | | C > A or G > T | |
| cds: 2Gen2_G-C- Hits | cds | G-C- | Number of motif variants | #motif |
| cds: 2Gen2_A-C- MC3% | cds | A-C- | % of motif variants that are | #motif_MC3/#motif |
| | | | at MC3 | |
| nc: 2Gen2_A-C- C > | nc | A-C- | % (of all non-coding variants) | #nc_motif_Ti/#nc_SNVs |
| T + G > A nc % | | | of motif variants that are | |
| | | | transitions | |
| g: ADAR_2Gen1_-T-T A > | g | -T-T | % (of all genomic variants) of | (#motif_ A > T + #motif_T > A)/#motif |
| T + T > A % | | | motif variants that are A > T | |
| | | | or T > A | |
| cds: ADAR_2Gen2_A-T- | cds | A-T- | % of motif variants that are | #motif_A > C_MC1/#motif |
| A > C at MC1 motif % | | | A > C and MC1 | |
| cds: ADAR_2Gen2_T-T- % | cds | T-T- | % of all cds variants that are | #motif/#CDS |
| | | | motif variants | |
| cds: ADAR_2Gen2_G-T- | cds | G-T- | % of motif variants that are | #motif_MC2/#motif |
| MC2% | | | at MC2 | |
| g: ADAR_2Gen2_G-T- A > | g | G-T- | % (of all genomic variants) of | (#motif_ A > T + #motif_T > A)/#motif |
| T + T > A % | | | motif variants that are A > T | |
| | | | or T > A | |
| cds: 3Gen1_-C-CA Ti % | cds | -C-CA | % of all cds variants that are | #motif_Ti/#CDS |
| | | | transition motif variants | |
| cds: 3Gen1_-C-CA Ti | cds | -C-CA | % of transition motif variants | #motif_Ti_Cstrand/#motif_Ti |
| C:G % | | | that are on the C strand | |
| cds: 3Gen1_-C-CT C > | cds | -C-CT | % (of all cds variants) that | #motif_C > T_MC2/#CDS |
| T at MC2 cds % | | | are C > T MC2 motif variants | |
| cds: 3Gen1_-C-GT G > | cds | -C-GT | % of motif variants that are | #motif_G > A/#motif |
| A motif % | | | G > A | |
| cds: 3Gen1_-C-TC C > | cds | -C-TC | % of C > T motif variants that | #motif_C > T_MC3/#motif_C > T |
| T at MC3% | | | are at MC3 | |
| cds: 3Gen1_-C-TC C > | cds | -C-TC | % (of all cds variants) that | #motif_C > T/#CDS |
| T cds % | | | are C > T motif variants | |
| g: 3Gen1_-C-TC C > T + | g | -C-TC | % (of all genomic variants) | #motif_Ti/#SNVs |
| G > A g % | | | of motif variants that are | |
| | | | transitions | |
| cds: 3Gen1_-C-CC C > | cds | -C-CC | % of motif variants that are | #motif_C > T_MC1/#motif_C > T |
| T at MC1 motif % | | | C > T and MC1 | |
| cds: 3Gen1_-C-GC MC2% | cds | -C-GC | % of motif variants that are | #motif_MC2/#motif |
| | | | at MC2 | |
| cds: 3Gen1_-C-AG G | cds | -C-AG | % of G motif variants that are | #motif_Gstrand_Ti/#motif_Gstrand |
| Ti/Tv % | | | transitions | |
| cds: 3Gen1_-C-TG G | cds | -C-TG | % of G-strand motif variants | #motif_Gstrand_nonsyn/#motif_Gstrand |
| non-syn % | | | that correspond to non- | |
| | | | synonymous changes | |
| cds: 3Gen1_-C-CG G > | cds | -C-CG | % of G > A motif variants that | #motif_G > A_MC3/#motif_G > A |
| A at MC3% | | | are at MC3 | |
| cds: 3Gen2_T-C-T G > | cds | T-C-T | % of G > A motif variants that | #motif_G > A_MC2/#motif_G > A |
| A at MC2% | | | are at MC2 | |
| cds: 3Gen2_C-C-T MC3% | cds | C-C-T | % of motif variants that are | #motif_MC3/#motif |
| | | | at MC3 | |
| cds: 3Gen2_G-C-T % | cds | G-C-T | % of all cds variants that are | #motif/#CDS |
| | | | motif variants | |
| cds: 3Gen2_G-C-T C:G % | cds | G-C-T | % of motif variants that are | #motif_Cstrand/#motif |
| | | | on the C strand | |
| cds: 3Gen2_G-C-T C > | cds | G-C-T | % of motif variants that are | #motif_C > A/#motif |
| A motif % | | | C > A | |
| g: 3Gen2_G-C-T % | g | G-C-T | % of all genomic variants that | #motif/#SNVs |
| | | | are motif variants | |
| cds: 3Gen2_A-C-C MC2% | cds | A-C-C | % of motif variants that are | #motif_MC2/#motif |
| | | | at MC2 | |
| cds: 3Gen2_A-C-C non- | cds | A-C-C | % of motif variants that | #motif_nonsyn/#motif |
| syn % | | | correspond to non-synonymous | |
| | | | changes | |
| g: 3Gen2_A-C-C C > | g | A-C-C | % (of all genomic variants) of | (#motif_C > A + #motif_G > T)/#SNVs |
| A + G > T g % | | | motif variants that are C > | |
| | | | A or G > T | |
| cds: 3Gen2_T-C-C MC1% | cds | T-C-C | % of motif variants that are | #motif_MC1/#motif |
| | | | at MC1 | |
| cds: 3Gen2_T-C-C MC3% | cds | T-C-C | % of motif variants that are | #motif_MC3/#motif |
| | | | at MC3 | |
| cds: 3Gen2_C-C-C MC3% | cds | C-C-C | % of motif variants that are | #motif_MC3/#motif |
| | | | at MC3 | |
| cds: 3Gen2_A-C-G MC2 | cds | A-C-G | % of MC2 motif variants that | #motif_MC2_nonsyn/#motif_MC2 |
| non-syn % | | | are non-synomymous | |
| cds: 3Gen2_T-C-G Ti/Tv % | cds | T-C-G | % of motif variants that are | #motif_Ti/#motif |
| | | | transitions | |
| g: 3Gen2_T-C-G C > | g | T-C-G | % (of all genomic variants) | #motif_Ti/#SNVs |
| T + G > A g % | | | of motif variants that are | |
| | | | transitions | |
| cds: 3Gen3_AT-C- G > | cds | AT-C- | % of G > A motif variants | #motif_G > A_MC2/#motif_G > A |
| A at MC2% | | | that are at MC2 | |
| cds: 3Gen3_AT-C- C:G % | cds | AT-C- | % of motif variants that are | #motif_Cstrand/#motif |
| | | | on the C strand | |
| cds: 3Gen3_AG-C- MC2% | cds | AG-C- | % of motif variants that are | #motif_MC2/#motif |
| | | | at MC2 | |
| cds: 3Gen3_TT-C- C > | cds | TT-C- | % of motif variants that are | #motif_C > A_MC1/#motif_C > A |
| A at MC1 motif % | | | C > A and MC1 | |
| cds: 3Gen3_TG-C- G > | cds | TG-C- | % of G motif variants that | #motif_G > A/#motif_Gstrand |
| A % | | | are G > A | |
| cds: 3Gen3_TG-C- G | cds | TG-C- | % of G motif variants that | #motif_Gstrand_Ti/#motif_Gstrand |
| Ti/Tv % | | | are transitions | |
| g: 3Gen3_CA-C- C > | g | CA-C- | % (of all genomic variants) | (#motif_C > T + #motif_G > A)/#SNVs |
| T + G > A g % | | | of motif variants that are | |
| | | | transitions | |
| cds: 3Gen3_CT-C- MC3% | cds | CT-C- | % of motif variants that are | #motif_MC3/#motif |
| | | | at MC3 | |
| cds: 3Gen3_CT-C- G | cds | CT-C- | % of G motif variants that | #motif_Gstrand_nonsyn/#motif_Gstrand |
| non-syn % | | | are non-synonymous | |
| cds: 3Gen3_CT-C- C > | cds | CT-C- | % of motif variants that are | #motif_C > T_MC1/#motif_C > T |
| T at MC1 motif % | | | C > T and MC1 | |
| g: 3Gen3_GA-C- C > | g | GA-C- | % (of all genomic variants) | (#motif_C > A + #motif_G > T)/#SNVs |
| A + G > T g % | | | of motif variants that are | |
| | | | C > A or G > T | |
| cds: 3Gen3_GG-C- | cds | GG-C- | % of motif variants that | #motif_nonsyn/#motif |
| non-syn % | | | correspond to non-synonymous | |
| | | | changes | |
| cds: ADAR_3Gen1_-A-AT | cds | -A-AT | % (of all cds variants) of | #motif_Ti/#CDS |
| Ti % | | | motif variants that are | |
| | | | transitions | |
| cds: ADAR_3Gen1_-A-CA % | cds | -A-CA | % of all cds variants that | #motif/#CDS |
| | | | are motif variants | |
| cds: ADAR_3Gen1_- | cds | -A-CC | % (of all cds variants) that | #motif_A > G/#CDS |
| A-CC A > G cds % | | | are A > G motif variants | |
| cds: ADAR_3Gen2_C-A-C | cds | C-A-C | % (of all cds variants) of | #motif_T > G_MC3/#CDS |
| T > G at MC3 cds % | | | T > G variants and at | |
| | | | MC3 | |
| cds: ADAR_3Gen2_G-A-C | cds | G-A-C | % of motif variants that | #motif_nonsyn/#motif |
| non-syn % | | | correspond to non-synonymous | |
| | | | changes | |
| cds: ADAR_3Gen3_AC-A- | cds | AC-A- | % (of all cds variants) that | #motif_A > G/#CDS |
| A > G cds % | | | are A > G motif variants | |
| cds: ADAR_3Gen3_CA-A- | cds | CA-A- | % (of all cds variants) of | #motif_Ti/#CDS |
| Ti % | | | motif variants that are | |
| | | | transitions | |
| cds: ADAR_3Gen3_CT-A- | cds | CT-A- | % of motif variants that | #motif_A > G/#motif |
| A > G motif % | | | are A > G | |
| cds: 4Gen3_AC-C-T | cds | AC-C-T | % of motif variants that | #motif_Ti/#motif |
| Ti/Tv % | | | are transitions | |
| cds: 4Gen3_AG-C-T | cds | AG-C-T | % of MC1 motif variants that | #motif_MC1_nonsyn/#motif_MC1 |
| MC1 non-syn % | | | are non-synonymous | |
| cds: 4Gen3_AG-C-T | cds | AG-C-T | % of motif variants that are | #motif_G > A_MC1/#motif |
| G > A at MC1 motif % | | | G > A and MC1 | |
| cds: 4Gen3_CA-C-C % | cds | CA-C-C | % of all cds variants that | #motif/#CDS |
| | | | are motif variants | |
| cds: 4Gen3_CA-C-C MC1% | cds | CA-C-C | % of motif variants that are | #motif_MC1/#motif |
| | | | at MC1 | |
| cds: 4Gen3_CT-C-C C > | cds | CT-C-C | % of C > T motif variants | #motif_C > T_MC1/#motif_C > T |
| T at MC1% | | | that are at MC1 | |
| cds: 4Gen3_GC-C-A % | cds | GC-C-A | % of all cds variants that | #motif/#CDS |
| | | | are motif variants | |
| cds: 4Gen3_GC-C-C C > | cds | GC-C-C | % of C > T motif variants | #motif_C > T_MC2/#motif_C > T |
| T at MC2% | | | that are at MC2 | |
| g: 4Gen3_GG-C-G C > | g | GG-C-G | % (of all genomic variants) | #motif_Ti/#SNVs |
| T + G > A g % | | | of motif variants that are | |
| | | | C > T or G > A | |
| cds: 4Gen3_TA-C-C | cds | TA-C-C | % of motif variants that | #motif_nonsyn/#motif |
| non-syn % | | | correspond to non-synonymous | |
| | | | changes | |
| cds: 4Gen3_TG-C-T | cds | TG-C-T | % of transition motif | #motif_Ti_Cstrand/#motif_Ti |
| Ti C:G % | | | variants that are on the C | |
| | | | strand | |
| cds: 4Gen3_TT-C-C % | cds | TT-C-C | % of all cds variants that | #motif/#CDS |
| | | | are motif variants | |
| cds: 4Gen3_TT-C-T % | cds | TT-C-T | % of all cds variants that | #motif/#CDS |
| | | | are motif variants | |
| g: ADAR_4Gen3_AG-A-G | g | AG-A-G | % (of all genomic variants) | (#motif_A > C + #motif_T > G)/#SNVs |
| A > C + T > G % | | | of motif variants that are | |
| | | | A > C or T > G | |

In some instances, the metrics set forth in Table D have one or more related metric(s). A related metric as used herein is one that can be used a proxy for another metric in the methods of the disclosure. Related metrics typically represent the same type or similar information the metric to which it is related.

For example, metrics can be related when one metric corresponds to a subset of another metric. Non-limiting examples, include motif metrics that are a subset of other motif metrics, e.g. CT-C-A SNVs are a subset of T-C-A SNVs, and are therefore related; and G-G-metrics are a subset of “All G” metrics, and are therefore related.

In other examples, metrics that encompass an assessment of codon context may be related, e.g. MC1% metrics are related to MC2% and MC3% as the sum of all MC1%, MC2% and MC3% metrics is 100%. Thus, for example, cds:4Gen3_CA-C-C MC1% is related to cds:4Gen3_CA-C-C MC2% and cds:4Gen3_CA-C-C MC3%.

In further examples, mutation type metrics may be related, e.g. C>T metrics may measure the proportion of C>T SNVs as a percentage of all SNVs, all SNVs in the coding region, all SNVs within a specific motif, or C-strand motif SNVs. Consequently, C>A % is related to C>T % and C>G %.

In other examples, G and C strand metrics may be related. For example, C-strand and G-strand motif metrics are a subset of motif-related metrics, e.g. Motif G-strand MC1% is related to Motif MC1%; and Motif C-strand Ti % is related to Motif Ti %.

In other examples, “motif Ti %” metric, which is a measure of transition SNVs of the motif, is a subset of “motif %” which counts all motif SNVs. Consequently, motif Ti % and motif % are related metrics.

In further examples, percentage metrics are related to Hit/Count metrics, as these are calculated by divided Hits/Counts by a denominator such as, for example, all SNVs, all SNVs in the coding region, all SNVs within a specific motif, or all C-strand motif SNVs.

In other examples, CDS, non-coding and genomic region metrics may be related. For example, non-coding SNVs are a subset of genomic SNVs and are therefore relate; and CDS SNVs are a subset of genomic SNVs, and therefore count based and transition/transversion metrics are related.

In further examples, non-synonymous metrics are related to MC1, MC2 and MC3 percentages, as MC3 mutations are less likely to encode a non-synonymous amino acid change and MC1 and MC2 SNVs are more likely to encode non-synonymous amino acid changes.

In other examples, metrics based on the same count but use a different denominator are related. For example, motif C>A SNVs can be represented as a percentage of C-strand motif SNVs, all motif SNVs or all CDS SNVs, and consequently each is related.

In further examples, all “primary” motif metrics are related to other metrics of AID, ADAR, APOBEC3G and APOBEC3B as primary motif metrics relate to the sum of these four motifs.

In other examples, all “other” motif metrics are a subset of “All” metrics and are therefore related, e.g. All G SNVs=Other G SNVs (G SNVs not at a primary deaminase motif)+Primary G SNV (i.e. G SNVs at a primary deaminase motif).

Based on the above, those skilled in the art would be able to determine what metrics may be related to those set forth in Table D. In non-limiting examples, the metric g:CG total, which is a calculation of the number of variants at C or G in the genome, has multiple related metrics that represent same type or similar information, including, for example, total variants in VCF, total SNVs in VCF, g:variant total, cds:CDS Variants, CDS total, cds:All G total, cds:All C total, cds:Other G total, aa synonymous, cds:Other C total, aa non-synonymous.

In another example, related metrics for g:A3Bj_RT-C-G C>T+G>A g % include cds:A3F_T-C-MC1%, cds:3Gen3_TC-C-%, cds:3Gen2_T-C-G C:G %, g:3Gen2_T-C-G C>T+G>A %, g:3Gen2_T-C-G C>T+G>A g %, cds:3Gen2_T-C-G C>T %, cds:3Gen2_T-C-G C>T motif %, and cds:3Gen2_T-C-G C>T cds %.

In a further example, related metrics for g:A3F_T-C-Hits include cds:A3F_T-C-MC3 non-syn %, cds:A3F_T-C-Hits, g:A3B_T-C-W Hits, g:3Gen3_CT-C-Hits, cds:3Gen3_TT-C-G non-syn %, cds:A3B_T-C-W Hits, g:3Gen3_TT-C-Hits, g:A3Gh_S-C-GS Hits, g:A3B_T-C-W %, cds:3Gen2_T-C-T G non-syn %, g:3Gen3_AT-C-Hits, cds:A3B_T-C-W MC3 non-syn %, nc:3Gen3_CT-C-%, g:3Gen2_T-C-A Hits, cds:3Gen2_T-C-G G non-syn %, cds:3Gen3_AT-C-Hits, nc:3Gen3_CT-C-Hits, cds:3Gen2_T-C-G MC1 non-syn % and g:3Gen2_T-C-T Hits.

### 4. Assessing a Nucleic Acid Molecule for SNVs

Any method known in the art for obtaining and assessing the sequence of a nucleic acid molecule can be used in accordance with the methods and systems of the present disclosure. The nucleic acid molecule analyzed using the systems and methods of the present disclosure can be any nucleic acid molecule, although is generally DNA (including cDNA). Typically, the nucleic acid is mammalian nucleic acid, such as human nucleic acid.

The nucleic acid can be obtained from any biological sample. The biological sample may comprise a bodily fluid, tissue or cells. In particular examples, the biological sample is a bodily fluid, such as saliva or blood. In other examples, the biological sample is a tissue biopsy. A biological sample comprising tissue or cells may from any part of the body and may comprise any type of cells or tissue. Typically, the sample comprises cancer or tumour cells. Consequently, in some examples, the sample is from a particular region or location in a subject in which the cancer or tumour is present, and thus comprises, for example, breast, prostate, liver, colon, stomach, pancreatic, skin, thyroid, cervical, lymphoid, haematopoietic, bladder, lung, renal, rectal, ovarian, uterine, and head or neck tissue or cells. In particular examples, the biological sample used to detect the likelihood of progression or recurrence of a cancer is matched to the type of cancer. By way of an illustration, is the subject suffers from or has suffered from an ovarian cancer, then the sample is derived from ovarian tissue or cells.

The nucleic acid molecule can contain a part or all of one gene, or a part or all of two or more genes. Most typically, the nucleic acid molecule comprises the whole genome or whole exome, and it is the sequence of the whole genome or whole exome that is analyzed in the methods of the disclosure. In instances where the whole genome or whole exome is used for analysis, SNVs that are in coding regions, non-coding regions or all regions (referred to as “genome”) may be assessed.

When performing the methods of the present disclosure, the sequence of the nucleic acid molecule may have been predetermined. For example, the sequence may be stored in a database or other storage medium, and it is this sequence that is analyzed according to the methods of the disclosure. In other instances, the sequence of the nucleic acid molecule must be first determined prior to employment of the methods of the disclosure. In particular examples, the nucleic acid molecule must also be first isolated from the biological sample. Thus, in some embodiments, the methods of the present disclosure comprise a step of obtaining a biological sample from a subject, optionally isolating nucleic acid from the sample, sequencing the nucleic acid and then analysing the nucleic acid so as to detect SNVs, as described herein. In other embodiments, the biological sample has already been obtained from the subject, and the methods comprises a step of isolating the nucleic acid, sequencing the nucleic acid and then analysing the nucleic acid so as to detect SNVs. In further embodiments, the biological sample has already been obtained from the subject and the nucleic acid has already been isolated, and the methods comprises a step of sequencing the nucleic acid and then analysing the nucleic acid so as to detect SNVs. In still further embodiment, the biological sample has already been obtained from the subject and the nucleic acid has already been isolated and sequenced, before the methods of the present disclosure are performed.

Methods for obtaining nucleic acid and/or sequencing the nucleic acid are well known in the art, and any such method can be utilized for the methods described herein. In some instances, the methods include amplification of the isolated nucleic acid prior to sequencing, and suitable nucleic acid amplification techniques are well known to a person of ordinary skill in the art. Nucleic acid sequencing techniques are well known in the art and can be applied to single or multiple genes, or whole exomes, transcriptomes or genomes. These techniques include, for example, capillary sequencing methods that rely upon ‘Sanger sequencing’ (Sanger et al. (1977) Proc Natl Acad Sci USA 74: 5463-5467) (i.e., methods that involve chain-termination sequencing), as well as “next generation sequencing” techniques that facilitate the sequencing of thousands to millions of molecules at once. Such methods include, but are not limited to, pyrosequencing, which makes use of luciferase to read out signals as individual nucleotides are added to DNA templates; “sequencing by synthesis” technology (Illumina), which uses reversible dye-terminator techniques that add a single nucleotide to the DNA template in each cycle; and SOLiD™ sequencing (Sequencing by Oligonucleotide Ligation and Detection; Life Technologies), which sequences by preferential ligation of fixed-length oligonucleotides. These next generation sequencing techniques are particularly useful for sequencing whole exomes and genomes. Other exemplary sequencing platforms include third generation (or long-read) sequencing platforms, such as single-molecule nanopore sequencing using the MiniION™ or GridION™ sequencers (developed by Oxford Nanopore and involving passing a DNA molecule through a nanoscale pore structure and then measuring changes in electrical field surrounding the pore), or single molecule real time sequencing (SMRT) utilizing a zero-mode waveguide (ZMW), such as developed by Pacific Biosciences.

Once the sequence of the nucleic acid molecule is obtained, SNVs are then identified. SNVs may be identified by comparing the sequence to a reference sequence. The reference sequence may be the sequence of a nucleic acid molecule from a database, such as reference genome. In particular examples, the reference sequence is a reference genome, such as GRCh38 (hg38), GRCh37 (hg19), NCBI Build 36.1 (hg18), NCBI Build 35 (hg17) and NCBI Build 34 (hg16). In some embodiments, the SNVs are reviewed to remove known single nucleotide polymorphisms (SNPs) from further analysis, such as those identified in the various SNP databases that are publicly available. In further embodiments, only those SNVs that are within a coding region of an ENSEMBL gene are selected for further analysis. In addition to identifying the SNVs, the codon containing the SNV and the position of the SNV within the codon (MC-1, MC-2 or MC-3) may be identified. Nucleotides in the flanking 5′ and 3′ codons may also be identified so as to identify the motifs. In some instances of the methods of the present disclosure, the sequence of the non-transcribed strand (equivalent to the cDNA sequence) of the nucleic acid molecules is analyzed. In other instances, the sequence of the transcribed strand is analyzed. In further instances, the sequences of both strands are analyzed.

Having identified one or more SNVs in a nucleic acid molecule, one or metrics (or CPAS), can be determined by making the appropriate calculations, as set forth above.

### 5. Kits and Systems for Detecting SNVs and Determining Metrics

All the essential materials and reagents required for detecting SNVs may be assembled together in a kit. For example, when the methods of the present disclosure include first isolating and/or sequencing the nucleic acid to be analyzed, kits comprising reagents to facilitate that isolation and/or sequencing are envisioned. Such reagents can include, for example, primers for amplification of DNA, polymerase, dNTPs (including labelled dNTPs), positive and negative controls, and buffers and solutions. Such kits will also generally comprise, in suitable means, distinct containers for each individual reagent. The kit can also feature various devices, and/or printed instructions for using the kit.

In some embodiments, the methods described generally herein are performed, at least in part, by a processing system, such as a suitably programmed computer system. For example, a processing system can be used to analyze the nucleic acid sequence, identify SNVs, and/or determine metrics. A stand-alone computer, with the microprocessor executing applications software allowing the above-described methods to be performed, may be used. Alternatively, the methods can be performed, at least in part, by one or more processing systems operating as part of a distributed architecture. For example, a processing system can be used to identify SNV types, the codon context of an SNV and/or motifs within one or more nucleic acid sequences so as to generate the metrics described herein. In some examples, commands inputted to the processing system by a user assist the processing system in making these determinations.

In one example, a processing system includes at least one microprocessor, a memory, an input/output device, such as a keyboard and/or display, and an external interface, interconnected via a bus. The external interface can be utilised for connecting the processing system to peripheral devices, such as a communications network, database, or storage devices. The microprocessor can execute instructions in the form of applications software stored in the memory to allow the methods of the present disclosure to be performed, as well as to perform any other required processes, such as communicating with the computer systems. The applications software may include one or more software modules, and may be executed in a suitable execution environment, such as an operating system environment, or the like.

### 6. Systems for Generating Progression Indicators

The present disclosure also provides systems and processes for generating a progression indicator for assessing the likelihood that a cancer will progress or recur.

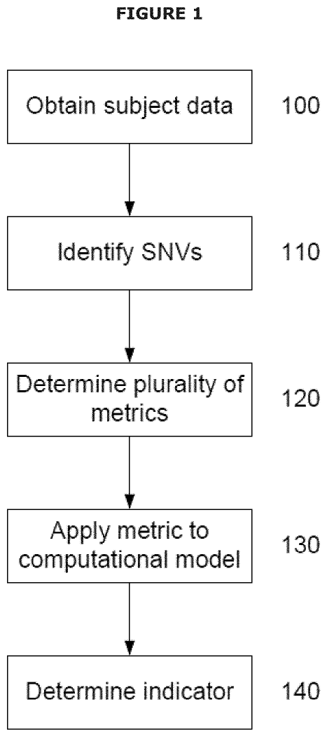

An example of the process for generating a progression indicator for assessing the likelihood that a cancer will progress or recur will now be described with reference to FIG. 1.

For the purpose of this example, it is assumed that the method is performed at least in part using one or more electronic processing devices typically forming part of one or more processing systems, such as servers, personal computers or the like and which may optionally be connected to one or more processing systems, data sources or the like via a network architecture as will be described in more detail below.

For the purpose of explanation, the term “reference subject” is used to refer to one or more individuals in a sample population, with “reference subject data” being used to refer to data collected from the reference subjects. The term “subject” refers to any individual that is being assessed for the purpose of determining a likelihood of cancer progression or recurrence, with “subject data” being used to refer to data collected from the subject. The reference subjects and subjects are mammals, and more particularly humans, although this is not intended to be limiting and the techniques could be applied more broadly to other vertebrates and mammals.

In this example, at step 100 subject data is obtained which is at least partially indicative of a sequence of a nucleic acid molecule from the subject. The subject data could be obtained in any appropriate manner, as described above, such as, for example, whole exome sequencing or whole genome sequencing of a biological sample from a subject.

The subject data may also include additional data, such as data regarding subject attributes or other physiological signals measured from the subject, such as measures of physical or mental activity, or the like, as will be described in more detail below.

At step 110 the subject data is analysed to identify SNVs within the nucleic acid molecule, as described above.

At step 120 the identified SNVs are used to determine a plurality of metrics, such at least 5, 10, 15, 20, 25, 30, 40, 50, 60, 70, 80, 90, 100, 120, 130 or 140 of those set forth in Table D or a related metric to one set forth in Table D. The metrics used may vary depending upon a range of factors, such as the computational model to be used, subject attributes, the particular type of cancer being assessed, or the like, as will be described in more detail below.

At step 130 the two or more metrics are applied to one or more computational models. The computational model(s) typically embody relationship between cancer progression or recurrence and the plurality of metrics, and can be obtained by applying one or more analytical techniques, such as machine learning, conventional clustering, linear regression or Bayesian methods, or any of the other techniques known in the art or described below, to reference metrics derived from a plurality of reference metrics obtained from reference subjects having a known cancer progression or recurrence.

Thus, it will be appreciated that in practice reference subject data, equivalent to subject data, is collected for a plurality of reference subjects having a different cancer progression or recurrence. The collected reference subject data is used to calculate reference metrics, which are then used to train the computational model(s) so that the computational model(s) can discriminate between different progression or recurrence, based on metrics derived from the subject's SNVs. The nature of the computational model will vary depending on the implementation and examples will be described in more detail below.

The computational model is used to determine a progression indicator which is indicative of the likelihood of cancer progression or recurrence at step 140, i.e. the progression indicator is indicative of whether or not the subject has a cancer that is likely progress or recur. This allows a supervising clinician or other medical personnel to assess an appropriate therapy or intervention for the subject.

In one example, the progression indicator could include a numerical value, for example indicating that there is a 60%, 70%, 80%, 90%, or 95% chance the subject has a cancer that is likely to progress or recur (or put another way, there is a 60%, 70%, 80%, 90%, or 95% chance that the cancer in a subject will progress or recur). However, this is not necessarily essential, and it will be appreciated that any suitable form of indicator could be used.

Accordingly, it will be appreciated that the above described method utilises an analytical technique such as a machine learning technique in order to assess cancer progression or recurrence utilising certain defined metrics.

In one example, the particular metrics are used in a variety of combinations in order to provide computational models having a discriminatory performance, such as an accuracy, sensitivity, specificity or area under the receiver characteristic operating curve (AUROC) of greater than 70%.

The above described approach provides a mechanism for objectively assessing the likelihood of a subject's cancer progressing or recurring, which can assist in identifying the most effective therapy and/or the need for therapy.

A number of further features will now be described.

In one example, the motif metric group comprises at least 5, 10, 15, 20, 25, 30, 40, 50, 60, 70, 80, 90, 100, 120, 130 or 140 metrics selected from those set forth in Table D and those related to the metrics set forth in Table D.

The system can use a number of different combinations of computational models, for example depending on the particular discriminatory abilities of the models and the particular cancer therapies of interest.

In one example, the system uses multiple different computational models, which can improve the ability to accurately assess cancer progression or recurrence. In this instance, the processing devices apply respective metrics to respective models to determine individual scores, which are then aggregated to determine a progression indicator.

The nature of the model will vary depending on the implementation, and on example the model could include a decision tree or similar, and in one preferred example, multiple decision trees are used, with results being aggregated. However, it will be appreciated that this is not essential, and other models could be used.

As previously mentioned, multiple metrics are used in order to increase the accuracy of the computational model(s), with these typically being selected from across the groups, in order to maximise the effectiveness of the discriminatory performance of the computational model(s).

In general the number of metrics used will vary depending on the implementation and the outcome of training. In one example, at least 5, 10, 15, 20, 25, 30, 40, 50, 60, 70, 80, 90, 100, 120, 130 or 140 metrics selected from those set forth in Table D and those related to the metrics set forth in Table D are used. Optionally, additional metrics, such as any described in WO2019095017 are used.

The analysis may also be performed to take into account subject attributes, such as subject characteristics, possible medical conditions suffered by the subject, possible interventions performed, or the like. In this example, the one or more processing devices can use the one or more subject attributes to apply the computational model so that the metrics are assessed based on reference metrics derived for one or more reference subjects having similar attributes to the subject attributes. This can be achieved in a variety of ways, depending on the preferred implementation, and can include selecting metrics and/or one of a number of different computational models at least in part depending on the subject attributes. Irrespective of how this is achieved, it will be appreciated that taking into account subject attributes can further improve the discriminatory performance by taking into account that subjects with different attributes may have differing cancer progression or recurrence.

The subject attributes could include subject characteristics such as a subject age, height, weight, sex or ethnicity, body states, such as a healthy or unhealthy body states or one or more disease states, such as whether the subject is obese. The subject attributes could include one or more medical symptoms, such as an elevated temperature, heart rate, or blood pressure, whether the subject is suffering from nausea, or the like. Finally, the subject attributes could include dietary information, such as details of any food or drink consumed, or medication information, including details of any medications taken either as part of a medication regimen or otherwise.

The subject attributes could be determined in any one of a number of ways, for example by way of a clinical assessment, by querying a patient medical record, based on user input commands, or by receiving sensor data from a sensor, such as a weight or heart activity sensor, or the like.

In one example, the one or more processing devices display a representation of the progression indicator, store the progression indicator for subsequent retrieval or provide the progression indicator to a client device for display. Thus, it will be appreciated that the progression indicator can be used in a variety of manners, depending on the preferred implementation.

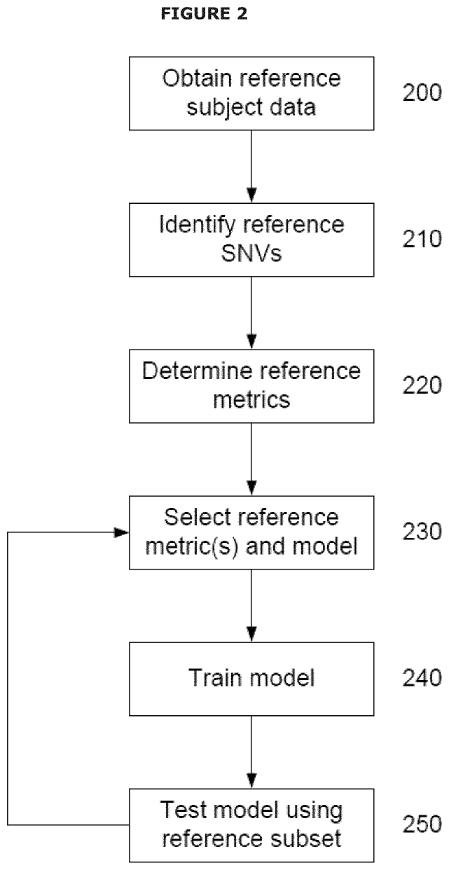

The above described approaches use one or more computational models in order to determine a progression indicator, and an example of a process for generating such model(s) will now be described with reference to FIG. 2.

In this example, reference subject data is obtained at step 200, which is indicative of a sequence of a nucleic acid molecule from the reference subject, as well as cancer progression or recurrence (or non-progression or -recurrence). At step 210 the reference subject data is analysed to identify SNVs within the nucleic acid molecule. At step 220 the reference subject data is analysed to determine reference metrics.

Steps 200 to 220 are largely analogous to steps 100 to 120 described with respect to obtaining and analysing subject data of a subject, and it will therefore be appreciated that these can be performed in a largely similar manner, and hence will not be described in further detail.

In contrast to subject data however, as the reference subject data is used in training a computational model, it is typically to determine reference metrics for all available metrics, rather than just selected ones of the metrics, allowing this to be used in order to ascertain which of the metrics are most useful in discriminating between individuals that likely to have cancer progression or recurrence.

At step 230 a combination of the reference metrics and one or more generic computational models are selected, with the reference metrics and cancer progression or recurrence (or non-progression or recurrence) being used to train the model at step 240. The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail.

Accordingly, the above described process provides a mechanism to develop a computational model that can be used in generating a progression indicator using the process described above with respect to FIG. 1.

In addition to simply generating the model, the process typically includes testing the model at step 250 to assess the discriminatory performance of the trained model. Such testing is typically performed using a subset of the reference subject data, and in particular, different reference subject data to that used to train the model, to avoid model bias. The testing is used to ensure the computational model provides sufficient discriminatory performance. In this regard, the discriminatory performance is typically based on an accuracy, sensitivity, specificity and AUROC, with a discriminatory performance of at least 70% being required in order for the model to be used.

It will be appreciated that if the model meets the discriminatory performance, it can then be used in determining a progression indicator using the process outlined above with respect to FIG. 1. Otherwise, the process returns to step 230 allowing different metrics and/or models to be selected, with training and testing then being repeated as required.

Thus, in one example, the one or more processing devices select a plurality of reference metrics, typically selected as a subset of each of the available metrics listed above, train one or more computational models using the plurality of reference metrics, test the computational models to determine a discriminatory performance of the model(s) and if the discriminatory performance of the model(s) falls below a threshold then selectively retrain the computational model(s) using a different plurality of reference metrics and/or a plurality of metrics from different reference subject data and/or train different computational model(s). Accordingly, it will be appreciated that the above described process can be performed iteratively utilising different metrics and/or different computational models until a required degree of discriminatory power is obtained.

Thus, in one example, the one or more processing devices train the model using at least 20, 40, 60, 80, 100, 200, 400, 600, 800, 1000, 2000 or more metrics, with the resulting models typically using significantly less metrics, such as less than 100.

Additionally and/or alternatively, the one or more processing devices can select a plurality of combinations of reference metrics, train a plurality of computational models using each of the combinations, test each computational model to determine a discriminatory performance of the model and select one or more of the computational models with the highest discriminatory performance for use in determining a progression indicator.

In addition to using the metrics to train the models, the training can also be performed taking into account reference subject attributes, so that models are specific to respective reference subject attributes or can take the subject attributes into account when determining the likelihood of cancer progression or recurrence. In one example, this process involves having the one or more processing devices perform clustering using the using the reference subject attributes to determine clusters of reference subjects having similar reference subject attributes, for example using a clustering technique such as k-means clustering, and then training the computational model at least in part using the reference subject clusters. For example clusters of reference individuals suffering from a particular form of cancer could be identified, with this being used to train a computational model to identify likely progression or recurrence.

Accordingly, the above described techniques provide a mechanism for training one or more computational models to determine the likelihood of cancer progression or recurrence using a variety of different metrics, and then using the model(s) to generate progression indicators indicative of the likelihood of cancer progression or recurrence.

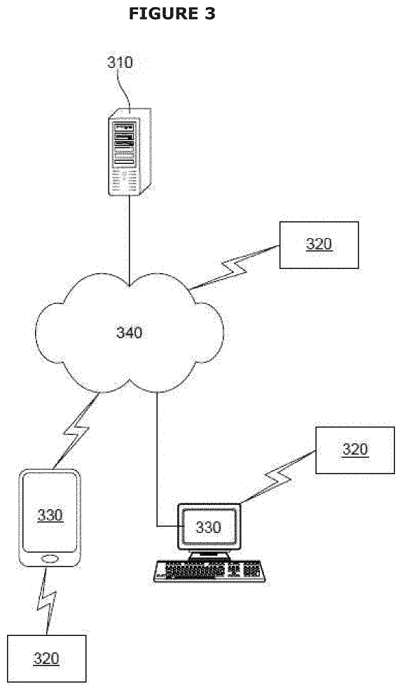

An example of a monitoring system will now be described in more detail with reference to FIG. 3.

In this example, one or more processing systems 310 are provided coupled to one or more client devices 330, via one or more communications networks 340, such as the Internet, and/or a number of local area networks (LANs). A number of sequencing devices 320 are provided, with these optionally being connected directly to the processing systems 310 via the communications networks 340, or more typically, with these being coupled to the client devices 330.

Any number of processing systems 310, sequencing devices 320 and client devices 330 could be provided, and the current representation is for the purpose of illustration only. The configuration of the networks 340 is also for the purpose of example only, and in practice the processing systems 310, sequencing devices 320 and client devices 330 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

In this example, the processing systems 310 are adapted to receive and analyse subject data received from the sequencing devices 320 and/or client devices 330, allowing computational models to be generated and used to determine progression indicators, which can then be displayed via the client devices 330. Whilst the processing systems 310 are shown as single entities, it will be appreciated they could include a number of processing systems distributed over a number of geographically separate locations, for example as part of a cloud based environment. Thus, the above described arrangements are not essential and other suitable configurations could be used.

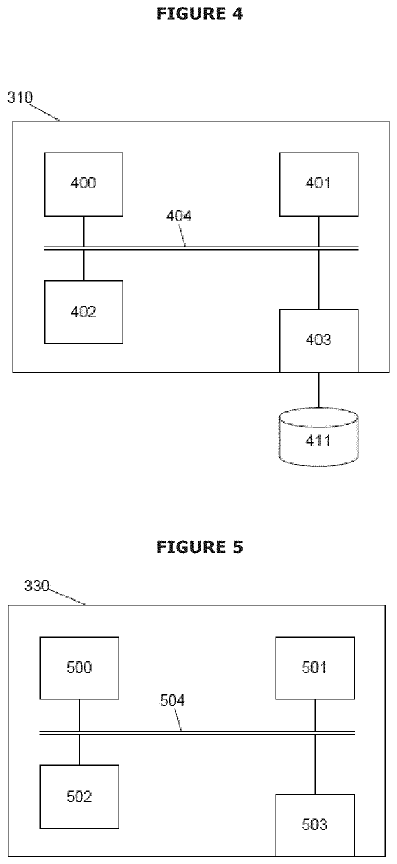

An example of a suitable processing system 310 is shown in FIG. 4. In this example, the processing system 310 includes at least one microprocessor 400, a memory 401, an optional input/output device 402, such as a keyboard and/or display, and an external interface 403, interconnected via a bus 404 as shown. In this example the external interface 403 can be utilised for connecting the processing system 310 to peripheral devices, such as the communications networks 340, databases 411, other storage devices, or the like. Although a single external interface 403 is shown, this is for the purpose of example only, and in practice multiple interfaces using various methods (eg. Ethernet, serial, USB, wireless or the like) may be provided.

In use, the microprocessor 400 executes instructions in the form of applications software stored in the memory 401 to allow the required processes to be performed. The applications software may include one or more software modules, and may be executed in a suitable execution environment, such as an operating system environment, or the like.

Accordingly, it will be appreciated that the processing system 310 may be formed from any suitable processing system, such as a suitably programmed PC, web server, network server, or the like. In one particular example, the processing system 310 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

As shown in FIG. 5, in one example, the client device 330 includes at least one microprocessor 500, a memory 501, an input/output device 502, such as a keyboard and/or display, an external interface 503, interconnected via a bus 504 as shown. In this example the external interface 503 can be utilised for connecting the client device 330 to peripheral devices, such as the communications networks 340, databases, other storage devices, or the like. Although a single external interface 503 is shown, this is for the purpose of example only, and in practice multiple interfaces using various methods (eg. Ethernet, serial, USB, wireless or the like) may be provided. The card reader 504 can be of any suitable form and could include a magnetic card reader, or contactless reader for reading smartcards, or the like.

In use, the microprocessor 500 executes instructions in the form of applications software stored in the memory 501, and to allow communication with one of the processing systems 310 and/or sequencing devices 320.

Accordingly, it will be appreciated that the client device 330 be formed from any suitably programmed processing system and could include suitably programmed PCs, Internet terminal, lap-top, or hand-held PC, a tablet, a smart phone, or the like. However, it will also be understood that the client device 330 can be any electronic processing device such as a microprocessor, microchip processor, logic gate configuration, firmware optionally associated with implementing logic such as an FPGA (Field Programmable Gate Array), or any other electronic device, system or arrangement.

Examples of the processes for generating progression indicators will now be described in further detail. For the purpose of these examples it is assumed that one or more respective processing systems 310 are servers adapted to receive and analyse subject data, and generate and provide access to progression indicators. The servers 310 typically execute processing device software, allowing relevant actions to be performed, with actions performed by the server 310 being performed by the processor 400 in accordance with instructions stored as applications software in the memory 401 and/or input commands received from a user via the I/O device 402. It will also be assumed that actions performed by the client devices 330, are performed by the processor 500 in accordance with instructions stored as applications software in the memory 501 and/or input commands received from a user via the I/O device 502.

However, it will be appreciated that the above described configuration assumed for the purpose of the following examples is not essential, and numerous other configurations may be used. It will also be appreciated that the partitioning of functionality between the different processing systems may vary, depending on the particular implementation.

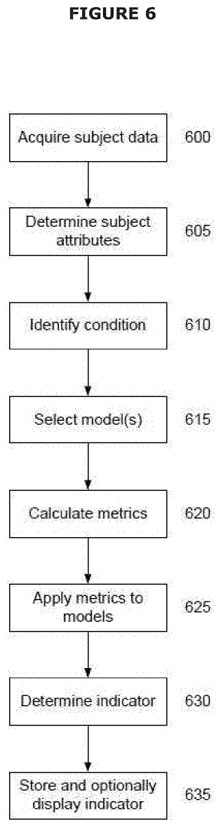

An example of the process for analysing subject data for an individual will now be described in more detail with reference to FIG. 6.

In this example, at step 600 the server 310 obtains subject data, either retrieving this from a stored record or receiving this from a sequencing device, optionally via a client device 330, depending upon the preferred implementation.

At step 605, the server 310 determines subject attributes, for example by retrieving these from a database, or obtaining these as part of the subject data. The subject attributes can be used for selecting one or more computational models to be used and/or may be combined with the metrics in order to allow the computational model(s) to be applied. In this regard, the metrics for the subject are typically analysed based on reference metrics for reference subjects having similar attributes to the subject. This could be achieved by using different computational models for different combinations of attributes, or by using the attributes as inputs to the computational model.

At step 610, the server 310 determines a cancer type of the cancer suffered by the subject, using this to select one or more computational models at step 615. In this regard, different computational models will typically be used to assess likelihood of progression or recurrence for different types of cancer.

Having selected a model, at step 620, the server 310 then calculates the relevant metrics required by the model.

At step 625 metrics are applied to the computational model(s), for example by using the relevant metrics, optionally together with one or more subject attributes, to perform a decision tree assessment, resulting in the generation of an indicator that is indicative of likelihood of cancer progression or recurrence at step 630.

At step 635, the server 310 stores the progression indicator, typically as part of the subject data, optionally allowing the progression indicator to be displayed, for example by forwarding this to the client device for display.

A specific example of a machine learning approach will now be described in more detail.

In this example, sequencing data are run through the above described process and metrics of interest are identified and quantified with these being collated patient to build a profile.

This is then used to identify patient profiles that are, for example, “High PFS” (e.g. those who achieve a particular time period in which the cancer does not progress), and “Low PFS” (e.g. those who do not achieve a particular time period in which the cancer does not progress, or to re-phrase, those in whom the cancer has progressed within a particular time period). There are many ways the data can be analysed and the following approach described herein is tailored to cancer progression.

Initially, the sequence data is collected and used to produce metrics for each patient. The raw results can be exported and analysed by cleaning the data (e.g. metadata not required for analysis are removed) before patients are grouped for analysis.

To prove effectiveness of the process, a number of cancer patients have been analysed, with the patients being grouped into three categories: training data, tuning data and validation data. The training and tuning datasets are comprised of a large number of patients, with patients split into each group randomly; the validation dataset is comprised of patients whose data was not including the training and tuning datasets.

A typical experimental approach is to ‘set aside’ the validation dataset (the data being predicted) and collate the rest of the patients together. The collated patients are then split 75:25 (with an ˜equal proportion of Responders/Non-Responders) into training (˜75%) and tuning (˜25%) datasets.

Once the data are grouped, High PFS and Low PFS can be plotted for each metric for patients in the validation dataset. Plotting the data provides a method for further investigating metrics identified by the machine learning analysis as being important, although isn't directly involved in any of the calculations/analyses.

Once the data are grouped and formatted appropriately, the machine learning algorithm is applied to generate the computational model. In one example, the algorithm used is XGBoost, which is an implementation of ‘gradient boosting decision trees’, which are specifically designed for speed and performance on large datasets (millions of data points).

The approach calculates a large number of decision trees and checks each decision tree to find the one that maximizes the predictive score on the training dataset. The predictive model can then be applied for predictive purposes. In practice, the preferred approach uses an ‘ensemble’ of decision trees, each using different combinations of metrics, to make predictions, thereby increasing accuracy.

This approach can be very computationally expensive and can result in many millions of possible trees and many possible ensembles. In general, to optimise this approach each model is trained using a subset of metrics, typically >100 in each case, and whilst a single metric could be used, in practice there are generally >10, >20, or >30 for models with a reasonable level of accuracy.

There are a number of parameters that can be adjusted when building the XGBoost model and so multiple passes can be conducted to optimize settings, with the optimized settings then being used. The optimisation is conducted without human interference (various combinations of settings are tested and the computer identifies which settings are optimal) making this approach consistent, reproducible, and minimises susceptibility to experimenter bias.

Once the model is built, tuned and applied to the data, it is possible to determine which metrics were important for the predictions made. The contribution of each metric to the overall prediction is cumulative, with the score for specific variables contributing in a ‘weighted’ manner to the overall prediction (i.e. the score for one metric may indicate the subject is responder, but the score for another metric may indicate that the subject is not a responder).

Using this machine learning approach, patient outcome can be predicted with good accuracy when applied to ‘real world’ datasets (see Examples 2 and 3).

### 7. Diagnostic and Therapeutic Applications

Using the methods and systems described herein to detect SNVs in the nucleic acid molecule of a subject, generate one or more metrics (or CPAS), the likelihood that cancer in a subject will progress or recur can be determined. Thus, the methods described herein can also be used to facilitate the prescribing of a management program or treatment regimen for a subject. For example, if it is determined that the subject's cancer is likely to progress or recur, then treatment of the subject with an appropriate therapy (e.g. a different and/or more aggressive therapy) can be initiated, or a current therapy can be maintained. Alternatively, if it is determined that the subject's cancer is unlikely to progress or recur, then treatment of the subject may be stopped, reduced or maintained.

As demonstrated in the examples below, subjects with a cancer that is likely to progress or recur have a different profile of metrics (or CPAS) compared to subject's with a cancer that is unlikely to progress or recur. A profile of metrics for a subject, i.e. a sample profile, can therefore be generated and compared to a reference profile of metrics so as to determine whether the subject has a cancer that is likely or unlikely to progress or recur. Profiles of the present disclosure reflect an evaluation of at least any 1, 2, 3, 4, 5, 6, 7, 8, 9, 10, 20, 30, 40 or more metrics (or CPAS) as described above. Reference profiles may correlate with, or be representative of, a subject that has a cancer that is likely to progress or recur, and/or may correlate with, or be representative of, a subject that has a cancer that is unlikely to progress or recur. When a comparison between the sample profile and the reference profile is made, similarities or differences in the profiles can indicate that the subject has a cancer that is likely, or unlikely, to recur or progress. For example, where a reference profile correlates with, or is representative of, a subject that has a cancer that is likely to progress or recur (e.g. as expressed by a particular PFS time, such as a relatively low PFS time), and the sample profile is similar to or essentially the same as that reference profile, then a determination can be made that the subject from which the sample profile was derived has a cancer that is likely to progress or recur. Conversely, where a reference profile correlates with, or is representative of, a subject that has a cancer that is unlikely to progress or recur (e.g. as expressed by a particular PFS time, such as a relatively high PFS time), and the sample profile is similar to or essentially the same as that reference profile, then a determination can be made that the subject from which the sample profile was derived has a cancer that is unlikely to progress or recur. As would be appreciated, the set of metrics in a profile that can distinguish a cancer that progresses compared to one that doesn't may be different for different types of cancer. For example, the set of metrics in a profile that can distinguish breast cancer that is likely to progress from breast cancer that is unlikely to progress may be different to the set of metrics in a profile that can distinguish skin cancer that is likely to progress from skin cancer that is unlikely to progress. While there may be some metrics that are overlapping (i.e. used to generate both the breast cancer and skin cancer profiles), some metrics may be used in only one of the profiles. Consequently, the reference profile generated and/or utilized in the methods of the present disclosure will typically be specific for a particular type of a cancer, which will be the same type of cancer as that of the subject being assessed, i.e. where the subject being assessed has a breast cancer, the reference profile will correlate with, or be representative of, a subject that has a breast cancer that is unlikely to, or is likely to, progress or recur.

Reference profiles are determined based on data obtained in the evaluation of reference metrics or CPAS in individuals that have a known phenotype, disease state or risk of developing a disease. Thus, for example, the reference profiles can be based on the data obtained in the evaluation of metrics in individuals that have or had cancers that did not progress or recur. In such instances, the reference profile correlates to, or is representative of, a subject that has a cancer is unlikely to progress or recur. In other examples, the reference profile is based on the data obtained in the evaluation of metrics in individuals that have or had a cancer that progressed or recurred. In such instances, the reference profile correlates to, or is representative of, a subject that has a cancer that is unlikely progress or recur. The individuals used to generate the reference profile may be age, gender and/or ethnicity matched, or not. As would be appreciated, the type of cancer will typically be matched, i.e. the reference profile will be determined based on data obtained from a reference or control subject with the same type of cancer as that of the subject being assessed using the methods of the disclosure.

In particular embodiments, reference profiles are produced using, and encompass, computational models, such as those formed using various analytical techniques such as machine learning techniques. Computational models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference. Non-limiting examples of techniques that can be used to produce classification models include deep learning techniques such as Deep Boltzmann Machine, Deep Belief Networks, Convolutional Neural Networks, Stacked Auto Encoders; ensemble techniques such as Random Forest, Gradient Boosting Machines, Boosting, Bootstrapped Aggregation, Ada Boost, Stacked Generalization, Gradient Boosted Regression Trees; neural network techniques such as Radial Basis Function Network, Perceptron, Back-Propagation, Hopfield Network; regularization methods such as Ridge Regression, Least Absolute Shrinkage and Selection Operator, Elastic Net, Least Angle Regression; regression methods such as Linear Regression, Ordinary Least Squares Regression, Multiple Regression, Probit Regression, Stepwise Regression, Multivariate Adaptive Regression Splines, Locally Estimated Scatterplot Smoothing, Logistic Regression, Support Vector Machines, Poisson Regression, Negative Binomial Regression, Multinomial Logistic Regression; Bayesian techniques such as Naïve Bayes, Average One-Dependence Estimators, Gaussian Naive Bayes, Multinomial Naive Bayes, Bayesian Belief Network, Bayesian Network; decision trees such as Classification and Regression Tree, Iterative Dichotomiser, C4.5, C5.0, Chi-squared Automatic Interaction Detection, Decision Stump, Conditional Decision Trees, M5; dimensionality reduction such as Principle Component Analysis, Partial Least Squares Regression, Sammon Mapping, Multidimensional Scaling, Projection Pursuit, Principle Component Regression, Partial Least Squares Discriminant Analysis, Mixture Discriminant Analysis, Quadratic Discriminant Analysis, Regularized Discriminant Analysis, Flexible Discriminant Analysis, Linear Discriminant Analysis, t-Distributed Stochastic Neighbour Embedding; instance-based techniques such as K-Nearest Neighbour, Learning Vector Quantization, Self-Organizing Map, Locally Weighted Learning; clustering methods such as k-Means, k-Modes, k-Medians, DBSCAN, Expectations Maximization, Hierarchical Clustering; adaptations, extensions, and combinations of the previously mentioned approaches.

Data from individuals who are known to have a cancer that has not progressed or recurred, and/or data from individuals who are known to have a cancer that has progressed or recurred, can be used to train a computational model. Such data is typically referred to as a training data set. Once trained, the computational model can recognize patterns in data generated using unknown samples, e.g. the data from patients with cancer used to generate the sample profiles. The sample profile can then be applied to the computational model to classify the sample profile into classes, e.g. having a cancer that is likely to progress or recur, or is unlikely to progress or recur.

In some embodiments, reference profiles are generated based on predetermined range intervals or cut-offs for each metric assessed. For example, a reference score is attributed to each metric that is outside a predetermined range interval or is above or below a predetermined cut-off, and the total reference score is then calculated by combining all of the scores. This total reference score is then used to generate a predetermined threshold score, above or below which represents a particular known phenotype, disease state or risk of developing a disease, e.g. below the threshold represents a subject whose cancer is unlikely to recur or progress, and above the threshold represents a subject whose cancer is likely to recur or progress. The threshold score therefore represents a score that differentiates those whose cancer is likely to progress or recur and from those whose cancer is unlikely to progress or recur, and can be readily established by those skilled in the art based on values and scores obtained using control subjects (e.g. control subjects known to have or have had a cancer that progresses or recurs, and/or control subjects known to have or to have had a cancer that does not progress or recur). The score for each metric may be the same or may be different (e.g. may be “weighted” such that one metric that is outside a predetermined range interval or above or below a cut-off might be given a score that is more or less than another metric). In a particular example, each metric that is outside a predetermined range interval or is above or below a cut-off is given a score of 1.

The predetermined range interval, or cut-off, for a metric can be determined by assessing a metric in two or more subjects known to have or have had a cancer that progresses or recurs, and/or two or more subjects known to have or to have had a cancer that does not progress or recur. A range interval for the metric is then calculated to set the upper and lower limits of what would be considered target values for that metric. A cut-off for the metric can be similarly calculated to set the upper or lower limit of what would be considered target values for that metric. In some examples, the range interval is calculated by measuring the average value of the metric plus or minus n standard deviations, whereby the lower limit of the range interval is the average minus n standard deviations and the upper limit of the range interval is the average plus n standard deviations. Cut-off can be similarly calculated. In such examples, n can be 1 or more than or less than 1, e.g. 0.1, 0.2, 0.3, 0.4, 0.5, 0.6, 0.7, 0.8, 0.9, 1, 1.5, 2, 2.5, etc. In still further examples, the upper and lower limits of the predetermined range interval or cut-off are established using receiver operating characteristic (ROC) curves. The subjects used to determine the predetermined range interval or cut-off can be of any age, sex or background, or may be of a particular age, sex, ethnic background or other subpopulation. Thus, in some embodiments, two or more predetermined normal range intervals or cut-offs can be calculated for the same metric, whereby each range interval or cut-off is specific for a particular subpopulation, e.g. a particular sex, age group, ethnic background and/or other subpopulation. The predetermined range interval or cut-off can be determined using any technique known to those skilled in the art, including manual methods of calculation, an algorithm, a neural network, a support vector machine, deep learning, logistic regression with linear models, machine learning, artificial intelligence and/or a Bayesian network.

In some examples, the reference and sample profiles include a plurality of metrics that comprises 5 or more metrics selected from the metrics set forth in Table D and metrics related to the metrics set forth in Table D. In particular examples, the profiles include a plurality of metrics that comprises least or about 10, 15, 20, 35, 30, 40, 45, 50, 55, 60, 65, 70, 75, 80, 85, 90, 95, 100 or more metrics selected from the metrics set forth in Table D and metrics related to the metrics set forth in Table D.

In some examples, such as where progression or recurrence of mesothelioma is being assessed, the profiles include a plurality of metrics that comprises least or about 2, 3, 4, 5, 6, 7, 8, 9, 10, 11, 12, 13, 14, 15, 16, 17, 18, 19, or all metrics selected from cds:3Gen2_C-C-C MC3%, g:A3Bj_RT-C-G C>T+G>A g cds:3Gen3_GG-C-non-syn cds:A3Gb_-C-G G>A at MC2 motif %, cds:A3B_T-C-W MC1 cds:3Gen1_-C-GC MC2%, cds:A3Gb_-C-G G>A MC2 Hits, cds:A1_-C-A G>A at MC3 cds cds:3Gen3_AG-C-MC2 cds:ADAR_W-A-non-syn cds:A3Bj_RT-C-G Ti g:3Gen2_T-C-G C>T+G>A g cds:AIDe_WR-C-GW Hits, cds:3Gen2_A-C-G MC2 non-syn cds:A3Gi_SG-C-G non-syn g:ADAR_W-A-A>G+T>C cds:2Gen1_-C-C G>T at MC1 cds:A3Gi_SG-C-G MC2%, cds:A3Bf_ST-C-G Ti cds:ADAR_3 Gen2_G-A-C non-syn %, and related metrics thereto. In further examples of where progression or recurrence of mesothelioma is being assessed, the profiles include a plurality of metrics that comprises least or about 2, 3, 4, 5, 6, 7, 8, 9, 10, 11, 12, 13, 14, 15, 16, 17, 18, 19, or all metrics selected from cds:A3Bf_ST-C-G Ti %; g:3Gen2_T-C-G C>T+G>A g %; cds:2Gen1_-C-C C>T at MC1%; cds:All C Ti/Tv; g:3Gen3_CA-C-C>T+G>A g %; cds:3Gen2_C-C-C MC3%; cds:A3Gn_YYC-C-S C>T %; cds:A3G_C-C-MC3%; cds:3Gen3_GG-C-non-syn %; g:3Gen2_A-C-C C>A+G>T g %; cds:4Gen3_TT-C-C %; cds:3Gen2_C-C-T MC3%; g:2Gen1_-C-T C>G+G>C g %; cds:Primary Deaminase %; cds:A3Gb_-C-G G>A at MC2 motif %; cds:4Gen3_CA-C-C %; cds:A3G_C-C-G>T %; cds:A3Gi_SG-C-G non-syn %; g:C>G+G>C %; cds:Other MC3%; cds:A3B_T-C-W G>A motif %, and related metrics thereto.

In some examples, such as where progression or recurrence of adrenocortical carcinoma is being assessed, the profiles include a plurality of metrics that comprises least or about 2, 3, 4, 5, 6, 7, 8, 9, 10, 11, 12, 13, 14, 15, 16, 17, 18, 19, or all metrics selected from g:A3F_T-C-Hits, cds:3Gen1_-C-TG G non-syn cds:3Gen2_C-C-T MC3%, cds:All G total, g:3Gen1_-C-TC C>T+G>A g cds:3Gen3_CT-C-MC3%, cds:All G nc:A3G_C-C-C>T+G>A nc cds:A3B_T-C-W G>A motif %, cds:AIDc_WR-C-GS cds:3Gen1_-C-GT G>A motif %, cds:A3B_T-C-W MC3 non-syn cds:3Gen3_TG-C-G>A cds:ADAR_2 Gen2_G-T-MC2%, cds:3Gen3_TG-C-G Ti/Tv %, cds:4Gen3_TT-C-C cds:2Gen1_-C-C C>A cds:A3G_C-C-C>T at MC1 cds:AIDb_WR-C-G G non-syn cds:A3G_C-C-MC3%, and related metrics thereto. In further examples of where progression or recurrence of adrenocortical carcinoma is being assessed, the profiles include a plurality of metrics that comprises least or about 2, 3, 4, 5, 6, 7, 8, 9, 10, 11, 12, 13, 14, 15, 16, 17, 18, 19, 20, 25, 30, 35 or all metrics selected from cds:All G total; cds:3Gen1_-C-TG G non-syn %; g:A3F_T-C-Hits; cds:3Gen3_GG-C-non-syn %; cds:3Gen1_-C-GT G>A motif %; cds:A3Bj_RT-C-G Ti %; cds:3Gen2_C-C-T MC3%; nc:A3G_C-C-C>T+G>A nc %; cds:AIDd_WR-C-Y %; cds:3Gen1_-C-TC C>T cds %; cds:A3B_T-C-W G>A motif %; g:CG total; cds:A3G_C-C-MC3%; cds:AIDb_WR-C-G G non-syn %; cds:A3G_C-C-C>T at MC1%; cds:3Gen3_TG-C-G>A %; g:3Gen3_GA-C-C>A+G>T g %; cds:3Gen2_A-C-G MC2 non-syn %; cds:3Gen3_CT-C-MC3%; cds:ADAR_2 Gen2_G-T-MC2%; cds:ADAR_3 Gen3_CA-A-Ti %; g:AIDh_WR-C-T C>A+G>T g %; cds:A3B_T-C-W MC3 non-syn %; cds:2Gen1_-C-C C>A %; cds:A1_-C-A G>A at MC3 cds %; cds:3Gen1_-C-CA Ti C:G %; cds:ADAR_W-A-non-syn %; cds:3Gen1_-C-CA Ti %; cds:All G %; g:3Gen2_T-C-G C>T+G>A g %; cds:A3Gb_-C-G MC1%; cds:A3B_T-C-W G non-syn %; nc:2Gen2_A-C-C>T+G>A nc %; cds:A3Gi_SG-C-G non-syn %; cds:Other G MC3 Ti/Tv %; cds:A3Gb_-C-G G>A at MC2 motif %; cds:A3B_T-C-W Ti %; and g:2Gen1_-C-T %, and related metrics thereto.

In other examples, such as where progression or recurrence of brain tumour (e.g. lower grade glioma) is being assessed, the profiles include a plurality of metrics that comprises least or about 2, 3, 4, 5, 6, 7, 8, 9, 10, 11, 12, 13, 14, 15, 16, 17, 18, 19, or all metrics selected from g:CG total, cds:AIDc_WR-C-GS MC3, cds:A3B_T-C-W G non-syn cds:AIDd_WR-C-Y g:AIDc_WR-C-GS Hits, cds:3Gen2_A-C-C non-syn g:3Gen3_GA-C-C>A+G>T g cds:2Gen2_G-C-Hits, cds:4Gen3_TA-C-C non-syn, nc:2Gen2_A-C-C>T+G>A nc cds:Other MC3 C cds:3Gen2_T-C-G Ti/Tv g:3Gen2_A-C-C C>A+G>T g, g:3Gen3_CA-C-C>T+G>A g cds:3Gen2_T-C-C MC1 g:ADAR_2 Gen1_-T-T A>T+T>A g:ADAR_2 Gen2_G-T-A>T+T>A g:2Gen1_-C-T cds:ADAR_3 Gen1_-A-CA cds:ADAR_2 Gen2_T-T-%, and related metrics thereto. In other examples of where progression or recurrence of brain tumour (e.g. lower grade glioma) is being assessed, the profiles include a plurality of metrics that comprises least or about 2, 3, 4, 5, 6, 7, 8, 9, 10, 11, 12, 13, 14, 15, 16, 17, 18, 19, 20, 30, 40, 50, 60, 70, 80 or all metrics selected from g:CG total; cds:AIDd_WR-C-Y %; variants in VCF; cds:4Gen3_TA-C-C non-syn %; cds:3Gen2_C-C-T MC3%; cds:AIDd_WR-C-Y G>C %; cds:A3Gb_-C-G MC1%; g:3Gen2_T-C-G C>T+G>A g %; cds:A3B_T-C-W G non-syn %; g:3Gen3_GA-C-C>A+G>T g %; cds:2Gen2_G-C-Hits; cds:AIDc_WR-C-GS MC3%; cds:All G total; cds:All A non-syn %; cds:ADAR_2 Gen2_T-T-%; cds:3Gen2_A-C-C non-syn %; g:3Gen3_CA-C-C>T+G>A g %; g:ADARk_CW-A-A>G+T>C g %; nc:ADARb_W-A-Y A>G+T>C nc %; g:2Gen1_-C-T %; cds:Other MC3 C %; g:2Gen1_-C-T C>G+G>C g %; cds:ADAR_W-A-non-syn %; g:3Gen2_A-C-C C>A+G>T g %; g:ADAR_2 Gen2_G-T-A>T+T>A %; cds:A3G_C-C-C>T at MC1%; cds:3Gen1_-C-GC MC2%; cds:3Gen2_G-C-T %; cds:A3F_T-C-G>C %; g:4Gen3_GG-C-G C>T+G>A g %; cds:A3Gb_-C-G G>A at MC2 motif %; cds:ADARb_W-A-Y MC2%; cds:All G %; g:A3F_T-C-Hits; cds:3Gen2_T-C-C MC1%; cds:A3B_T-C-W Ti %; cds:ADAR_3 Gen1_-A-AT Ti %; cds:ADARh_W-A-S T>C %; cds:A3Gn_YYC-C-S C>T %; cds:A3Ge_SC-C-GS %; cds:2Gen2_A-C-MC3%; cds:ADAR_2 Gen2_G-T-MC2%; cds:ADAR_3 Gen3_CA-A-Ti %; cds:Primary Deaminase %; g:C>G+G>C %; cds:A3Bf_ST-C-G Ti %; cds:3Gen3_CT-C-MC3%; cds:A3Gi_SG-C-G non-syn %; cds:Other MC3%; cds:ADAR_3 Gen1_-A-CA %; cds:A3F_T-C-C>A %; cds:2Gen1_-C-C C>T at MC1%; cds:A3Gc_C-C-GW C>T motif %; cds:AIDc_WR-C-GS %; g:ADAR_2 Gen1_-T-T A>T+T>A %; cds:A3B_T-C-W MC1%; cds:ADAR_3 Gen2_G-A-C non-syn %; cds:2Gen1_-C-C C>A %; cds:3Gen1_-C-GT G>A motif %; cds:A3Bj_RT-C-G Ti %; g:3Gen1_-C-TC C>T+G>A g %; g:C>A+G>T %; cds:3Gen2_A-C-C MC2%; cds:2Gen1_-C-C MC2%; g:3Gen2_G-C-T %; g:A3Bj_RT-C-G C>T+G>A g %; g:ADAR_W-A-A>G+T>C %; cds:3Gen3_AT-C-C:G %; cds:3Gen1_-C-TG G non-syn %; cds:Other G MC3 Ti/Tv %; cds:A3Gb_-C-G G>A MC2 Hits; cds:3Gen1_-C-TC C>T cds %; cds:2Gen1_-C-T MC3 non-syn %; cds:AIDb_WR-C-G G non-syn %; g:AIDc_WR-C-GS Hits; cds:3Gen2_T-C-C MC3%; cds:3Gen2_T-C-G Ti/Tv %; cds:A1_-C-A G>A at MC3 cds %; nc:A3G_C-C-C>T+G>A nc %; nc:2Gen2_A-C-C>T+G>A nc %; cds:3Gen3_TG-C-G Ti/Tv %; cds:3Gen1_-C-CA Ti %; cds:3Gen3_TG-C-G>A %; cds:3Gen3_CT-C-G non-syn %; cds:All C Ti/Tv %; cds:A3G_C-C-MC3%; cds:ADARc_SW-A-Y MC2%; and cds:3Gen3_GG-C-non-syn and related metrics thereto.

In further examples, such as where progression or recurrence of sarcoma is being assessed, the profiles include a plurality of metrics that comprises least or about 2, 3, 4, 5, 6, 7, 8, 9, 10, 11, 12, 13, 14, 15, 16, 17, 18, 19, or all metrics selected from nc:ADARb_W-A-Y A>G+T>C nc g:ADARk_CW-A-A>G+T>C g cds:ADAR_3 Gen3_CA-A-Ti cds:A3G_C-C-G>T cds:4Gen3_TT-C-T cds:ADARc_SW-A-Y T>C cds nc:ADARc_SW-A-Y A>G+T>C nc cds:A3F_T-C-G>C g:C>A+G>T cds:2Gen1_-C-T MC3 non-syn nc:ADARb_W-A-Y cds:AIDd_WR-C-Y C>A cds cds:Primary Deaminase cds:4Gen3_CA-C-C MC1 g:C>G+G>C, g:2Gen1_-C-T C>G+G>C g g:AIDh_WR-C-T C>A+G>T g cds:A3Ge_SC-C-GS cds:ADAR_3 Gen3_CT-A-A>G motif %, cds:ADARf_SW-A-MC2%, and related metrics thereto. In further examples of where progression or recurrence of sarcoma is being assessed, the profiles include a plurality of metrics that comprises least or about 2, 3, 4, 5, 6, 7, 8, 9, 10, 11, 12, 13, 14, 15, 16, 17, 18, 19, 20, 25, 30 or all metrics selected from cds:Other MC3 C %; nc:ADARb_W-A-Y A>G+T>C nc %; cds:4Gen3_TT-C-T; g:ADARk_CW-A-A>G+T>C g %; g:ADARn_-A-WA A>G+T>C %; cds:A3G_C-C-G>T %; cds:A3Gb_-C-G MC1%; nc:ADARb_W-A-Y %; cds:A3Ge_SC-C-GS %; cds:Primary Deaminase %; cds:ADAR_2 Gen2_G-T-MC2%; g:4Gen3_GG-C-G C>T+G>A g %; cds:2Gen1_-C-C MC2%; cds:3Gen1_-C-GT G>A motif %; cds:A3Gn_YYC-C-S C>T %; cds:2Gen1_-C-C C>T at MC1%; cds:A3B_T-C-W MC3 non-syn %; cds:AIDd_WR-C-Y %; g:3Gen3_CA-C-C>T+G>A g %; cds:All A non-syn %; g:2Gen1_-C-T C>G+G>C g %; cds:ADARb_W-A-Y MC2%; cds:All G %; g:A3Bj_RT-C-G C>T+G>A g %; cds:A3Gn_YYC-C-S C>T at MC3 cds %; cds:A3B_T-C-W G non-syn %; cds:A3G_C-C-MC3%; cds:All G total; cds:CDS Variants; g:CG total; g:3Gen2_T-C-G C>T+G>A g %; cds:A3B_T-C-W MC1%; cds:ADAR_3 Gen3_CA-A-Ti %; cds:AIDc_WR-C-GS %, and related metrics thereto.

In other examples, such as where progression or recurrence of lung cancer (e.g. lung squamous cell carcinoma) is being assessed, the profiles include a plurality of metrics that comprises least or about 2, 3, 4, 5, 6, 7, 8, 9, 10, 11, 12, 13, 14, 15, 16, 17, 18, 19, or all metrics selected from cds:ADARp_-A-WT A>G at MC2 cds cds:3Gen1_-C-TC C>T cds cds:AIDd_WR—C-Y G>C cds:ADAR_3 Gen3_AC-A-A>G cds cds:3Gen1_-C-CT C>T at MC2 cds cds:A3Go_TC-C-G MC1 non-syn cds:3Gen2_G-C-T C>A motif %, nc:2Gen1_-C-T C>A+G>T nc cds:ADAR_2 Gen2_A-T-A>C at MC1 motif %, cds:4Gen3_CA-C-C %, cds:A3Gn_YYC-C-S C>T at MC3 cds %, cds:3Gen1_-C-AG G Ti/Tv cds:ADARh_W-A-S T>C cds:3Gen1_-C-CC C>T at MC1 motif %, cds:2Gen1_-C-C C>T at MC1 g:ADAR_4 Gen3_AG-A-G A>C+T>G cds:4Gen3_CT-C-C C>T at MC1 cds:ADAR_3 Gen1_-A-CC A>G cds cds:A3Gn_YYC-C-S C>T cds:ADAR_W-A-T>C at MC2%, and related metrics thereto. In further examples of where progression or recurrence of lung cancer (e.g. lung squamous cell carcinoma) is being assessed, the profiles include a plurality of metrics that comprises least or about 2, 3, 4, 5, 6, 7, 8, 9, 10, 11, 12, 13, 14, 15, 16, 17, 18, 19, 20, 30, 40, 50, 60, 70, 80, 90 or all metrics selected from cds:3Gen1_-C-CC C>T at MC1 motif %; cds:3Gen1_-C-CT C>T at MC2 cds %; cds:ADARp_-A-WT A>G at MC2 cds %; cds:Other MC3 C %; cds:Other MC3%; cds:A3Gb_-C-G MC1%; g:3Gen1_-C-TC C>T+G>A g %; cds:ADAR_W-A-A>G at MC3%; cds:ADAR_W-A-non-syn %; cds:ADAR_3 Gen3_AC-A-A>G cds %; cds:2Gen1_-C-C C>A %; cds:ADARf_SW-A-MC2%; g:ADAR_2 Gen2_G-T-A>T+T>A %; cds:4Gen3_GC-C-A %; cds:A3Go_TC-C-G MC1 non-syn %; g:3Gen2_G-C-T %; cds:A3G_C-C-C>T at MC1%; cds:AIDc_WR-C-GS MC3%; cds:3Gen1_-C-GT G>A motif %; nc:2Gen1_-C-T C>A+G>T nc %; cds:ADARc_SW-A-Y MC2%; cds:ADARh_W-A-S T>C %; cds:2Gen1_-C-C C>T at MC1%; g:ADAR_2 Gen1_-T-T A>T+T>A %; cds:AIDd_WR-C-Y C>A cds %; nc:A3G_C-C-C>T+G>A nc %; cds:A3Gc_C-C-GW C>T motif %; cds:ADAR_3 Gen1_-A-AT Ti %; cds:3Gen3_CT-C-MC3%; cds:4Gen3_CT-C-C C>T at MC1%; cds:3Gen2_T-C-C MC1%; cds:A3G_C-C-G>T %; cds:3Gen1_-C-CA Ti %; cds:3Gen1_-C-TG G non-syn %; cds:3Gen2_A-C-C non-syn %; g:2Gen1_-C-T C>G+G>C g %; cds:All A non-syn %; cds:A3Gi_SG-C-G MC2%; cds:Primary Deaminase %; cds:4Gen3_TT-C-T %; g:A3Bj_RT-C-G C>T+G>A g %; cds:3Gen2_T-C-C MC3%; cds:4Gen3_TT-C-C %; cds:3Gen1_-C-CA Ti C:G %; cds:A1_-C-A G>A at MC3 cds %; cds:A3Gb_-C-G G>A at MC2 motif %; cds:3Gen3_CT-C-G non-syn %; cds:3Gen2_G-C-T C:G %; cds:A3Ge_SC-C-GS %; cds:3Gen3_TG-C-G>A %; g:C>A+G>T %; cds:4Gen3_CA-C-C %; cds:AIDd_WR-C-Y G>C %; cds:All G %; cds:3Gen3_TT-C-C>A at MC1 motif %; g:AIDh_WR-C-T C>A+G>T g %; g:4Gen3_GG-C-G C>T+G>A g %; cds:3Gen2_G-C-T C>A motif %; nc:ADARc_SW-A-Y A>G+T>C nc %; g:3Gen2_A-C-C C>A+G>T g %; cds:A3B_T-C-W Ti %; g:3Gen3_GA-C-C>A+G>T g %; cds:3Gen3_CT-C-C>T at MC1 motif %; cds:ADAR_3 Gen1_-A-CC A>G cds %; cds:3Gen1_-C-TC C>T cds %; cds:4Gen3_CA-C-C MC1%; cds:3Gen2_G-C-T %; nc:2Gen2_A-C-C>T+G>A nc %; cds:3Gen2_A-C-C MC2%; cds:A3F_T-C-C>A %; cds:CDS Variants; cds:ADAR_3 Gen3_CA-A-Ti %; cds:3Gen3_GG-C-non-syn %; cds:ADARb_W-A-Y MC2% %; g:ADAR_W-A-A>G+T>C %; cds:3Gen3_AT-C-C:G %; cds:2Gen1_-C-C G>T at MC1%; cds:A3G_C-C-MC3%; cds:3Gen2_C-C-C MC3%; cds:A3B_T-C-W G>A motif %; cds:A3F_T-C-G>C %; cds:ADAR_2 Gen2_G-T-MC2%; cds:3Gen1_-C-AG G Ti/Tv %; cds:A3Bj_RT-C-G Ti %; nc:ADARb_W-A-Y A>G+T>C nc %; cds:ADAR_2 Gen2_T-T-%; g:2Gen1_-C-T %; cds:4Gen3_AC-C-T Ti/Tv %; cds:A3Gi_SG-C-G non-syn %; cds:A3Bf_ST-C-G Ti %; g:ADARk_CW-A-A>G+T>C g %; cds:3Gen1_-C-GC MC2%; g:3Gen3_CA-C-C>T+G>A g %; cds:2Gen2_A-C-MC3%; variants in VCF; cds:4Gen3_AG-C-T MC1 non-syn %; g:3Gen2_T-C-G C>T+G>A g %; cds:A3Gn_YYC-C-S C>T at MC3 cds %; cds:ADAR_3 Gen1_-A-CA %; cds:4Gen3_TA-C-C non-syn %; cds:All C Ti/Tv %; cds:ADARc_SW-A-Y, and related metrics thereto.

In other examples, such as where progression or recurrence of skin cancer (e.g. melanoma) is being assessed, the profiles include a plurality of metrics that comprises least or about 2, 3, 4, 5, 6, 7, 8, 9, 10, 11, 12, 13, 14, 15, 16, 17, 18, 19, or all metrics selected from cds:4Gen3_AG-C-T MC1 non-syn cds:All A non-syn cds:3Gen1_-C-CG G>A at MC3%, cds:3Gen3_TT-C-C>A at MC1 motif %, cds:A3Gc_C-C-GW C>T motif %, cds:ADAR_W-A-A>G at MC3%, cds:ADARp_-A-WT T>A motif %, cds:3Gen3_CT-C-G non-syn cds:3Gen2_T-C-T G>A at MC2%, cds:ADAR_3 Gen1_-A-AT Ti cds:All C Ti/Tv cds:3Gen1_-C-TC C>T at MC3%, cds:4Gen3_AG-C-T G>A at MC1 motif %, cds:3Gen1_-C-CA Ti C:G cds:3Gen3_AT-C-G>A at MC2%, cds:4Gen3_TG-C-T Ti C:G cds:ADAR_3 Gen2_C-A-C T>G at MC3 cds cds:4Gen3_GC-C-C C>T at MC2%, cds:4Gen3_AC-C-T Ti/Tv cds:AIDh_WR-C-T G>A at MC2 cds %, and related metrics thereto. In further examples of where progression or recurrence of skin cancer (e.g. melanoma) is being assessed, the profiles include a plurality of metrics that comprises least or about 2, 3, 4, 5, 6, 7, 8, 9, 10, 11, 12, 13, 14, 15, 16, 17, 18, 19, 20, 30, 40, 50, 60, 70, 80, 90 or all metrics selected from cds:4Gen3_AG-C-T MC1 non-syn %; cds:3Gen1_-C-CG G>A at MC3%; cds:4Gen3_AC-C-T Ti/Tv %; g:C>G+G>C %; cds:A3B_T-C-W MC3 non-syn %; cds:All A non-syn %; cds:3Gen3_AG-C-MC2%; cds:A3B_T-C-W MC1%; cds:ADAR_3 Gen2_C-A-C T>G at MC3 cds %; cds:3Gen1_-C-TC C>T at MC3%; cds:4Gen3_GC-C-C C>T at MC2%; cds:All C Ti/Tv %; cds:A3Bj_RT-C-G Ti %; cds:AIDh_WR-C-T G>A at MC2 cds %; cds:4Gen3_TT-C-C %; cds:3Gen1_-C-CC C>T at MC1 motif %; cds:ADAR_2 Gen2_T-T-; cds:3Gen2_T-C-C MC1%; cds:All G %; cds:ADAR_W-A-A>G at MC3%; cds:A3G_C-C-MC3%; cds:Other MC3 C %; g:3Gen2_A-C-C C>A+G>T g %; cds:ADARc_SW-A-Y MC2%; cds:3Gen1_-C-CA Ti C:G %; cds:3Gen1_-C-TC C>T cds %; cds:3Gen2_C-C-C MC3%; cds:3Gen3_CT-C-C>T at MC1 motif %; g:ADAR_4 Gen3_AG-A-G A>C+T>G %; cds:3Gen3_CT-C-G non-syn %; cds:3Gen2_A-C-C non-syn %; cds:2Gen2_A-C-MC3%; cds:3Gen2_A-C-C MC2%; g:3Gen1_-C-TC C>T+G>A g %; cds:3Gen2_T-C-T G>A at MC2%; cds:2Gen1_-C-C C>T at MC1%; cds:AIDb_WR-C-G G non-syn %; cds:A3Gb_-C-G MC1%; cds:2Gen1_-C-C C>A %; cds:A3Ge_SC-C-GS %; g:ADARn_-A-WA A>G+T>C %; g:ADAR_W-A-A>G+T>C %; g:ADAR_2 Gen2_G-T-A>T+T>A %; g:AIDh_WR-C-T C>A+G>T g %; cds:4Gen3_TG-C-T Ti C:G %; cds:3Gen2_G-C-T C:G %; cds:3Gen2_T-C-C MC3%; nc:ADARb_W-A-Y %; cds:ADAR_3 Gen2_G-A-C non-syn %; cds:ADAR_3 Gen1_-A-AT Ti %; g:ADARk_CW-A-A>G+T>C g %; cds:3Gen1_-C-GC MC2%; cds:4Gen3_TA-C-C non-syn %; g:3Gen3_CA-C-C>T+G>A g %; cds:3Gen1_-C-AG G Ti/Tv %; cds:AIDc_WR-C-GS %; cds:A3Gn_YYC-C-S C>T at MC3 cds %; cds:2Gen1_-C-C MC2%; cds:3Gen3_GG-C-non-syn %; g:2Gen1_-C-T C>G+G>C g %; cds:A1_-C-A G>A at MC3 cds %; cds:A3G_C-C-C>T at MC1%; nc:ADARc_SW-A-Y A>G+T>C nc %; cds:ADAR_W-A-T>C at MC2%; cds:A3Go_TC-C-G MC1 non-syn %; cds:3Gen3_AT-C-C:G %; cds:ADARh_W-A-S T>C %; cds:A3G_C-C-G>T %; cds:ADARf_SW-A-MC2%; cds:ADAR_W-A-non-syn %; cds:ADARp_-A-WT T>A motif %; cds:4Gen3_AG-C-T G>A at MC1 motif %; cds:ADAR_3 Gen1_-A-CA %; cds:3Gen2_C-C-T MC3%; cds:3Gen1_-C-CT C>T at MC2 cds %; cds:A3B_T-C-W Ti %; g:2Gen1_-C-T %; cds:AIDc_WR-C-GS MC3%; cds:AIDe_WR-C-GW Hits; cds:AIDd_WR-C-Y C>A cds %; cds:ADARb_W-A-Y MC2%; cds:A3Gc_C-C-GW C>T motif %; cds:2Gen1_-C-C G>T at MC1%; cds:3Gen1_-C-CA Ti %; cds:Other G MC3 Ti/Tv %; cds:CDS Variants; cds:ADAR_3 Gen1_-A-CC A>G cds %; cds:A3Gn_YYC-C-S C>T %; cds:A3Bf_ST-C-G Ti %; cds:2Gen2_G-C-Hits; cds:AIDd_WR-C-Y %; cds:A3F_T-C-G>C %; cds:4Gen3_CT-C-C C>T at MC1%; cds:AIDd_WR-C-Y G>C %; cds:A3Gi_SG-C-G MC2%; cds:Other MC3%; nc:2Gen1_-C-T C>A+G>T nc %; cds:3Gen2_G-C-T %; g:3Gen2_T-C-G C>T+G>A g %; cds:ADARc_SW-A-Y T>C cds %; and related metrics thereto.

The methods of the present invention also extend to therapeutic or preventative protocols. In instances where a cancer is determined to be unlikely to progress or recur, treatment protocols may be amended to reduce the intensity of the treatment, or to remove a subject from a treatment regimen completely. In instances where a cancer is determined to be likely to progress or recur, protocols designed to reduce that likelihood may be designed and applied to a subject. For example, an appropriate therapeutic protocol can be designed for the subject and administered. This may include, for example, radiotherapy, surgery, chemotherapy, hormone ablation therapy, pro-apoptosis therapy and/or immunotherapy. In some examples, further diagnostic tests may be performed to confirm the diagnosis prior to therapy.

Radiotherapies include radiation and waves that induce DNA damage for example, γ-irradiation, X-rays, UV irradiation, microwaves, electronic emissions, radioisotopes, and the like. Therapy may be achieved by irradiating the localized tumour site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half life of the isotope, the strength and type of radiation emitted, and the uptake by the neoplastic cells.

Non-limiting examples of radiotherapies include conformal external beam radiotherapy (50-100 Grey given as fractions over 4-8 weeks), either single shot or fractionated, high dose rate brachytherapy, permanent interstitial brachytherapy, systemic radio-isotopes (e.g., Strontium 89). In some embodiments the radiotherapy may be administered in combination with a radiosensitizing agent. Illustrative examples of radiosensitizing agents include but are not limited to efaproxiral, etanidazole, fluosol, misonidazole, nimorazole, temoporfin and tirapazamine.

Chemotherapeutic agents may be selected from any one or more of the following categories:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyridines like 5-fluorouracil and tegafur, raltitrexed, methotrexate, cytosine arabinoside and hydroxyurea; anti-tumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and docetaxel; and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazoli-n-4-amine (CI 1033)), for example inhibitors of the platelet-derived growth factor family and for example inhibitors of the hepatocyte growth factor family;

(v) anti-angiogenic agents such as those which inhibit the effects of vascular endothelial growth factor, (for example the anti-vascular endothelial cell growth factor antibody bevacizumab [AVASTIN™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example those which are directed to the targets listed above, such as ISIS 2503, an anti-ras antisense; and

(viii) gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Immunotherapy approaches, include for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

Examples of other cancer therapies include phototherapy, cryotherapy, toxin therapy or pro-apoptosis therapy. One of skill in the art would know that this list is not exhaustive of the types of treatment modalities available for cancer and other hyperplastic lesions.

In some instances, where a metric is indicative of the activity of a deaminase, therapy or preventative measures may include administration to the subject of an inhibitor of that deaminase. Inhibitors can include, for example, siRNAs, miRNAs, protein antagonists (e.g., dominant negative mutants of the mutagenic agent), small molecule inhibitors, antibodies and fragments thereof. For example, commercially available siRNAs and antibodies specific for APOBEC cytidine deaminases and AID are widely available and known to those skilled in the art. Other examples of APOBEC3G inhibitors include the small molecules described by Li et al. (ACS. Chem. Biol., (2012) 7(3): 506-517), many of which contain catechol moieties, which are known to be sulfhydryl reactive following oxidation to the orthoquinone. APOBEC1 inhibitors also include, but are not limited to, dominant negative mutant APOBEC1 polypeptides, such as the mu1 (H61K/C93S/C96S) mutant (Oka et al., (1997) J. Biol. Chem. 272: 1456-1460).

Typically, therapeutic agents will be administered in pharmaceutical compositions together with a pharmaceutically acceptable carrier and in an effective amount to achieve their intended purpose. The dose of active compounds administered to a subject should be sufficient to achieve a beneficial response in the subject over time such as a reduction in, or relief from, the symptoms of cancer, and/or the reduction, regression or elimination of tumours or cancer cells. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner, and those of skill in the art may readily determine suitable dosages of the therapeutic agents and suitable treatment regimens without undue experimentation.

The present invention can be practiced in the field of predictive medicine for the purposes of predicting the progression or recurrence of a cancer or tumour in a subject.

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavor to which this specification relates.

In order that the invention may be readily understood and put into practical effect, particular preferred embodiments will now be described by way of the following non-limiting examples.

### EXAMPLES

### Example 1

### Analysis of Patient Data

### A. Patient Data

The Cancer Genome Atlas (TCGA) is a collaboration between the National Cancer Institute (NCI) and the National Human Genome Research Institute (NHGRI). The goal of the TCGA is to conduct a comprehensive characterization of different cancer types in a large patient cohort to further our understanding of cancer aetiology. An expanding collection of landmark scientific findings have resulted from this collaboration (e.g. https://cancergenome.nih.gov/publications) and further analysis of this remarkable resource is ongoing. A prominent TCGA initiative is the ‘PanCancer Atlas’ project conducted by the Multi-Center Mutation-Calling in Multiple Cancers (MC3) network. The PanCancer Atlas is a reanalysis of 10,437 tumours from 33 of the most prevalent forms of cancer in the TCGA dataset.

TCGA PanCancer Atlas genomic data is stored and maintained by the NIH Genomic Data Commons (https://gdc.cancer.gov/access-data/data-access-processes-and-tools) and was accessed and visualized via the cBioPortal for Cancer Genomics (https://www.cbioportal.org/) (Cerami et al., 2012; Gao et al., 2013). Patients were recruited and biospecimens processed as described by Bailey et al. (2018). Cancer types included in the PanCancer Atlas include, for example, Adrenocortical Carcinoma (ADCC), Brain Lower Grade Glioma (BLGG), Lung Squamous Cell Carcinoma (LUSC), Mesothelioma (MESO), Pancreatic Adenocarcinoma (PAAD), Sarcoma (SARC), and Skin Cutaneous Melanoma (SKCM). Genomic data was obtained for all patients in the TCGA PanCancer Atlas.

Patients in the TCGA PanCancer Atlas cohort that had Progression Free Survival (PFS) documented were analyzed; those without a known PFS were excluded from analysis. For each cancer type, patients were categorized as “PFS_Low”: patients that progressed before the predetermined cancer-type-specific cut off; and “PFS_High”: patients that did not progress before the cut off. For each cancer type, groups were then compared using at least one computational model.

Metrics were determined as discussed below and computational models using various metrics were trained using ˜75% of the patient IIF profiles, hyperparameters were tuned using ˜10% of the profiles, and ‘blind’ predictions were made on ˜15% of profiles (sequestered before analysis). The overall accuracy, sensitivity and specificity were reported for the predictions made on patients excluded from training or tuning the model. IIF metrics contributing to the computational models were obtained, visualized, compared and validated. Concordant metrics were retained and used to evaluate the ‘blind’ patient predictions.

In these examples the models are an ensemble of weak prediction models (decision trees) with stochastic gradient descent used for optimisation. The “XGBoost” algorithm was used in these examples (Chen, T., & Guestrin, C. (2016). Xgboost: A scalable tree boosting system. In Proceedings of the 22nd acm sigkdd international conference on knowledge discovery and data mining (pp. 785-794). ACM).

The parameters used to train the XGBoost models were optimised using standard methods employing the ‘MLR’ software package (Bischl B, Lang M, Kotthoff L, Schiffner Richter J, Studerus E, Casalicchio G, Jones Z (2016). “mlr: Machine Learning in R.” Journal of Machine Learning Research, 17(170), 1-5. http://jmlr.org/papers/v17/15-066.html).

### B. Determining Metrics

Full genome sequences from patients were analyzed to identify single nucleotide variants (SNVs). Briefly, sequences were formatted in a .vcf file using the hg37 genome coordinates as a reference.

Each variant in the .vcf file was analyzed and selected for further consideration if it was a simple single nucleotide substitution and was not an insertion or deletion. The following steps were then performed in instances where SNVs in a motif and/or codon context was being assessed:

a) the codon context within the structure of the mutated codon (MC) was determined, i.e. the position of the SNV within the encoding triplet was determined, wherein the first position (read from 5′ to 3′) is referred to as MC1 (or MC-1 site), the second position is referred to as MC2 (or MC-2 site) and the third position is referred to as MC3 (or MC-3 site);

b) a nine-base window was extracted from the surrounding genome sequence such that the sequence of three complete codons was obtained. The direction of the gene was used for determining 5′ and 3′ directions, and for determining the correct strand of the nine bases. The nine-base window was always reported according to the direction of the gene such that bases in the window around variants in genes on the reverse strand of the genome are reverse complimented in relation to the genome, but in the forward direction in relation to the gene. By convention, this context is always reported in the same strand of the gene. Positive strand genes will have codon context bases from the positive strand of the reference genome, and negative strand genes will have codon context bases from the negative strand of the reference genome; and/or

c) motif searching was performed using motifs, such as described in Tables B and C to determine whether the variation was within such a motif.

### C. Metric Definitions

1. Regions

To enable analysis all SNVs are classified as coding (cds) or non-coding (nc), where cds SNVs are those within nucleic acid that encodes an amino acid in any known protein isoform, and nc SNVs are present in any other region of the genome that is not protein coding. This may be 5′ or 3′ UTRs, intronic region, intergenic region, non-coding RNA region or any other non-coding region. “Genomic region (g)” includes all SNVs, i.e. coding and non-coding SNVs.

2. Motif Metrics

All motifs were analysed in pairs, the forward motif and the equivalent reverse complement motif. Searching for the reverse complement motif is equivalent to searching for the forward motif on the reverse complement DNA strand. As deamination occurs at only either C or A nucleotides, convention defines C and A variant motifs as forward motifs, and G and T variant motifs as reverse complement motifs.

Two naming schemes were utilized. Motifs which are associated with specific deaminases were labelled accordingly. The main deaminases that are known to be ubiquitous deaminases (i.e. found to be expressed in all or most tissue types) are AID, ADAR, APOBEC3G (abbreviated to A3G) and APOBEC3B (abbreviated to A3B).

The four primary deaminase motifs are as follows:
AID: WR-C-/-G-YW (written as AID_WR-C-);
ADAR: W-A-/-T-W (written as ADAR_W-A-);
APOBEC3G (A3G): C-C-/-G-G (written as A3G_C-C-); and
APOBEC3B (A3B): T-C-W/W-G-A (written as A3B_T-C-W).

SNVs at secondary deaminase motifs were also assessed. These secondary deaminase motifs included: AIDb: WR-C-G/C-G-YW; AIDc: WR-C-GS/SC-G-YW; AIDd: WR-C-Y/R-G-YW; AIDe: WR-C-GW/WC-G-YW; AIDh: WR-C-T/A-G-YW; ADARb: W-A-Y/R-T-W; ADAR: SW-A-Y/R-T-WS; ADARf: SW-A-/-T-WS; ADARh: W-A-S/S-T-W; ADARk: CW-A-/-T-WG; ADARn: -A-WA/TW-T-; ADARp: -A-WT/AW-T-; A3Gb: -C-G/C-G-; A3Gc: C-C-GW/WC-G-G; A3Ge:SC-C-GS/SC-G-GS; A3Gi: SG-C-G/C-G-CS; A3Gn: YYC-C-S/S-G-GRR; A3Go: TC-C-G/C-G-GA; A3Bf: ST-C-G/C-G-AS; A3BJ: RT-C-G/C-G-AY; A3F: T-C-/-G-A; and A1: -C-A/T-G-.

Motifs not known to be specifically associated with deaminase enzymes are labelled as “Gen” motifs; and ADAR_Gen is used to identify motifs where A or T is the targeted or mutated nucleotide (thus resulting in the variant or SNV):
2Gen1—two base motifs where the first position is the variant, e.g. 2Gen1_-C-T
3Gen1—three base motifs where the first position is the variant. e.g. 3Gen1_-C-TA
3Gen2—three base motifs where the second position is the variant, e.g. ADAR_3 Gen2_G-A-T
3Gen3—three base motifs where the third position is the variant, e.g. 3Gen3_GA-C-
4Gen3—four base motifs where the third position is the variant, e.g. ADAR_4 Gen3_AT-A-T

To determine the motif-associated metric, an assessment of the targeted nucleotide (i.e. whether the targeted nucleotide is an A, T, C or G), the type of SNV (e.g. whether the targeted nucleotide is now an A, T, G or C), whether the SNV is a transition or transversion SNV, whether the SNV is synonymous or non-synonymous, the motif in which the targeted nucleotide resides, the codon context of the SNV, and/or the strand on which the SNV occurs, was also performed.

3. Non-Motif Metrics

Metrics that are not associated with a motif were also assessed. These included metrics based on SNVs in the cds and metrics based on SNVs throughout the genome (i.e. cds and nc SNVs). Such metrics typically include “All” or “other” in the metric name.

### Example 2

### Cancer Progression Prediction Using Most Significant Metrics

Preliminary modelling was performed to identify 20 metrics for each cancer (ADCC, BLGG, LUSC, MESO, SARC and SKCM) that contributed most to various models that could distinguish patients with a relatively low progression free survival (PFS) time and patients with a relatively high PFS. These included:
(1) for MESO: cds:3Gen2_C-C-C MC3%, g:A3Bj_RT-C-G C>T+G>A g cds:3Gen3_GG-C-non-syn cds:A3Gb_-C-G G>A at MC2 motif %, cds:A3B_T-C-W MC1 cds:3Gen1_-C-GC MC2%, cds:A3Gb_-C-G G>A MC2 Hits, cds:A1_-C-A G>A at MC3 cds cds:3Gen3_AG-C-MC2 cds:ADAR_W-A-non-syn cds:A3Bj_RT-C-G Ti g:3Gen2_T-C-G C>T+G>A g cds:AIDe_WR-C-GW Hits, cds:3Gen2_A-C-G MC2 non-syn cds:A3Gi_SG-C-G non-syn g:ADAR_W-A-A>G+T>C cds:2Gen1_-C-C G>T at MC1 cds:A3Gi_SG-C-G MC2%, cds:A3Bf_ST-C-G Ti cds:ADAR_3 Gen2_G-A-C non-syn %;
(2) for ADCC: g:A3F_T-C-Hits, cds:3Gen1_-C-TG G non-syn cds:3Gen2_C-C-T MC3%, cds:All G total, g:3Gen1_-C-TC C>T+G>A g cds:3Gen3_CT-C-MC3%, cds:All G nc:A3G_C-C-C>T+G>A nc cds:A3B_T-C-W G>A motif %, cds:AIDc_WR-C-GS cds:3Gen1_-C-GT G>A motif %, cds:A3B_T-C-W MC3 non-syn cds:3Gen3_TG-C-G>A cds:ADAR_2 Gen2_G-T-MC2%, cds:3Gen3_TG-C-G Ti/Tv cds:4Gen3_TT-C-C cds:2Gen1_-C-C C>A cds:A3G_C-C-C>T at MC1 cds:AIDb_WR-C-G G non-syn cds:A3G_C-C-MC3%;
(3) for BLGG: g:CG total, cds:AIDc_WR-C-GS MC3%, cds:A3B_T-C-W G non-syn cds:AIDd_WR-C-Y g:AIDc_WR-C-GS Hits, cds:3Gen2_A-C-C non-syn g:3Gen3_GA-C-C>A+G>T g cds:2Gen2_G-C-Hits, cds:4Gen3_TA-C-C non-syn nc:2Gen2_A-C-C>T+G>A nc cds:Other MC3 C cds:3Gen2_T-C-G Ti/Tv g:3Gen2_A-C-C C>A+G>T g g:3Gen3_CA-C-C>T+G>A g cds:3Gen2_T-C-C MC1 g:ADAR_2 Gen1_-T-T A>T+T>A g:ADAR_2 Gen2_G-T-A>T+T>A g:2Gen1_-C-T cds:ADAR_3 Gen1_-A-CA cds:ADAR_2 Gen2_T-T-;
(4) for SARC: nc:ADARb_W-A-Y A>G+T>C nc g:ADARk_CW-A-A>G+T>C g %, cds:ADAR_3 Gen3_CA-A-Ti cds:A3G_C-C-G>T cds:4Gen3_TT-C-T cds:ADARc_SW-A-Y T>C cds nc:ADARc_SW-A-Y A>G+T>C nc cds:A3F_T-C-G>C g:C>A+G>T cds:2Gen1_-C-T MC3 non-syn %, nc:ADARb_W-A-Y %, cds:AIDd_WR-C-Y C>A cds cds:Primary Deaminase %, cds:4Gen3_CA-C-C MC1 g:C>G+G>C g:2Gen1_-C-T C>G+G>C g g:AIDh_WR-C-T C>A+G>T g cds:A3Ge_SC-C-GS cds:ADAR_3 Gen3_CT-A-A>G motif %, cds:ADARf_SW-A-MC2%;
(5) for LUSC: cds:ADARp_-A-WT A>G at MC2 cds cds:3Gen1_-C-TC C>T cds cds:AIDd_WR-C-Y G>C cds:ADAR_3 Gen3_AC-A-A>G cds cds:3Gen1_-C-CT C>T at MC2 cds cds:A3Go_TC-C-G MC1 non-syn cds:3Gen2_G-C-T C>A motif %, nc:2Gen1_-C-T C>A+G>T nc cds:ADAR_2 Gen2_A-T-A>C at MC1 motif %, cds:4Gen3_CA-C-C cds:A3Gn_YYC-C-S C>T at MC3 cds cds:3Gen1_-C-AG G Ti/Tv cds:ADARh_W-A-S T>C cds:3Gen1_-C-CC C>T at MC1 motif %, cds:2Gen1_-C-C C>T at MC1 g:ADAR_4 Gen3_AG-A-G A>C+T>G cds:4Gen3_CT-C-C C>T at MC1 cds:ADAR_3 Gen1_-A-CC A>G cds cds:A3Gn_YYC-C-S C>T cds:ADAR_W-A-T>C at MC2%; and
(6) for SKCM: cds:4Gen3_AG-C-T MC1 non-syn cds:All A non-syn cds:3Gen1_-C-CG G>A at MC3%, cds:3Gen3_TT-C-C>A at MC1 motif %, cds:A3Gc_C-C-GW C>T motif %, cds:ADAR_W-A-A>G at MC3%, cds:ADARp_-A-WT T>A motif %, cds:3Gen3_CT-C-G non-syn cds:3Gen2_T-C-T G>A at MC2%, cds:ADAR_3 Gen1_-A-AT Ti cds:All C Ti/Tv cds:3Gen1_-C-TC C>T at MC3%, cds:4Gen3_AG-C-T G>A at MC1 motif %, cds:3Gen1_-C-CA Ti C:G cds:3Gen3_AT-C-G>A at MC2%, cds:4Gen3_TG-C-T Ti C:G cds:ADAR_3 Gen2_C-A-C T>G at MC3 cds cds:4Gen3_GC-C-C C>T at MC2 cds:4Gen3_AC-C-T Ti/Tv cds:AIDh_WR-C-T G>A at MC2 cds %

The top metrics for each cancer were combined to form an exemplary panel of CPAS metrics. This panel of 142 metrics is set forth in Table D, above.

Patient cohorts obtained from the PanCancer Atlas for this analysis included Adrenocortical Carcinoma (ADCC), Brain Lower Grade Glioma (BLGG), Lung Squamous Cell Carcinoma (LUSC), Mesothelioma (MESO), Sarcoma (SARC), and Skin Cutaneous Melanoma (SKCM). Genomic data was obtained for those patients that had Progression Free Survival (PFS) recorded (n=1,295 in total; patients without PFS recorded were excluded). Genomic data was analysed as described above, generating output for the 142 metric panel set forth in Table D for each patient. For each cancer type (n=6), patients were categorized as “PFS_Low” or “PFS_High”. Patients in the “PFS_Low” category had recurrence or cancer progression before the predetermined time period (e.g. <24 months). Patients in the “PFS_High” category did not experience recurrence or progression before the time period (e.g. >24 months). For each patient cohort, computational models were trained to predict patient outcomes (“PFS_Low” or “PFS_High”) using the output from the panel of 142 metrics.

Computational models were trained on ˜75% of the patients for each cohort (“training data”), hyperparameters were tuned using ˜10% of the patients (“tuning data”), and predictions were made on the remaining ˜15% of patients, sequestered before analysis (“validation data”). While XGBoost modeling was used in the present study the nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like.

The overall accuracy, sensitivity and specificity for predictions made on the “validation” patients (those not used for training or tuning the model) are presented for each patient cohort (ADCC, BLGG, LUSC, MESO, SARC and SKCM). Genomic data was obtained for those patients that had Progression Free Survival (PFS). Kaplan-Meier curves are also presented for each cohort including log-rank statistical tests for comparison of PFS distributions (significance: p<0.05).

A. Modeling for MESO Patients

The TCGA PanCancer Atlas Mesothelioma cohort (MESO) contains 32 patients classified as “progressed” in less than 12 months (PFS<12 months and PFS status=“progressed”), and 38 patients that have a PFS of greater than or equal to 12 months (PFS>=12 months). A gradient boosting decision tree ensemble was generated and used to predict patient outcome in the ‘blind’ validation dataset. Table 1 sets forth the 21 metrics used in the model.

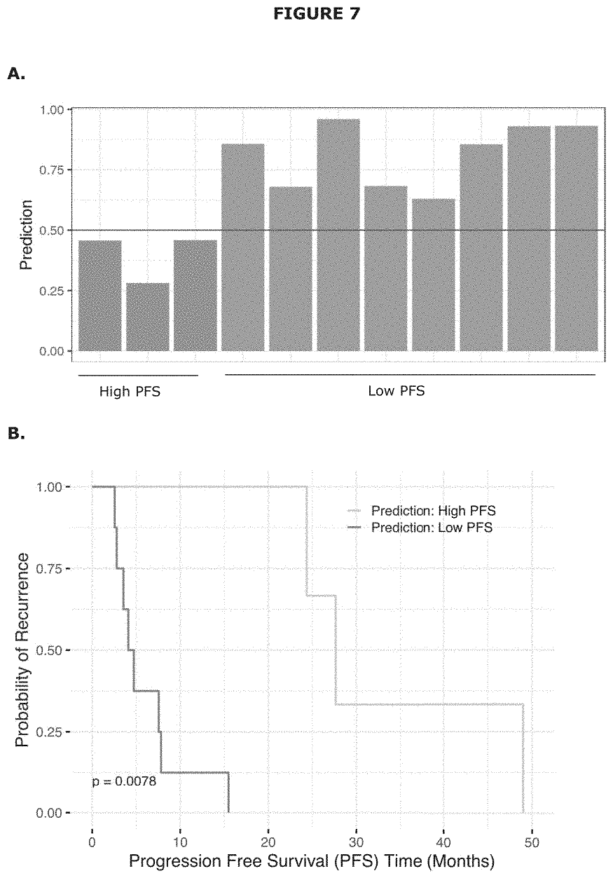

The overall accuracy of predictions was 100% (Accuracy: 100%, Sensitivity: 1.00, Specificity: 1.00): 100% of validation patients were correctly classified as “High_PFS” (3/3) and 100% were correctly classified as “Low_PFS” (8/8). The validation data was not used to train or tune the model. Kaplan-Meier curves, including log-rank statistical tests for comparison of PFS distributions (significance: p<0.05) are shown in FIG. 7.

B. Modeling for ADCC Patients

The TCGA PanCancer Atlas Adrenocortical Carcinoma cohort (ADCC) contains 39 patients classified as “progressed” in less than 24 months (PFS<24 months and PFS status=“progressed”), and 46 patients that have a PFS of greater than or equal to 24 months (PFS>=24 months). A gradient boosting decision tree ensemble was generated and was used to predict patient outcome in the ‘blind’ validation dataset. Table 1 sets forth the 38 metrics used in the model.

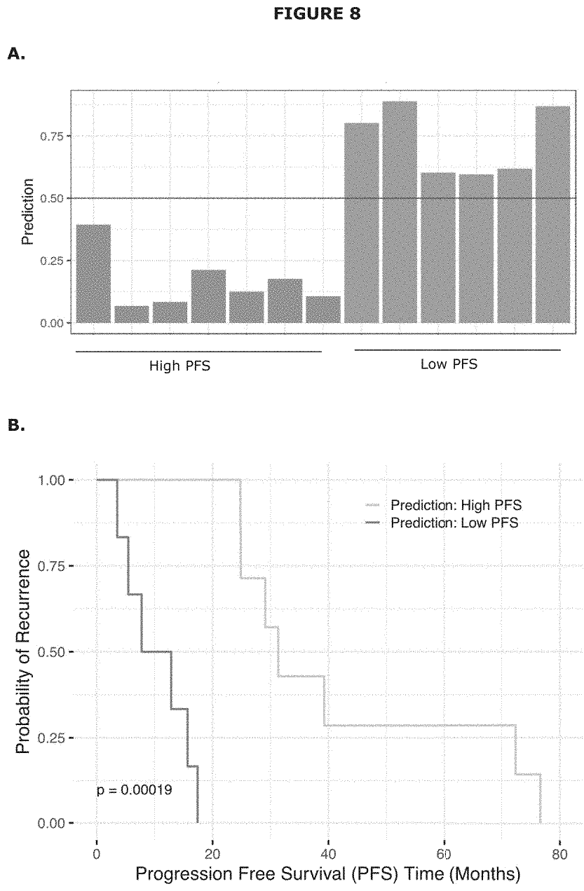

The overall accuracy of predictions was 100% (Accuracy: 100%, Sensitivity: 1.00, Specificity: 1.00): 100% of validation patients were correctly classified as “High_PFS” (7/7) and 100% were correctly classified as “Low_PFS” (6/6). The validation data was not used to train or tune the model. The validation data was not used to train or tune the model. Kaplan-Meier curves, including log-rank statistical tests for comparison of PFS distributions (significance: p<0.05) are shown in FIG. 8.

C. Modeling for BLGG Patients

The TCGA PanCancer Atlas Lower Grade Glioma cohort (BLGG) contains 122 patients classified as “progressed” in less than 24 months (PFS<24 months and PFS status=“progressed”), and 168 patients that have a PFS of greater than or equal to 24 months (PFS>=24 months). A gradient boosting decision tree ensemble was generated and was used to predict patient outcome in the ‘blind’ validation dataset. Table 1 sets forth the 88 metrics used in the model.

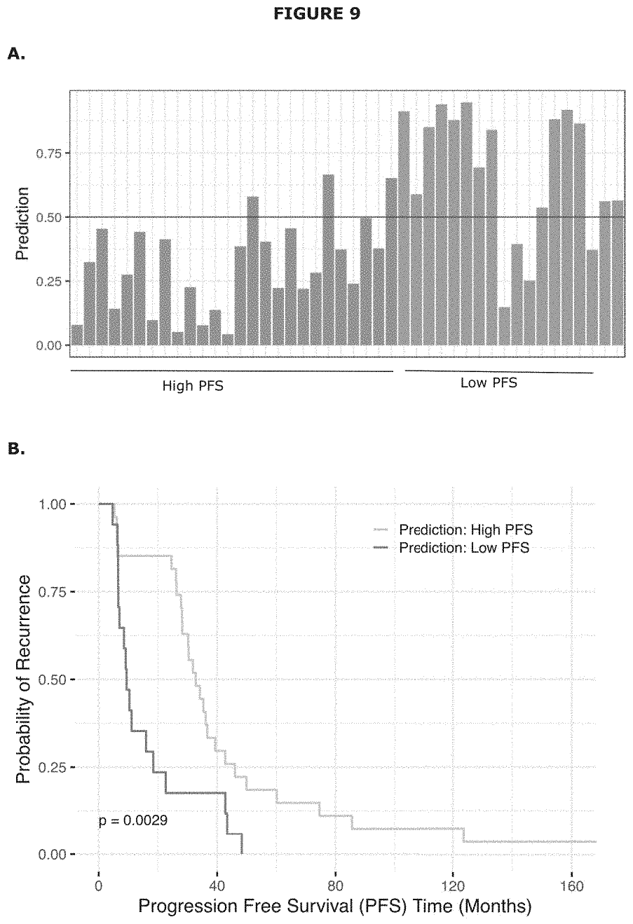

The overall accuracy of predictions was 84% (Accuracy: 84.09%, Sensitivity: 0.8846, Specificity: 0.7778): 88% of validation patients were correctly classified as “High_PFS” (23/26) and 77% were correctly classified as “Low_PFS” (14/18). The validation data was not used to train or tune the model. Kaplan-Meier curves, including log-rank statistical tests for comparison of PFS distributions (significance: p<0.05) are shown in FIG. 9.

D. Modeling for SARC Patients

The TCGA PanCancer Atlas Sarcoma cohort (SARC) contains 87 patients classified as “progressed” in less than 18 months (PFS<18 months and PFS status=“progressed”), and 98 patients that have a PFS of greater than or equal to 18 months (PFS>=18 months). A gradient boosting decision tree ensemble was generated and was used to predict patient outcome in the ‘blind’ validation dataset. Table 1 sets forth the 34 metrics used in the model.

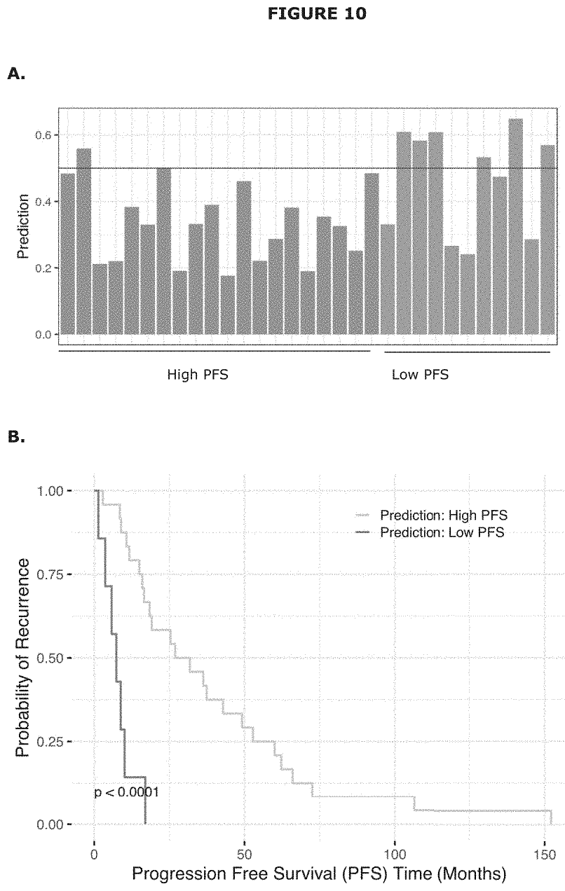

The overall accuracy of predictions was 81% (Accuracy: 80.65%, Sensitivity: 0.9500, Specificity: 0.5455): 95% of validation patients were correctly classified as “High_PFS” (19/20) and 54.55% were correctly classified as “Low_PFS” (6/11). The validation data was not used to train or tune the model. Kaplan-Meier curves, including log-rank statistical tests for comparison of PFS distributions (significance: p<0.05) are shown in FIG. 10.

E. Modeling for LUSC Patients

The TCGA PanCancer Atlas Lung Squamous Cell Carcinoma cohort (LUSC) contains 109 patients classified as “progressed” in less than 36 months (PFS<36 months and PFS status=“progressed”), and 125 patients that have a PFS of greater than or equal to 36 months (PFS>=36 months). A gradient boosting decision tree ensemble was generated and was used to predict patient outcome in the ‘blind’ validation dataset. Table 1 sets forth the 102 metrics used in the LUSC model.

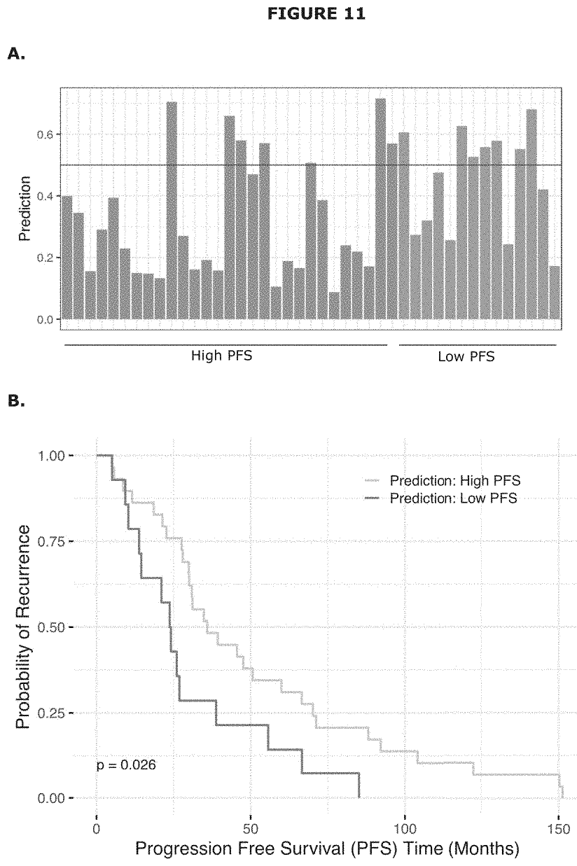

The overall accuracy of predictions was 67% (Accuracy: 67.44%, Sensitivity: 0.7586, Specificity: 0.500): 75.86% of validation patients were correctly classified as “High_PFS” (22/29) and 50% were correctly classified as “Low_PFS” (7/14). The validation data was not used to train or tune the model. Kaplan-Meier curves, including log-rank statistical tests for comparison of PFS distributions (significance: p<0.05) are shown in FIG. 11.

F. Modeling for SKCM Patients

The TCGA PanCancer Atlas Skin Cutaneous Melanoma (SKCM) contains 178 patients classified as “progressed” in less than 30 months (PFS<30 months and PFS status=“progressed”), and 180 patients that have a PFS of greater than or equal to 30 months (PFS>=30 months). A gradient boosting decision tree ensemble was generated and was used to predict patient outcome in the ‘blind’ validation dataset. Table 1 sets forth the 100 metrics used in the SKCM model.

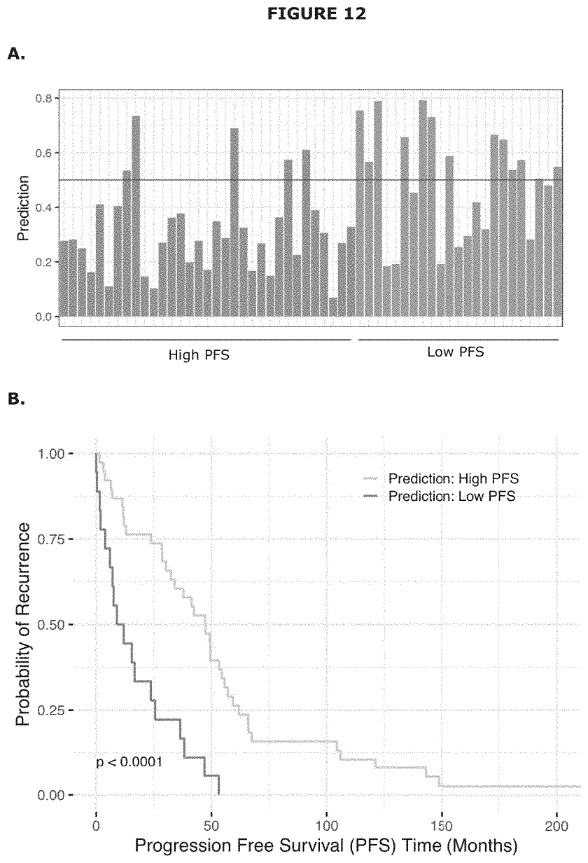

The overall accuracy of predictions was 73% (Accuracy: 73.21%, Sensitivity: 0.8485, Specificity: 0.5652): 84.85% of validation patients were correctly classified as “High_PFS” (28/33) and 56.52% were correctly classified as “Low_PFS” (13/23). The validation data was not used to train or tune the model. Kaplan-Meier curves, including log-rank statistical tests for comparison of PFS distributions (significance: p<0.05) are shown in FIG. 12.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

TABLE 1
Metrics used for each model

| MESO | ADCC | BLGG |
|---|---|---|
| cds: A3Bf_ST-C-G Ti % | cds: All G total | g: CG total |
| g: 3Gen2_T-C-G C > T + | cds: 3Gen1_-C-TG G non-syn % | cds: AIDd_WR-C-Y % |
| G > A g % | | |
| cds: 2Gen1_-C-C C > T | g: A3F_T-C- Hits | variants in VCF |
| at MC1% | | |
| cds: All C Ti/Tv % | cds: 3Gen3_GG-C- non-syn % | cds: 4Gen3_TA-C-C non- |
| | | syn % |
| g: 3Gen3_CA-C- C > | cds: 3Gen1_-C-GT G > A motif % | cds: 3Gen2_C-C-T MC3% |
| T + G > A g % | | |
| cds: 3Gen2_C-C-C MC3% | cds: A3Bj_RT-C-G Ti % | cds: AIDd_WR-C-Y G > C % |
| cds: A3Gn_YYC-C-S C > T % | cds: 3Gen2_C-C-T MC3% | cds: A3Gb_-C-G MC1% |
| cds: A3G_C-C- MC3% | nc: A3G_C-C- C > T + | g: 3Gen2_T-C-G C > T + |
| | G > A nc % | G > A g % |
| cds: 3Gen3_GG-C- | cds: AIDd_WR-C-Y % | cds: A3B_T-C-W G non-syn % |
| non-syn % | | |
| g: 3Gen2_A-C-C C > | cds: 3Gen1_-C-TC C > T cds % | g: 3Gen3_GA-C- C > A + |
| A + G > T g % | | G > T g % |
| cds: 4Gen3_TT-C-C % | cds: A3B_T-C-W G > A motif % | cds: 2Gen2_G-C- Hits |
| cds: 3Gen2_C-C-T MC3% | g: CG total | cds: AIDc_WR-C-GS MC3% |
| g: 2Gen1_-C-T C > | cds: A3G_C-C- MC3% | cds: All G total |
| G + G > Cg % | | |
| cds: Primary Deaminase % | cds: AIDb_WR-C-G G non-syn % | cds: All A non-syn % |
| cds: A3Gb_-C-G G > A | cds: A3G_C-C- C > T at MC1% | cds: ADAR_2Gen2_T-T- % |
| at MC2 motif % | | |
| cds: 4Gen3_CA-C-C % | cds: 3Gen3_TG-C- G > A % | cds: 3Gen2_A-C-C non-syn % |
| cds: A3G_C-C- G > T % | g: 3Gen3_GA-C- C > A + | g: 3Gen3_CA-C- C > T + |
| | G > T g % | G > A g % |
| cds: A3Gi_SG-C-G | cds: 3Gen2_A-C-G MC2 | g: ADARk_CW-A- A > G + |
| non-syn % | non-syn % | T > C g % |
| g: C > G + G > C % | cds: 3Gen3_CT-C- MC3% | nc: ADARb_W-A-Y A > G + |
| | | T > C nc % |
| cds: Other MC3% | cds: ADAR_2Gen 2_G-T- MC2% | g: 2Gen1_-C-T % |
| cds: A3B_T-C-W G > A | cds: ADAR_3Gen 3_CA- | cds: Other MC3 C % |
| motif % | A- Ti % | |
| | g: AIDh_WR-C-T C > A + | g: 2Gen1_-C-T C > G + |
| | G > T g % | G > C g % |
| | cds: A3B_T-C-W MC3 non-syn % | cds: ADAR_W-A- non-syn % |
| | cds: 2Gen1_-C-C C > A % | g: 3Gen2_A-C-C C > A + |
| | | G > T g % |
| | cds: A1_-C-A G > A at | g: ADAR_2Gen2_G-T- |
| | MC3 cds % | A > T + T > A % |
| | cds: 3Gen1_-C-CA Ti C:G % | cds: A3G_C-C- C > T at |
| | | MC1% |
| | cds: ADAR_W-A- non-syn % | cds: 3Gen1_-C-GC MC2% |
| | cds: 3Gen1_-C-CA Ti % | cds: 3Gen2_G-C-T % |
| | cds: All G % | cds: A3F_T-C- G > C % |
| | g: 3Gen2_T-C-G C > T + | g: 4Gen3_GG-C-G C > T + |
| | G > A g % | G > A g % |
| | cds: A3Gb_-C-G MC1% | cds: A3Gb_-C-G G > A at |
| | | MC2 motif % |
| | cds: A3B_T-C-W G non-syn % | cds: ADARb_W-A-Y MC2% |
| | nc: 2Gen2_A-C- C > T + | cds: All G % |
| | G > A nc % | |
| | cds: A3Gi_SG-C-G non-syn % | g: A3F_T-C- Hits |
| | cds: Other G MC3 Ti/Tv % | cds: 3Gen2_T-C-C MC1% |
| | cds: A3Gb_-C-G G > A at | cds: A3B_T-C-W Ti % |
| | MC2 motif % | |
| | cds: A3B_T-C-W Ti % | cds: ADAR_3Gen1_-A-AT |
| | | Ti % |
| | g: 2Gen1_-C-T % | cds: ADARh_W-A-S T > C % |
| | | cds: A3Gn_YYC-C-S C > T % |
| | | cds: A3Ge_SC-C-GS % |
| | | cds: 2Gen2_A-C- MC3% |
| | | cds: ADAR_2Gen2_G-T- |
| | | MC2% |
| | | cds: ADAR_3Gen3_CA-A- |
| | | Ti % |
| | | cds: Primary Deaminase % |
| | | g: C > G + G > C % |
| | | cds: A3Bf_ST-C-G Ti % |
| | | cds: 3Gen3_CT-C- MC3% |
| | | cds: A3Gi_SG-C-G non- syn % |
| | | cds: Other MC3% |
| | | cds: ADAR_3Gen1_-A-CA % |
| | | cds: A3F_T-C- C > A % |
| | | cds: 2Gen1_-C-C C > T at MC1% |
| | | cds: A3Gc_C-C-GW C > T motif % |
| | | cds: AIDc_WR-C-GS % |
| | | g: ADAR_2Gen1_-T-T |
| | | A > T + T > A % |
| | | cds: A3B_T-C-W MC1% |
| | | cds: ADAR_3Gen2_G-A-C non-syn % |
| | | cds: 2Gen1_-C-C C > A % |
| | | cds: 3Gen1_-C-GT G > A motif % |
| | | cds: A3Bj_RT-C-G Ti % |
| | | g: 3Gen1_-C-TC C > T + |
| | | G > A g % |
| | | g: C > A + G > T % |
| | | cds: 3Gen2_A-C-C MC2% |
| | | cds: 2Gen1_-C-C MC2% |
| | | g: 3Gen2_G-C-T % |
| | | g: A3Bj_RT-C-G C > T + |
| | | G > A g % |
| | | g: ADAR_W-A- A > G + |
| | | T > C % |
| | | cds: 3Gen3_AT-C- C:G % |
| | | cds: 3Gen1_-C-TG G non-syn % |
| | | cds: Other G MC3 Ti/Tv % |
| | | cds: A3Gb_-C-G G > A |
| | | MC2 Hits |
| | | cds: 3Gen1_-C-TC C > T cds % |
| | | cds: 2Gen1_-C-T MC3 non-syn % |
| | | cds: AIDb_WR-C-G G non-syn % |
| | | g: AIDc_WR-C-GS Hits |
| | | cds: 3Gen2_T-C-C MC3% |
| | | cds: 3Gen2_T-C-G Ti/Tv % |
| | | cds: A1_-C-A G > A at |
| | | MC3 cds % |
| | | nc: A3G_C-C- C > T + |
| | | G > A nc % |
| | | nc: 2Gen2_A-C- C > T + |
| | | G > A nc % |
| | | cds: 3Gen3_TG-C- G |
| | | Ti/Tv % |
| | | cds: 3Gen1_-C-CA Ti % |
| | | cds: 3Gen3_TG-C- G > A % |
| | | cds: 3Gen3_CT-C- G non-syn % |
| | | cds: All C Ti/Tv % |
| | | cds: A3G_C-C- MC3% |
| | | cds: ADARc_SW-A-Y MC2% |
| | | cds: 3Gen3_GG-C- non-syn % |
| SARC | LUSC | SKCM |
| cds: Other MC3 C % | cds: 3Gen1_-C-CC | cds: 4Gen3_AG-C-T |
| | C > T at MC1 motif % | MC1 non-syn % |
| nc: ADARb_W-A-Y A > G + | cds: 3Gen1_-C-CT | cds: 3Gen1_-C-CG G > A |
| T > C nc % | C > T at MC2 cds % | at MC3% |
| cds: 4Gen3_TT-C-T % | cds: ADARp_-A-WT | cds: 4Gen3_AC-C-T |
| | A > G at MC2 cds % | Ti/Tv % |
| g: ADARk_CW-A- A > G + | cds: Other MC3 C % | g: C > G + G > C % |
| T > C g % | | |
| g: ADARn_-A-WA A > G + | cds: Other MC3% | cds: A3B_T-C-W MC3 |
| T > C % | | non-syn % |
| cds: A3G_C-C- G > T % | cds: A3Gb_-C-G MC1% | cds: All A non-syn % |
| cds: A3Gb_-C-G MC1% | g: 3Gen1_-C-TC C > T + | cds: 3Gen3_AG-C- MC2% |
| | G > A g % | |
| nc: ADARb_W-A-Y % | cds: ADAR_W-A- A > G | cds: A3B_T-C-W MC1% |
| | at MC3% | |
| cds: A3Ge_SC-C-GS % | cds: ADAR_W-A- non- | cds: ADAR_3Gen2_C-A- |
| | syn % | C T > G at MC3 cds % |
| cds: Primary Deaminase % | cds: ADAR_3Gen3_AC- | cds: 3Gen1_-C-TC C > T |
| | A- A > G cds % | at MC3% |
| cds: ADAR_2Gen2_G-T- | cds: 2Gen1_-C-C C > A | cds: 4Gen3_GC-C-C |
| MC2% | % | C > T at MC2% |
| g: 4Gen3_GG-C-G C > T + | cds: ADARf_SW-A- | cds: All C Ti/Tv % |
| G > A g % | MC2% | |
| cds: 2Gen1_-C-C MC2% | g: ADAR_2Gen2_G-T- | cds: A3Bj_RT-C-G Ti % |
| | A > T + T > A % | |
| cds: 3Gen1_-C-GT G > A | cds: 4Gen3_GC-C-A % | cds: AIDh_WR-C-T G > A |
| motif % | | at MC2 cds % |
| cds: A3Gn_YYC-C-S | cds: A3Go_TC-C-G | cds: 4Gen3_TT-C-C % |
| C > T % | MC1 non-syn % | |
| cds: 2Gen1_-C-C C > T at | g: 3Gen2_G-C-T % | cds: 3Gen1_-C-CC C > T |
| MC1% | | at MC1 motif % |
| cds: A3B_T-C-W MC3 | cds: A3G_C-C- C > T at | cds: ADAR_2Gen2_T-T- |
| non-syn % | MC1% | % |
| cds: AIDd_WR-C-Y % | cds: AIDc_WR-C-GS | cds: 3Gen2_T-C-C MC1 |
| | MC3% | % |
| g: 3Gen3_CA-C- C > T + | cds: 3Gen1_-C-GT | cds: All G % |
| G > A g % | G > A motif % | |
| cds: All A non-syn % | nc: 2Gen1_-C-T C > A + | cds: ADAR_W-A- A > G |
| | G > T nc % | at MC3% |
| g: 2Gen1_-C-T C > G + | cds: ADARc_SW-A-Y | cds: A3G_C-C- MC3% |
| G > C g % | MC2% | |
| cds: ADARb_W-A-Y MC2% | cds: ADARh_W-A-S | cds: Other MC3 C % |
| | T > C % | |
| cds: All G % | cds: 2Gen1_-C-C C > T | g: 3Gen2_A-C-C C > A + |
| | at MC1% | G > T g % |
| g: A3Bj_RT-C-G C > T + | g: ADAR_2Gen1_-T-T | cds: ADARc_SW-A-Y |
| G > A g % | A > T + T > A % | MC2% |
| cds: A3Gn_YYC-C-S | cds: AIDd_WR-C-Y | cds: 3Gen1_-C-CA Ti |
| C > T at MC3 cds % | C > A cds % | C:G % |
| cds: A3B_T-C-W G non- | nc: A3G_C-C- C > T + | cds: 3Gen1_-C-TC C > T |
| syn % | G > A nc % | cds % |
| cds: A3G_C-C- MC3% | cds: A3Gc_C-C-GW | cds: 3Gen2_C-C-C MC3% |
| | C > T motif % | |
| cds: All G total | cds: ADAR_3Gen1_-A- | cds: 3Gen3_CT-C- C > T |
| | AT Ti % | at MC1 motif % |
| cds: CDS Variants | cds: 3Gen3_CT-C- | g: ADAR_4Gen3_AG-A- |
| | MC3% | G A > C + T > G % |
| g: CG total | cds: 4Gen3_CT-C-C | cds: 3Gen3_CT-C- G |
| | C > T at MC1% | non-syn % |
| g: 3Gen2_T-C-G C > T + | cds: 3Gen2_T-C-C | cds: 3Gen2_A-C-C non- |
| G > A g % | MC1% | syn % |
| cds: A3B_T-C-W MC1% | cds: A3G_C-C- G > T % | cds: 2Gen2_A-C- MC3% |
| cds: ADAR_3Gen3_CA- | cds: 3Gen1_-C-CA Ti | cds: 3Gen2_A-C-C MC2% |
| A- Ti % | % | |
| cds: AIDc_WR-C-GS % | cds: 3Gen1_-C-TG G | g: 3Gen1_-C-TC C > T + |
| | non-syn % | G > A g % |
| | cds: 3Gen2_A-C-C | cds: 3Gen2_T-C-T G > A |
| | non-syn % | at MC2% |
| | g: 2Gen1_-C-T C > G + | cds: 2Gen1_-C-C C > T |
| | G > C g % | at MC1% |
| | cds: All A non-syn % | cds: AIDb_WR-C-G G |
| | | non-syn % |
| | cds: A3Gi_SG-C-G | cds: A3Gb_-C-G MC1% |
| | MC2% | |
| | cds: Primary | cds: 2Gen1_-C-C C > A |
| | Deaminase % | % |
| | cds: 4Gen3_TT-C-T % | cds: A3Ge_SC-C-GS % |
| | g: A3Bj_RT-C-G C > T + | g: ADARn_-A-WA A > G + |
| | G > A g % | T > C % |
| | cds: 3Gen2_T-C-C | g: ADAR_W-A- A > G + |
| | MC3% | T > C % |
| | cds: 4Gen3_TT-C-C % | g : ADAR_2Gen2_G-T- |
| | | A > T + T > A % |
| | cds: 3Gen1_-C-CA Ti | g: AIDh_WR-C-T C > A + |
| | C:G % | G > T g % |
| | cds: A1_-C-A G > A at | cds: 4Gen3_TG-C-T Ti |
| | MC3 cds % | C:G % |
| | cds: A3Gb_-C-G G > A | cds: 3Gen2_G-C-T C:G |
| | at MC2 motif % | % |
| | cds: 3Gen3_CT-C- G | cds: 3Gen2_T-C-C MC3% |
| | non-syn % | |
| | cds: 3Gen2_G-C-T C:G | nc: ADARb_W-A-Y % |
| | % | |
| | cds: A3Ge_SC-C-GS % | cds: ADAR_3Gen2_G-A- |
| | | C non-syn % |
| | cds: 3Gen3_TG-C- | cds: ADAR_3Gen1_-A- |
| | G > A % | AT Ti % |
| | g: C > A + G > T % | g: ADARk_CW-A- A > G |
| | | + T > C g % |
| | cds: 4Gen3_CA-C-C % | cds: 3Gen1_-C-GC MC2% |
| | cds: AIDd_WR-C-Y | cds: 4Gen3_TA-C-C |
| | G > C % | non-syn % |
| | cds: All G % | g: 3Gen3_CA-C- C > T + |
| | | G > A g % |
| | cds: 3Gen3_TT-C- C > A | cds: 3Gen1_-C-AG G |
| | at MC1 motif % | Ti/Tv % |
| | g: AIDh_WR-C-T C > A + | cds: AIDc_WR-C-GS % |
| | G > T g % | |
| | g: 4Gen3_GG-C-G C > T + | cds: A3Gn_YYC-C-S |
| | G > A g % | C > T at MC3 cds % |
| | cds: 3Gen2_G-C-T | cds: 2Gen1_-C-C MC2 |
| | C > A motif % | % |
| | nc: ADARc_SW-A-Y | cds: 3Gen3_GG-C- non- |
| | A > G + T > C nc % | syn % |
| | g: 3Gen2_A-C-C C > A + | g: 2Gen1_-C-T C > G + |
| | G > T g % | G > C g % |
| | cds: A3B_T-C-W Ti % | cds: A1_-C-A G > A at |
| | | MC3 cds % |
| | g: 3Gen3_GA-C- C > A + | cds: A3G_C-C- C > T at |
| | G > T g % | MC1% |
| | cds: 3Gen3_CT-C- | nc: ADARc_SW-A-Y |
| | C > T at MC1 motif % | A > G + T > C nc % |
| | cds: ADAR_3Gen1_-A- | cds: ADAR_W-A-T > C at |
| | CC A > G cds % | MC2% |
| | cds: 3Gen1_-C-TC | cds: A3Go_TC-C-G MC1 |
| | C > T cds % | non-syn % |
| | cds: 4Gen3_CA-C-C | cds: 3Gen3_AT-C- C:G |
| | MC1% | % |
| | cds: 3Gen2_G-C-T % | cds: ADARh_W-A-S T > C |
| | | % |
| | nc: 2Gen2_A-C- C > T + | cds: A3G_C-C- G > T % |
| | G > A nc % | |
| | cds: 3Gen2_A-C-C | cds: ADARf_SW-A- MC2% |
| | MC2% | |
| | cds: A3F_T-C- C > A % | cds: ADAR_W-A- non- |
| | | syn % |
| | cds: CDS Variants | cds: ADARp_-A-WT T > A |
| | | motif % |
| | cds: ADAR_3Gen3_CA- | cds: 4Gen3_AG-C-T |
| | A- Ti % | G > A at MC1 motif % |
| | cds: 3Gen3_GG-C- | cds: ADAR_3Gen1_-A- |
| | non-syn % | CA % |
| | cds: ADARb_W-A-Y | cds: 3Gen2_C-C-T MC3% |
| | MC2% | |
| | g: ADAR_W-A- A > G + | cds: 3Gen1_-C-CT C > T |
| | T > C % | at MC2 cds % |
| | cds: 3Gen3_AT-C- C:G | cds: A3B_T-C-W Ti % |
| | % | |
| | cds: 2Gen1_-C-C G > T | g: 2Gen1_-C-T % |
| | at MC1% | |
| | cds: A3G_C-C- MC3% | cds: AIDc_WR-C-GS |
| | | MC3% |
| | cds: 3Gen2_C-C-C | cds: AIDe_WR-C-GW |
| | MC3% | Hits |
| | cds: A3B_T-C-W G > A | cds: AIDd_WR-C-Y C > A |
| | motif % | cds % |
| | cds: A3F_T-C- G > C % | cds: ADARb_W-A-Y MC2 |
| | | % |
| | cds: ADAR_2Gen2_G- | cds: A3Gc_C-C-GW C > T |
| | T- MC2% | motif % |
| | cds: 3Gen1_-C-AG G | cds: 2Gen1_-C-C G > T |
| | Ti/Tv % | at MC1% |
| | cds: A3Bj_RT-C-G Ti % | cds: 3Gen1_-C-CA Ti % |
| | nc: ADARb_W-A-Y | cds: Other G MC3 Ti/Tv |
| | A > G + T > C nc % | % |
| | cds: ADAR_2Gen2_T- | cds: CDS Variants |
| | T- % | |
| | g: 2Gen1_-C-T % | cds: ADAR_3Gen1_-A- |
| | | CC A > G cds % |
| | cds: 4Gen3_AC-C-T | cds: A3Gn_YYC-C-S |
| | Ti/Tv % | C > T % |
| | cds: A3Gi_SG-C-G | cds: A3Bf_ST-C-G Ti % |
| | non-syn % | |
| | cds: A3Bf_ST-C-G Ti % | cds: 2Gen2_G-C- Hits |
| | g: ADARk_CW-A- A > G + | cds: AIDd_WR-C-Y % |
| | T > C g % | |
| | cds: 3Gen1_-C-GC | cds: A3F_T-C- G > C % |
| | MC2% | |
| | g: 3Gen3_CA-C- C > T + | cds: 4Gen3_CT-C-C |
| | G > A g % | C > T at MC1% |
| | cds: 2Gen2_A-C- MC3% | cds: AIDd_WR-C-Y G > C |
| | | % |
| | variants in VCF | cds: A3Gi_SG-C-G MC2% |
| | cds: 4Gen3_AG-C-T | cds: Other MC3% |
| | MC1 non-syn % | |
| | g: 3Gen2_T-C-G C > T + | nc: 2Gen1_-C-T C > A + |
| | G > A g % | G > T nc % |
| | cds: A3Gn_YYC-C-S | cds: 3Gen2_G-C-T % |
| | C > T at MC3 cds % | |
| | cds: ADAR_3Gen1_-A- | g: 3Gen2_T-C-G C > T + |
| | CA % | G > A g % |
| | cds: 4Gen3_TA-C-C | cds: ADARc_SW-A-Y |
| | non-syn % | T > C cds % |
| | cds: All C Ti/Tv % | |
| | cds: ADARc_SW-A-Y | |
| | T > C cds % | |

1. A method for determining the likelihood that a cancer in a subject will progress or recur, the method comprising:
analyzing the sequence of a nucleic acid molecule from a subject with cancer to detect single nucleotide variations (SNVs) within the nucleic acid molecule;
determining a plurality of metrics based on the number and/or type of SNVs detected so as to obtain a subject profile of metrics; and,
determining the likelihood that the cancer will progress or recur based on a comparison between the subject profile and a reference profile of metrics;
wherein:
the plurality of metrics comprises 5 or more metrics selected from the metrics set forth in Table D and metrics related to the metrics set forth in Table D.

2. The method of claim 1, wherein the reference profile is representative of a cancer that is likely to progress or recur.

3. The method of claim 1, wherein the reference profile is representative of a cancer that is unlikely to progress or recur.

4. The method of claim 1, wherein the plurality of metrics comprises at least 10, 15, 20, 35, 30, 40, 45 or 50 metrics selected from selected from the metrics set forth in Table D and metrics related to the metrics set forth in Table D.

5. The method of claim 1, wherein the cancer is selected from among adrenal cancer, breast cancer, brain cancer, prostate cancer, liver cancer, colon cancer, stomach cancer, pancreatic cancer, skin cancer, thyroid, cervical cancer, lymphoid cancer, hematopoietic cancer, bladder cancer, lung cancer, renal cancer, rectal cancer, ovarian cancer, uterine cancer, head and neck cancer, mesothelioma and sarcoma.

6. The method of claim 1, wherein the cancer is:
(a) the cancer is mesothelioma and the plurality of metrics comprises least or about 5 metrics selected from cds:A3Bf_ST-C-G Ti %; g:3Gen2_T-C-G C>T+G>A g %; cds:2Gen1-C-C C>T at MC1%; cds:All C Ti/Tv %; g:3Gen3_CA-C-C>T+G>A g %; cds:3Gen2_C-C-C MC3%; cds:A3Gn_YYC-C-S C>T %; cds:A3G_C-C-MC3%; cds:3Gen3_GG-C-non-syn %; g:3Gen2_A-C-C C>A+G>T g %; cds:4Gen3_TT-C-C %; cds:3Gen2_C-C-T MC3%; g:2Gen1_-C-T C>G+G>C g %; cds:Primary Deaminase %; cds:A3Gb_-C-G G>A at MC2 motif %; cds:4Gen3_CA-C-C %; cds:A3G_C-C-G>T %; cds:A3Gi_SG-C-G non-syn %; g:C>G+G>C %; cds:Other MC3%; cds:A3B_T-C-W G>A motif %, and related metrics thereto;
(b) the cancer is adrenocortical carcinoma and the plurality of metrics comprises least or about 5 metrics selected from cds:All G total; cds:3Gen1_-C-TG G non-syn %; g:A3F_T-C-Hits; cds:3Gen3_GG-C-non-syn %; cds:3Gen1_-C-GT G>A motif %; cds:A3Bj_RT-C-G Ti %; cds:3Gen2_C-C-T MC3%; nc:A3G_C-C-C>T+G>A nc %; cds:AIDd_WR-C-Y %; cds:3Gen1_-C-TC C>T cds %; cds:A3B_T-C-W G>A motif %; g:CG total; cds:A3G_C-C-MC3%; cds:AIDb_WR-C-G G non-syn %; cds:A3G_C-C-C>T at MC1%; cds:3Gen3_TG-C-G>A %; g:3Gen3_GA-C-C>A+G>T g %; cds:3Gen2_A-C-G MC2 non-syn %; cds:3Gen3_CT-C-MC3%; cds:ADAR 2Gen2_G-T-MC2%; cds:ADAR_3Gen3_CA-A-Ti %; g:AIDh_WR-C-T C>A+G>T g %; cds:A3B_T-C-W MC3 non-syn %; cds:2Gen1_-C-C C>A %; cds:A1_-C-A G>A at MC3 cds %; cds:3Gen1_-C-CA Ti C:G %; cds:ADAR_W-A-non-syn %; cds:3Gen1_-C-CA Ti %; cds:All G %; g:3Gen2_T-C-G C>T+G>A g %; cds:A3Gb_-C-G MC1%; cds:A3B_T-C-W G non-syn %; nc:2Gen2_A-C-C>T+G>A nc %; cds:A3Gi_SG-C-G non-syn %; cds:Other G MC3 Ti/Tv %; cds:A3Gb_-C-G G>A at MC2 motif %; cds:A3B_T-C-W Ti %; and g:2Gen1_-C-T %, and related metrics thereto;
(c) the cancer is brain cancer and the plurality of metrics comprises least or about 5 metrics selected from g:CG total; cds:AIDd_WR-C-Y %; variants in VCF; cds:4Gen3_TA-C-C non-syn %; cds:3Gen2_C-C-T MC3%; cds:AIDd_WR-C-Y G>C %; cds:A3Gb_-C-G MC1%; g:3Gen2_T-C-G C>T+G>A g %; cds:A3B_T-C-W G non-syn %; g:3Gen3_GA-C-C>A+G>T g %; cds:2Gen2_G-C-Hits; cds:AIDc_WR-C-GS MC3%; cds:All G total; cds:All A non-syn %; cds:ADAR_2Gen2_T-T-%; cds:3Gen2_A-C-C non-syn %; g:3Gen3_CA-C-C>T+G>A g %; g:ADARk_CW-A-A>G+T>C g %; nc:ADARb_W-A-Y A>G+T>C nc %; g:2Gen1_-C-T %; cds:Other MC3 C %; g:2Gen1_-C-T C>G+G>C g %; cds:ADAR_W-A-non-syn %; g:3Gen2_A-C-C C>A+G>T g %; g:ADAR_2Gen2_G-T-A>T+T>A %; cds:A3G_C-C-C>T at MC1%; cds:3Gen1_-C-GC MC2%; cds:3Gen2_G-C-T %; cds:A3F_T-C-G>C %; g:4Gen3_GG-C-G C>T+G>A g %; cds:A3Gb_-C-G G>A at MC2 motif %; cds:ADARb_W-A-Y MC2%; cds:All G %; g:A3F_T-C-Hits; cds:3Gen2_T-C-C MC1%; cds:A3B_T-C-W Ti %; cds:ADAR 3_Gen1_-A-AT Ti %; cds:ADARh_W-A-S T>C %; cds:A3Gn_YYC-C-S C>T %; cds:A3Ge_SC-C-GS %; cds:2Gen2_A-C-MC3%; cds:ADAR_2Gen2_G-T-MC2%; cds:ADAR_3Gen3_CA-A-Ti %; cds:Primary Deaminase %; g:C>G+G>C %; cds:A3Bf_ST-C-G Ti %; cds:3Gen3_CT-C-MC3%; cds:A3Gi_SG-C-G non-syn %; cds:Other MC3%; cds:ADAR_3Gen1_-A-CA %; cds:A3F_T-C-C>A %; cds:2Gen1_-C-C C>T at MC1%; cds:A3Gc_C-C-GW C>T motif %; cds:AIDc_WR-C-GS %; g:ADAR_2Gen1_-T-T A>T+T>A %; cds:A3B_T-C-W MC1%; cds:ADAR_3Gen2_G-A-C non-syn %; cds:2Gen1_-C-C C>A %; cds:3Gen1_-C-GT G>A motif %; cds:A3Bj_RT-C-G Ti %; g:3Gen1_-C-TC C>T+G>A g %; g:C>A+G>T %; cds:3Gen2_A-C-C MC2%; cds:2Gen1_-C-C MC2%; g:3Gen2_G-C-T %; g:A3Bj_RT-C-G C>T+G>A g %; g:ADAR_W-A-A>G+T>C %; cds:3Gen3_AT-C-C:G %; cds:3Gen1_-C-TG G non-syn %; cds:Other G MC3 Ti/Tv %; cds:A3Gb_-C-G G>A MC2 Hits; cds:3Gen1_-C-TC C>T cds %; cds:2Gen1_-C-T MC3 non-syn %; cds:AIDb_WR-C-G G non-syn %; g:AIDc_WR-C-GS Hits; cds:3Gen2_T-C-C MC3%; cds:3Gen2_T-C-G Ti/Tv %; cds:A1_-C-A G>A at MC3 cds %; nc:A3G_C-C-C>T+G>A nc %; nc:2Gen2_A-C-C>T+G>A nc %; cds:3Gen3_TG-C-G Ti/Tv %; cds:3Gen1_-C-CA Ti %; cds:3Gen3_TG-C-G>A %; cds:3Gen3_CT-C-G non-syn %; cds:All C Ti/Tv %; cds:A3G_C-C-MC3%; cds:ADARc_SW-A-Y MC2%; and cds:3Gen3_GG-C-non-syn %, and related metrics thereto;
(d) the cancer is sarcoma and the plurality of metrics comprises least or about 5 metrics selected from cds:Other MC3 C %; nc:ADARb_W-A-Y A>G+T>C nc %; cds:4Gen3_TT-C-T %; g:ADARk_CW-A-A>G+T>C g %; g:ADARn_-A-WA A>G+T>C %; cds:A3G_C-C-G>T %; cds:A3Gb_-C-G MC1%; nc:ADARb_W-A-Y %; cds:A3Ge_SC-C-GS %; cds:Primary Deaminase %; cds:ADAR_2Gen2_G-T-MC2%; g:4Gen3_GG-C-G C>T+G>A g %; cds:2Gen1_-C-C MC2%; cds:3Gen1_-C-GT G>A motif %; cds:A3Gn_YYC-C-S C>T %; cds:2Gen1_-C-C C>T at MC1%; cds:A3B_T-C-W MC3 non-syn %; cds:AIDd_WR-C-Y %; g:3Gen3_CA-C-C>T+G>A g %; cds:All A non-syn %; g:2Gen1_-C-T C>G+G>C g %; cds:ADARb_W-A-Y MC2%; cds:All G %; g:A3Bj_RT-C-G C>T+G>A g %; cds:A3Gn_YYC-C-S C>T at MC3 cds %; cds:A3B_T-C-W G non-syn %; cds:A3G_C-C-MC3%; cds:All G total; cds:CDS Variants; g:CG total; g:3Gen2_T-C-G C>T+G>A g %; cds:A3B_T-C-W MC1%; cds:ADAR_3Gen3_CA-A-Ti %; cds:AIDc_WR-C-GS %, and related metrics thereto;
(e) the cancer is lung cancer and the plurality of metrics comprises least or about 5 metrics selected from cds:3Gen1_-C-CC C>T at MC1 motif %; cds:3Gen1_-C-CT C>T at MC2 cds %; cds:ADARp-A-WT A>G at MC2 cds %; cds:Other MC3 C %; cds:Other MC3%; cds:A3Gb_-C-G MC1%; g:3Gen1_-C-TC C>T+G>A g %; cds:ADAR_W-A-A>G at MC3%; cds:ADAR_W-A-non-syn %; cds:ADAR 3Gen3_AC-A-A>G cds %; cds:2Gen1_-C-C C>A %; cds:ADARf_SW-A-MC2%; g:ADAR_2Gen2_G-T-A>T+T>A %; cds:4Gen3_GC-C-A %; cds:A3Go_TC-C-G MC1 non-syn %; g:3Gen2_G-C-T %; cds:A3G_C-C-C>T at MC1%; cds:AIDc_WR-C-GS MC3%; cds:3Gen1_-C-GT G>A motif %; nc:2Gen1_-C-T C>A+G>T nc %; cds:ADARc_SW-A-Y MC2%; cds:ADARh_W-A-S T>C %; cds:2Gen1_-C-C C>T at MC1%; g:ADAR_2Gen1_-T-T A>T+T>A %; cds:AIDd_WR-C-Y C>A cds %; nc:A3G_C-C-C>T+G>A nc %; cds:A3Gc_C-C-GW C>T motif %; cds:ADAR_3Gen1_-A-AT Ti %; cds:3Gen3_CT-C-MC3%; cds:4Gen3_CT-C-C C>T at MC1%; cds:3Gen2_T-C-C MC1%; cds:A3G_C-C-G>T %; cds:3Gen1_-C-CA Ti %; cds:3Gen1_-C-TG G non-syn %; cds:3Gen2_A-C-C non-syn %; g:2Gen1_-C-T C>G+G>C g %; cds:All A non-syn %; cds:A3Gi_SG-C-G MC2%; cds:Primary Deaminase %; cds:4Gen3_TT-C-T %; g:A3Bj_RT-C-G C>T+G>A g %; cds:3Gen2_T-C-C MC3%; cds:4Gen3_TT-C-C %; cds:3Gen1_-C-CA Ti C:G %; cds:A1_-C-A G>A at MC3 cds %; cds:A3Gb_-C-G G>A at MC2 motif %; cds:3Gen3_CT-C-G non-syn %; cds:3Gen2_G-C-T C:G %; cds:A3Ge_SC-C-GS %; cds:3Gen3_TG-C-G>A %; g:C>A+G>T %; cds:4Gen3_CA-C-C %; cds:AIDd_WR-C-Y G>C %; cds:All G %; cds:3Gen3_TT-C-C>A at MC1 motif %; g:AIDh_WR-C-T C>A+G>T g %; g:4Gen3_GG-C-G C>T+G>A g %; cds:3Gen2_G-C-T C>A motif %; nc:ADARc_SW-A-Y A>G+T>C nc %; g:3Gen2_A-C-C C>A+G>T g %; cds:A3B_T-C-W Ti %; g:3Gen3_GA-C-C>A+G>T g %; cds:3Gen3_CT-C-C>T at MC1 motif %; cds:ADAR_3Gen1_-A-CC A>G cds %; cds:3Gen1_-C-TC C>T cds %; cds:4Gen3_CA-C-C MC1%; cds:3Gen2_G-C-T %; nc:2Gen2_A-C-C>T+G>A nc %; cds:3Gen2_A-C-C MC2%; cds:A3F_T-C-C>A %; cds:CDS Variants; cds:ADAR_3Gen3_CA-A-Ti %; cds:3Gen3_GG-C-non-syn %; cds:ADARb_W-A-Y MC2%; g:ADAR_W-A-A>G+T>C %; cds:3Gen3_AT-C-C:G %; cds:2Gen1_-C-C G>T at MC1%; cds:A3_G_C-C-MC3%; cds:3Gen2_C-C-C MC3%; cds:A3B_T-C-W G>A motif %; cds:A3F_T-C-G>C %; cds:ADAR_2Gen2_G-T-MC2%; cds:3Gen1_-C-AG G Ti/Tv %; cds:A3Bj_RT-C-G Ti %; nc:ADARb_W-A-Y A>G+T>C nc %; cds:ADAR_2Gen2_T-T-%; g:2Gen1_-C-T %; cds:4Gen3_AC-C-T Ti/Tv %; cds:A3Gi_SG-C-G non-syn %; cds:A3Bf_ST-C-G Ti %; g:ADARk_CW-A-A>G+T>C g %; cds:3Gen1_-C-GC MC2%; g:3Gen3_CA-C-C>T+G>A g %; cds:2Gen2_A-C-MC3%; variants in VCF; cds:4Gen3_AG-C-T MC1 non-syn %; g:3Gen2_T-C-G C>T+G>A g %; cds:A3Gn_YYC-C-S C>T at MC3 cds %; cds:ADAR_3Gen1_-A-CA %; cds:4Gen3_TA-C-C non-syn %; cds:All C Ti/Tv %; cds:ADARc_SW-A-Y, and related metrics thereto; or
(f) the cancer is skin cancer and the plurality of metrics comprises least or about 5 metrics selected from cds:4Gen3_AG-C-T MC1 non-syn %; cds:3Gen1_-C-CG G>A at MC3%; cds:4Gen3_AC-C-T Ti/Tv %; g:C>G+G>C %; cds:A3B_T-C-W MC3 non-syn %; cds:All A non-syn %; cds:3Gen3_AG-C-MC2%; cds:A3B_T-C-W MC1%; cds:ADAR_3Gen2_C-A-C T>G at MC3 cds %; cds:3Gen1_-C-TC C>T at MC3%; cds:4Gen3_GC-C-C C>T at MC2%; cds:All C Ti/Tv %; cds:A3Bj_RT-C-G Ti %; cds:AIDh_WR-C-T G>A at MC2 cds %; cds:4Gen3_TT-C-C %; cds:3Gen1_-C-CC C>T at MC1 motif %; cds:ADAR_2Gen2_T-T-%; cds:3Gen2_T-C-C MC1%; cds:All G %; cds:ADAR_W-A-A>G at MC3%; cds:A3G_C-C-MC3%; cds:Other MC3 C %; g:3Gen2_A-C-C C>A+G>T g %; cds:ADARc_SW-A-Y MC2%; cds:3Gen1_-C-CA Ti C:G %; cds:3Gen1_-C-TC C>T cds %; cds:3Gen2_C-C-C MC3%; cds:3Gen3_CT-C-C>T at MC1 motif %; g:ADAR_4Gen3_AG-A-G A>C+T>G %; cds:3Gen3_CT-C-G non-syn %; cds:3Gen2_A-C-C non-syn %; cds:2Gen2_A-C-MC3%; cds:3Gen2_A-C-C MC2%; g:3Gen1_-C-TC C>T+G>A g %; cds:3Gen2_T-C-T G>A at MC2%; cds:2Gen1_-C-C C>T at MC1%; cds:AIDb_WR-C-G G non-syn %; cds:A3Gb_-C-G MC1%; cds:2Gen1_-C-C C>A %; cds:A3Ge_SC-C-GS %; g:ADARn_-A-WA A>G+T>C %; g:ADAR_W-A-A>G+T>C %; g:ADAR_2Gen2_G-T-A>T+T>A %; g:AIDh_WR-C-T C>A+G>T g %; cds:4Gen3_TG-C-T Ti C:G %; cds:3Gen2_G-C-T C:G %; cds:3Gen2_T-C-C MC3%; nc:ADARb_W-A-Y %; cds:ADAR_3Gen2_G-A-C non-syn %; cds:ADAR_3Gen1_-A-AT Ti %; g:ADARk_CW-A-A>G+T>C g %; cds:3Gen1_-C-GC MC2%; cds:4Gen3_TA-C-C non-syn %; g:3Gen3_CA-C-C>T+G>A g %; cds:3Gen1_-C-AG G Ti/Tv %; cds:AIDc_WR-C-GS %; cds:A3Gn_YYC-C-S C>T at MC3 cds %; cds:2Gen1_-C-C MC2%; cds:3Gen3_GG-C-non-syn %; g:2Gen1_-C-T C>G+G>C g %; cds:A1_-C-A G>A at MC3 cds %; cds:A3G C-C-C>T at MC1%; nc:ADARc_SW-A-Y A>G+T>C nc %; cds:ADAR_W-A-T>C at MC2%; cds:A3Go_TC-C-G MC1 non-syn %; cds:3Gen3_AT-C-C:G %; cds:ADARh_W-A-S T>C %; cds:A3_G_C-C-G>T %; cds:ADARf_SW-A-MC2%; cds:ADAR_W-A-non-syn %; cds:ADARp_-A-WT T>A motif %; cds:4Gen3_AG-C-T G>A at MC1 motif %; cds:ADAR_3Gen1_-A-CA %; cds:3Gen2_C-C-T MC3%; cds:3Gen1_-C-CT C>T at MC2 cds %; cds:A3B_T-C-W Ti %; g:2Gen1_-C-T %; cds:AIDc_WR-C-GS MC3%; cds:AIDe_WR-C-GW Hits; cds:AIDd_WR-C-Y C>A cds %; cds:ADARb_W-A-Y MC2%; cds:A3Gc_C-C-GW C>T motif %; cds:2Gen1_-C-C G>T at MC1%; cds:3Gen1_-C-CA Ti %; cds:Other G MC3 Ti/Tv %; cds:CDS Variants; cds:ADAR_3Gen1_-A-CC A>G cds %; cds:A3Gn_YYC-C-S C>T %; cds:A3Bf_ST-C-G Ti %; cds:2Gen2_G-C-Hits; cds:AIDd_WR-C-Y %; cds:A3F_T-C-G>C %; cds:4Gen3_CT-C-C C>T at MC1%; cds:AIDd_WR-C-Y G>C %; cds:A3Gi_SG-C-G MC2%; cds:Other MC3%; nc:2Gen1_-C-T C>A+G>T nc %; cds:3Gen2_G-C-T %; g:3Gen2_T-C-G C>T+G>A g %; cds:ADARc_SW-A-Y T>C cds %, and related metrics thereto.

7. The method of claim 1, wherein the biological sample has been obtained from the tissue type affected by the cancer.

8. The method of claim 7, wherein the biological sample contains ovarian, breast, prostate, liver, colon, stomach, pancreatic, skin, thyroid, cervical, lymphoid, hematopoietic, bladder, lung, renal, rectal, uterine, and head or neck tissue or cells.

9. A method for treating a subject with cancer, comprising exposing to the subject a cancer therapy on the basis of a determination that the cancer or tumour is likely to progress or recur according to the method of claim 1.

10. A method of treating a cancer in a subject, the method comprising:
(i) performing the method according to claim 1;
(ii) determining that the cancer is likely to progress or recur; and
(iii) exposing the subject to a cancer therapy.

11. The method of claim 9, wherein the therapy is selected from among radiotherapy, surgery, chemotherapy, hormone therapy, immunotherapy and targeted therapy.

12. A system for generating a progression indicator for use in assessing the likelihood of cancer progression or recurrence in a subject, the system including one or more electronic processing devices that:
a) obtain subject data indicative of a sequence of a nucleic acid molecule from the subject;
b) analyze the subject data to identify single nucleotide variations (SNVs) within the nucleic acid molecule;
c) determine a plurality of metrics using the identified SNVs, the plurality of metrics including 5 or more metrics selected from the metrics set forth in Table D and metrics related to the metrics set forth in Table D;
d) apply the plurality of metrics to at least one computational model to determine a progression indicator indicative of a likelihood of progression or recurrence of a cancer, the at least one computational model embodying a relationship between a likelihood of progression or recurrence of a cancer and the plurality of metrics and being derived by applying machine learning to a plurality of reference metrics obtained from reference subjects having a known progression or recurrence of a cancer.

13. The system of claim 12, wherein the plurality of metrics comprises at least 10, 15, 20, 35, 30, 40, 45 or 50 metrics selected from selected from the metrics set forth in Table D and metrics related to the metrics set forth in Table D.

14. The system of claim 12, wherein the cancer is selected from among adrenal cancer, breast cancer, brain cancer, prostate cancer, liver cancer, colon cancer, stomach cancer, pancreatic cancer, skin cancer, thyroid, cervical cancer, lymphoid cancer, hematopoietic cancer, bladder cancer, lung cancer, renal cancer, rectal cancer, ovarian cancer, uterine cancer, head and neck cancer, mesothelioma and sarcoma.

15. The system of claim 12, wherein:
a) the cancer is mesothelioma and the plurality of metrics comprises least or about 5 metrics selected from cds:A3Bf_ST-C-G Ti %; g:3Gen2_T-C-G C>T+G>A g %; cds:2Gen1_-C-C C>T at MC1%; cds:All C Ti/Tv %; g:3Gen3_CA-C-C>T+G>A g %; cds:3Gen2_C-C-C MC3%; cds:A3Gn_YYC-C-S C>T %; cds:A3G_C-C-MC3%; cds:3Gen3_GG-C-non-syn %; g:3Gen2_A-C-C C>A+G>T g %; cds:4Gen3_TT-C-C %; cds:3Gen2_C-C-T MC3%; g:2Gen1_-C-T C>G+G>C g %; cds:Primary Deaminase %; cds:A3Gb_-C-G G>A at MC2 motif %; cds:4Gen3_CA-C-C %; cds:A3G_C-C-G>T %; cds:A3Gi_SG-C-G non-syn %; g:C>G+G>C %; cds:Other MC3%; cds:A3B_T-C-W G>A motif %, and related metrics thereto;
b) the cancer is adrenocortical carcinoma and the plurality of metrics comprises least or about 5 metrics selected from cds:All G total; cds:3Gen1_-C-TG G non-syn %; g:A3F_T-C-Hits; cds:3Gen3_GG-C-non-syn %; cds:3Gen1_-C-GT G>A motif %; cds:A3Bj_RT-C-G Ti %; cds:3Gen2_C-C-T MC3%; nc:A3G_C-C-C>T+G>A nc %; cds:AIDd_WR-C-Y %; cds:3Gen1_-C-TC C>T cds %; cds:A3B_T-C-W G>A motif %; g:CG total; cds:A3G_C-C-MC3%; cds:AIDb_WR-C-G G non-syn %; cds:A3G_C-C-C>T at MC1%; cds:3Gen3_TG-C-G>A %; g:3Gen3_GA-C-C>A+G>T g %; cds:3Gen2_A-C-G MC2 non-syn %; cds:3Gen3_CT-C-MC3%; cds:ADAR_2Gen2_G-T-MC2%; cds:ADAR_3Gen3_CA-A-Ti %; g:AIDh_WR-C-T C>A+G>T g %; cds:A3B_T-C-W MC3 non-syn %; cds:2Gen1_-C-C C>A %; cds:A1_-C-A G>A at MC3 cds %; cds:3Gen1_-C-CA Ti C:G %; cds:ADAR_W-A-non-syn %; cds:3Gen1_-C-CA Ti %; cds:All G %; g:3Gen2_T-C-G C>T+G>A g %; cds:A3Gb_-C-G MC1%; cds:A3B_T-C-W G non-syn %; nc:2Gen2_A-C-C>T+G>A nc %; cds:A3Gi_SG-C-G non-syn %; cds:Other G MC3 Ti/Tv %; cds:A3Gb_-C-G G>A at MC2 motif %; cds:A3B_T-C-W Ti %; and g:2Gen1_-C-T %, and related metrics thereto;
c) The cancer is brain cancer and the plurality of metrics comprises least or about 5 metrics selected from g:CG total; cds:AIDd_WR-C-Y %; variants in VCF; cds:4Gen3_TA-C-C non-syn %; cds:3Gen2_C-C-T MC3%; cds:AIDd_WR-C-Y G>C %; cds:A3Gb_-C-G MC1%; g:3Gen2_T-C-G C>T+G>A g %; cds:A3B_T-C-W G non-syn %; g:3Gen3_GA-C-C>A+G>T g %; cds:2Gen2_G-C-Hits; cds:AIDc_WR-C-GS MC3%; cds:All G total; cds:All A non-syn %; cds:ADAR_2Gen2_T-T-%; cds:3Gen2_A-C-C non-syn %; g:3Gen3_CA-C-C>T+G>A g %; g:ADARk_CW-A-A>G+T>C g %; nc:ADARb_W-A-Y A>G+T>C nc %; g:2Gen1_-C-T %; cds:Other MC3 C %; g:2Gen1_-C-T C>G+G>C g %; cds:ADAR_W-A-non-syn %; g:3Gen2_A-C-C C>A+G>T g %; g:ADAR_2Gen2_G-T-A>T+T>A %; cds:A3G_C-C-C>T at MC1%; cds:3Gen1_-C-GC MC2%; cds:3Gen2_G-C-T %; cds:A3F_T-C-G>C %; g:4Gen3_GG-C-G C>T+G>A g %; cds:A3Gb_-C-G G>A at MC2 motif %; cds:ADARb_W-A-Y MC2%; cds:All G %; g:A3F_T-C-Hits; cds:3Gen2_T-C-C MC1%; cds:A3B_T-C-W Ti %; cds:ADAR_3Gen1_-A-AT Ti %; cds:ADARh_W-A-S T>C %; cds:A3Gn_YYC-C-S C>T %; cds:A3Ge_SC-C-GS %; cds:2Gen2_A-C-MC3%; cds:ADAR_2Gen2_G-T-MC2%; cds:ADAR_3Gen3_CA-A-Ti %; cds:Primary Deaminase %; g:C>G+G>C %; cds:A3Bf_ST-C-G Ti %; cds:3Gen3_CT-C-MC3%; cds:A3Gi_SG-C-G non-syn %; cds:Other MC3%; cds:ADAR_3Gen1_-A-CA %; cds:A3F_T-C-C>A %; cds:2Gen1_-C-C C>T at MC1%; cds:A3Gc_C-C-GW C>T motif %; cds:AIDc_WR-C-GS %; g:ADAR_2Gen1_-T-T A>T+T>A %; cds:A3B_T-C-W MC1%; cds:ADAR_3Gen2_G-A-C non-syn %; cds:2Gen1_-C-C C>A %; cds:3Gen1_-C-GT G>A motif %; cds:A3Bj_RT-C-G Ti %; g:3Gen1_-C-TC C>T+G>A g %; g:C>A+G>T %; cds:3Gen2_A-C-C MC2%; cds:2Gen1_-C-C MC2%; g:3Gen2_G-C-T %; g:A3Bj_RT-C-G C>T+G>A g %; g:ADAR_W-A-A>G+T>C %; cds:3Gen3_AT-C-C:G %; cds:3Gen1_-C-TG G non-syn %; cds:Other G MC3 Ti/Tv %; cds:A3Gb_-C-G G>A MC2 Hits; cds:3Gen1_-C-TC C>T cds %; cds:2Gen1_-C-T MC3 non-syn %; cds:AIDb_WR-C-G G non-syn %; g:AIDc_WR-C-GS Hits; cds:3Gen2_T-C-C MC3%; cds:3Gen2_T-C-G Ti/Tv %; cds:A1_-C-A G>A at MC3 cds %; nc:A3G_C-C-C>T+G>A nc %; nc:2Gen2_A-C-C>T+G>A nc %; cds:3Gen3_TG-C-G Ti/Tv %; cds:3Gen1_-C-CA Ti %; cds:3Gen3_TG-C-G>A %; cds:3Gen3_CT-C-G non-syn %; cds:All C Ti/Tv %; cds:A3G_C-C-MC3%; cds:ADARc_SW-A-Y MC2%; and cds:3Gen3_GG-C-non-syn %, and related metrics thereto;
d) the cancer is sarcoma and the plurality of metrics comprises least or about 5 metrics selected from cds:Other MC3 C %; nc:ADARb_W-A-Y A>G+T>C nc %; cds:4Gen3_TT-C-T %; g:ADARk_CW-A-A>G+T>C g %; g:ADARn_-A-WA A>G+T>C %; cds:A3G_C-C-G>T %; cds:A3Gb_-C-G MC1%; nc:ADARb_W-A-Y %; cds:A3Ge_SC-C-GS %; cds:Primary Deaminase %; cds:ADAR_2Gen2_G-T-MC2%; g:4Gen3_GG-C-G C>T+G>A g %; cds:2Gen1_-C-C MC2%; cds:3Gen1_-C-GT G>A motif %; cds:A3Gn_YYC-C-S C>T %; cds:2Gen1_-C-C C>T at MC1%; cds:A3B_T-C-W MC3 non-syn %; cds:AIDd_WR-C-Y %; g:3Gen3_CA-C-C>T+G>A g %; cds:All A non-syn %; g:2Gen1_-C-T C>G+G>C g %; cds:ADARb_W-A-Y MC2%; cds:All G %; g:A3Bj_RT-C-G C>T+G>A g %; cds:A3Gn_YYC-C-S C>T at MC3 cds %; cds:A3B_T-C-W G non-syn %; cds:A3G_C-C-MC3%; cds:All G total; cds:CDS Variants; g:CG total; g:3Gen2_T-C-G C>T+G>A g %; cds:A3B_T-C-W MC1%; cds:ADAR_3Gen3_CA-A-Ti %; cds:AIDc_WR-C-GS %, and related metrics thereto;
e) the cancer is lung cancer and the plurality of metrics comprises least or about 5 metrics selected from cds:3Gen1_-C-CC C>T at MC1 motif %; cds:3Gen1_-C-CT C>T at MC2 cds %; cds:ADARp-A-WT A>G at MC2 cds %; cds:Other MC3 C %; cds:Other MC3%; cds:A3Gb_-C-G MC1%; g:3Gen1_-C-TC C>T+G>A g %; cds:ADAR_W-A-A>G at MC3%; cds:ADAR_W-A-non-syn %; cds:ADAR_3Gen3_AC-A-A>G cds %; cds:2Gen1_-C-C C>A %; cds:ADARf_SW-A-MC2%; g:ADAR_2Gen2_G-T-A>T+T>A %; cds:4Gen3_GC-C-A %; cds:A3Go_TC-C-G MC1 non-syn %; g:3Gen2_G-C-T %; cds:A3G_C-C-C>T at MC1%; cds:AIDc_WR-C-GS MC3%; cds:3Gen1_-C-GT G>A motif %; nc:2Gen1_-C-T C>A+G>T nc %; cds:ADARc_SW-A-Y MC2%; cds:ADARh_W-A-S T>C %; cds:2Gen1_-C-C C>T at MC1%; g:ADAR_2Gen1_-T-T A>T+T>A %; cds:AIDd_WR-C-Y C>A cds %; nc:A3G_C-C-C>T+G>A nc %; cds:A3Gc_C-C-GW C>T motif %; cds:ADAR_3Gen1_-A-AT Ti %; cds:3Gen3_CT-C-MC3%; cds:4Gen3_CT-C-C C>T at MC1%; cds:3Gen2_T-C-C MC1%; cds:A3G_C-C-G>T %; cds:3Gen1_-C-CA Ti %; cds:3Gen1_-C-TG G non-syn %; cds:3Gen2_A-C-C non-syn %; g:2Gen1_-C-T C>G+G>C g %; cds:All A non-syn %; cds:A3Gi_SG-C-G MC2%; cds:Primary Deaminase %; cds:4Gen3_TT-C-T %; g:A3Bj_RT-C-G C>T+G>A g %; cds:3Gen2_T-C-C MC3%; cds:4Gen3_TT-C-C %; cds:3Gen1_-C-CA Ti C:G %; cds:A1_-C-A G>A at MC3 cds %; cds:A3Gb_-C-G G>A at MC2 motif %; cds:3Gen3_CT-C-G non-syn %; cds:3Gen2_G-C-T C:G %; cds:A3Ge_SC-C-GS %; cds:3Gen3_TG-C-G>A %; g:C>A+G>T %; cds:4Gen3_CA-C-C %; cds:AIDd_WR-C-Y G>C %; cds:All G %; cds:3Gen3_TT-C-C>A at MC1 motif %; g:AIDh_WR-C-T C>A+G>T g %; g:4Gen3_GG-C-G C>T+G>A g %; cds:3Gen2_G-C-T C>A motif %; nc:ADARc_SW-A-Y A>G+T>C nc %; g:3Gen2_A-C-C C>A+G>T g %; cds:A3B_T-C-W Ti %; g:3Gen3_GA-C-C>A+G>T g %; cds:3Gen3_CT-C-C>T at MC1 motif %; cds:ADAR_3Gen1_-A-CC A>G cds %; cds:3Gen1_-C-TC C>T cds %; cds:4Gen3_CA-C-C MC1%; cds:3Gen2_G-C-T %; nc:2Gen2_A-C-C>T+G>A nc %; cds:3Gen2_A-C-C MC2%; cds:A3F_T-C-C>A %; cds:CDS Variants; cds:ADAR_3Gen3_CA-A-Ti %; cds:3Gen3_GG-C-non-syn %; cds:ADARb_W-A-Y MC2%; g:ADAR_W-A-A>G+T>C %; cds:3Gen3_AT-C-C:G %; cds:2Gen1_-C-C G>T at MC1%; cds:A3G_C-C-MC3%; cds:3Gen2_C-C-C MC3%; cds:A3B_T-C-W G>A motif %; cds:A3F_T-C-G>C %; cds:ADAR_2Gen2_G-T-MC2%; cds:3Gen1_-C-AG G Ti/Tv %; cds:A3Bj_RT-C-G Ti %; nc:ADARb_W-A-Y A>G+T>C nc %; cds:ADAR_2Gen2_T-T-%; g:2Gen1_-C-T %; cds:4Gen3_AC-C-T Ti/Tv %; cds:A3Gi_SG-C-G non-syn %; cds:A3Bf_ST-C-G Ti %; g:ADARk_CW-A-A>G+T>C g %; cds:3Gen1_-C-GC MC2%; g:3Gen3_CA-C-C>T+G>A g %; cds:2Gen2_A-C-MC3%; variants in VCF; cds:4Gen3_AG-C-T MC1 non-syn %; g:3Gen2_T-C-G C>T+G>A g %; cds:A3Gn_YYC-C-S C>T at MC3 cds %; cds:ADAR_3Gen1_-A-CA %; cds:4Gen3_TA-C-C non-syn %; cds:All C Ti/Tv %; cds:ADARc_SW-A-Y, and related metrics thereto; or
f) the cancer is skin cancer and the plurality of metrics comprises least or about 5 metrics selected from cds:4Gen3_AG-C-T MC1 non-syn %; cds:3Gen1_-C-CG G>A at MC3%; cds:4Gen3_AC-C-T Ti/Tv %; g:C>G+G>C %; cds:A3B_T-C-W MC3 non-syn %; cds:All A non-syn %; cds:3Gen3_AG-C-MC2%; cds:A3B_T-C-W MC1%; cds:ADAR_3Gen2_C-A-C T>G at MC3 cds %; cds:3Gen1_-C-TC C>T at MC3%; cds:4Gen3_GC-C-C C>T at MC2%; cds:All C Ti/Tv %; cds:A3Bj_RT-C-G Ti %; cds:AIDh_WR-C-T G>A at MC2 cds %; cds:4Gen3_TT-C-C %; cds:3Gen1_-C-CC C>T at MC1 motif %; cds:ADAR_2Gen2_T-T-%; cds:3Gen2_T-C-C MC1%; cds:All G %; cds:ADAR_W-A-A>G at MC3%; cds:A3G_C-C-MC3%; cds:Other MC3 C %; g:3Gen2_A-C-C C>A+G>T g %; cds:ADARc_SW-A-Y MC2%; cds:3Gen1_-C-CA Ti C:G %; cds:3Gen1_-C-TC C>T cds %; cds:3Gen2_C-C-C MC3%; cds:3Gen3_CT-C-C>T at MC1 motif %; g:ADAR_4Gen3_AG-A-G A>C+T>G %; cds:3Gen3_CT-C-G non-syn %; cds:3Gen2_A-C-C non-syn %; cds:2Gen2_A-C-MC3%; cds:3Gen2_A-C-C MC2%; g:3Gen1_-C-TC C>T+G>A g %; cds:3Gen2_T-C-T G>A at MC2%; cds:2Gen1_-C-C C>T at MC1%; cds:AIDb_WR-C-G G non-syn %; cds:A3Gb_-C-G MC1%; cds:2Gen1_-C-C C>A %; cds:A3Ge_SC-C-GS %; g:ADARn_-A-WA A>G+T>C %; g:ADAR_W-A-A>G+T>C %; g:ADAR_2Gen2_G-T-A>T+T>A %; g:AIDh_WR-C-T C>A+G>T g %; cds:4Gen3_TG-C-T Ti C:G %; cds:3Gen2_G-C-T C:G %; cds:3Gen2_T-C-C MC3%; nc:ADARb_W-A-Y %; cds:ADAR_3Gen2_G-A-C non-syn %; cds:ADAR_3Gen1_-A-AT Ti %; g:ADARk_CW-A-A>G+T>C g %; cds:3Gen1_-C-GC MC2%; cds:4Gen3_TA-C-C non-syn %; g:3Gen3_CA-C-C>T+G>A g %; cds:3Gen1_-C-AG G Ti/Tv %; cds:AIDc_WR-C-GS %; cds:A3Gn_YYC-C-S C>T at MC3 cds %; cds:2Gen1_-C-C MC2%; cds:3Gen3_GG-C-non-syn %; g:2Gen1_-C-T C>G+G>C g %; cds:A1_-C-A G>A at MC3 cds %; cds:A3G C-C-C>T at MC1%; nc:ADARc_SW-A-Y A>G+T>C nc %; cds:ADAR_W-A-T>C at MC2%; cds:A3Go_TC-C-G MC1 non-syn %; cds:3Gen3_AT-C-C:G %; cds:ADARh_W-A-S T>C %; cds:A3G_C-C-G>T %; cds:ADARf_SW-A-MC2%; cds:ADAR_W-A-non-syn %; cds:ADARp_-A-WT T>A motif %; cds:4Gen3_AG-C-T G>A at MC1 motif %; cds:ADAR_3Gen1_-A-CA %; cds:3Gen2_C-C-T MC3%; cds:3Gen1_-C-CT C>T at MC2 cds %; cds:A3B_T-C-W Ti %; g:2Gen1_-C-T %; cds:AIDc_WR-C-GS MC3%; cds:AIDe_WR-C-GW Hits; cds:AIDd_WR-C-Y C>A cds %; cds:ADARb_W-A-Y MC2%; cds:A3Gc_C-C-GW C>T motif %; cds:2Gen1_-C-C G>T at MC1%; cds:3Gen1_-C-CA Ti %; cds:Other G MC3 Ti/Tv %; cds:CDS Variants; cds:ADAR_3Gen1_-A-CC A>G cds %; cds:A3Gn_YYC-C-S C>T %; cds:A3Bf_ST-C-G Ti %; cds:2Gen2_G-C-Hits; cds:AIDd_WR-C-Y %; cds:A3F_T-C-G>C %; cds:4Gen3_CT-C-C C>T at MC1%; cds:AIDd_WR-C-Y G>C %; cds:A3Gi_SG-C-G MC2%; cds:Other MC3%; nc:2Gen1_-C-T C>A+G>T nc %; cds:3Gen2_G-C-T %; g:3Gen2_T-C-G C>T+G>A g %; cds:ADARc_SW-A-Y T>C cds %, and related metrics thereto.

16. The system of claim 12, wherein the at least one computational model includes a decision tree.

17. The system of claim 12, wherein the at least one computational model includes a plurality of decision trees, and wherein the therapy indicator is generated by aggregating results from the plurality of decision trees.

18. A system for use in calculating at least one computational model, the at least one computational model being used for generating a progression indicator for use in assessing likelihood of cancer progression or recurrence in a subject, the system including one or more electronic processing devices that:
a) for each of a plurality of reference subjects:
i) obtain reference subject data indicative of:
(1) a sequence of a nucleic acid molecule from the reference subject; and,
(2) progression or recurrence of cancer;
ii) analyze the reference subject data to identify single nucleotide variations (SNVs) within the nucleic acid molecule;
iii) determine a plurality of metrics using the identified SNVs, the plurality of metrics including 5 or more metrics selected from the metrics set forth in Table D and metrics related to the metrics set forth in Table D; and,
b) use the plurality of reference metrics and known progression or recurrence of cancer of reference subjects to train at least one computational model, the at least one computational model embodying a relationship between progression or recurrence of cancer and the plurality of metrics.

19. The system of claim 18, wherein the plurality of metrics comprises at least 10, 15, 20, 35, 30, 40, 45 or 50 metrics selected from selected from the metrics set forth in Table D and metrics related to the metrics set forth in Table D.

20. The system of claim 18, wherein the cancer is selected from among adrenal cancer, breast cancer, brain cancer, prostate cancer, liver cancer, colon cancer, stomach cancer, pancreatic cancer, skin cancer, thyroid, cervical cancer, lymphoid cancer, hematopoietic cancer, bladder cancer, lung cancer, renal cancer, rectal cancer, ovarian cancer, uterine cancer, head and neck cancer, mesothelioma and sarcoma.

21. The system of claim 18, wherein:
a) the cancer is mesothelioma and the plurality of metrics comprises least or about 5 metrics selected from cds:A3Bf_ST-C-G Ti %; g:3Gen2_T-C-G C>T+G>A g %; cds:2Gen1-C-C C>T at MC1%; cds:All C Ti/Tv %; g:3Gen3_CA-C-C>T+G>A g %; cds:3Gen2_C-C-C MC3%; cds:A3Gn_YYC-C-S C>T %; cds:A3G_C-C-MC3%; cds:3Gen3_GG-C-non-syn %; g:3Gen2_A-C-C C>A+G>T g %; cds:4Gen3_TT-C-C %; cds:3Gen2_C-C-T MC3%; g:2Gen1_-C-T C>G+G>C g %; cds:Primary Deaminase %; cds:A3Gb_-C-G G>A at MC2 motif %; cds:4Gen3_CA-C-C %; cds:A3G_C-C-G>T %; cds:A3Gi_SG-C-G non-syn %; g:C>G+G>C %; cds:Other MC3%; cds:A3B_T-C-W G>A motif %, and related metrics thereto;
b) the cancer is adrenocortical carcinoma and the plurality of metrics comprises least or about 5 metrics selected from cds:All G total; cds:3Gen1_-C-TG G non-syn %; g:A3F_T-C-Hits; cds:3Gen3_GG-C-non-syn %; cds:3Gen1_-C-GT G>A motif %; cds:A3Bj_RT-C-G Ti %; cds:3Gen2_C-C-T MC3%; nc:A3G_C-C-C>T+G>A nc %; cds:AIDd_WR-C-Y %; cds:3Gen1_-C-TC C>T cds %; cds:A3B_T-C-W G>A motif %; g:CG total; cds:A3G_C-C-MC3%; cds:AIDb_WR-C-G G non-syn %; cds:A3G_C-C-C>T at MC1%; cds:3Gen3_TG-C-G>A %; g:3Gen3_GA-C-C>A+G>T g %; cds:3Gen2_A-C-G MC2 non-syn %; cds:3Gen3_CT-C-MC3%; cds:ADAR_2Gen2_G-T-MC2%; cds:ADAR_3Gen3_CA-A-Ti %; g:AIDh_WR-C-T C>A+G>T g %; cds:A3B_T-C-W MC3 non-syn %; cds:2Gen1_-C-C C>A %; cds:A1-C-A G>A at MC3 cds %; cds:3Gen1_-C-CA Ti C:G %; cds:ADAR_W-A-non-syn %; cds:3Gen1_-C-CA Ti %; cds:All G %; g:3Gen2_T-C-G C>T+G>A g %; cds:A3Gb_-C-G MC1%; cds:A3B_T-C-W G non-syn %; nc:2Gen2_A-C-C>T+G>A nc %; cds:A3Gi_SG-C-G non-syn %; cds:Other G MC3 Ti/Tv %; cds:A3Gb_-C-G G>A at MC2 motif %; cds:A3B_T-C-W Ti %; and g:2Gen1_-C-T %, and related metrics thereto;
c) The cancer is brain cancer and the plurality of metrics comprises least or about 5 metrics selected from g:CG total; cds:AIDd_WR-C-Y %; variants in VCF; cds:4Gen3_TA-C-C non-syn %; cds:3Gen2_C-C-T MC3%; cds:AIDd_WR-C-Y G>C %; cds:A3Gb_-C-G MC1%; g:3Gen2_T-C-G C>T+G>A g %; cds:A3B_T-C-W G non-syn %; g:3Gen3_GA-C-C>A+G>T g %; cds:2Gen2_G-C-Hits; cds:AIDc_WR-C-GS MC3%; cds:All G total; cds:All A non-syn %; cds:ADAR_2Gen2_T-T-%; cds:3Gen2_A-C-C non-syn %; g:3Gen3_CA-C-C>T+G>A g %; g:ADARk_CW-A-A>G+T>C g %; nc:ADARb_W-A-Y A>G+T>C nc %; g:2Gen1_-C-T %; cds:Other MC3 C %; g:2Gen1_-C-T C>G+G>C g %; cds:ADAR_W-A-non-syn %; g:3Gen2_A-C-C C>A+G>T g %; g:ADAR_2Gen2_G-T-A>T+T>A %; cds:A3G_C-C-C>T at MC1%; cds:3Gen1_-C-GC MC2%; cds:3Gen2_G-C-T %; cds:A3F_T-C-G>C %; g:4Gen3_GG-C-G C>T+G>A g %; cds:A3Gb_-C-G G>A at MC2 motif %; cds:ADARb_W-A-Y MC2%; cds:All G %; g:A3F_T-C-Hits; cds:3Gen2_T-C-C MC1%; cds:A3B_T-C-W Ti %; cds:ADAR_3Gen1_-A-AT Ti %; cds:ADARh_W-A-S T>C %; cds:A3Gn_YYC-C-S C>T %; cds:A3Ge_SC-C-GS %; cds:2Gen2_A-C-MC3%; cds:ADAR_2Gen2_G-T-MC2%; cds:ADAR_3Gen3_CA-A-Ti %; cds:Primary Deaminase %; g:C>G+G>C %; cds:A3Bf_ST-C-G Ti %; cds:3Gen3_CT-C-MC3%; cds:A3Gi_SG-C-G non-syn %; cds:Other MC3%; cds:ADAR_3Gen1_-A-CA %; cds:A3F_T-C-C>A %; cds:2Gen1_-C-C C>T at MC1%; cds:A3Gc_C-C-GW C>T motif %; cds:AIDc_WR-C-GS %; g:ADAR_2Gen1_-T-T A>T+T>A %; cds:A3B_T-C-W MC1%; cds:ADAR_3Gen2_G-A-C non-syn %; cds:2Gen1_-C-C C>A %; cds:3Gen1_-C-GT G>A motif %; cds:A3Bj_RT-C-G Ti %; g:3Gen1_-C-TC C>T+G>A g %; g:C>A+G>T %; cds:3Gen2_A-C-C MC2%; cds:2Gen1_-C-C MC2%; g:3Gen2_G-C-T %; g:A3Bj_RT-C-G C>T+G>A g %; g:ADAR_W-A-A>G+T>C %; cds:3Gen3_AT-C-C:G %; cds:3Gen1_-C-TG G non-syn %; cds:Other G MC3 Ti/Tv %; cds:A3Gb_-C-G G>A MC2 Hits; cds:3Gen1_-C-TC C>T cds %; cds:2Gen1_-C-T MC3 non-syn %; cds:AIDb_WR-C-G G non-syn %; g:AIDc_WR-C-GS Hits; cds:3Gen2_T-C-C MC3%; cds:3Gen2_T-C-G Ti/Tv %; cds:A1_-C-A G>A at MC3 cds %; nc:A3G_C-C-C>T+G>A nc %; nc:2Gen2_A-C-C>T+G>A nc %; cds:3Gen3_TG-C-G Ti/Tv %; cds:3Gen1_-C-CA Ti %; cds:3Gen3_TG-C-G>A %; cds:3Gen3_CT-C-G non-syn %; cds:All C Ti/Tv %; cds:A3G_C-C-MC3%; cds:ADARc_SW-A-Y MC2%; and cds:3Gen3_GG-C-non-syn %, and related metrics thereto;
d) the cancer is sarcoma and the plurality of metrics comprises least or about 5 metrics selected from cds:Other MC3 C %; nc:ADARb_W-A-Y A>G+T>C nc %; cds:4Gen3_TT-C-T %; g:ADARk_CW-A-A>G+T>C g %; g:ADARn_-A-WA A>G+T>C %; cds:A3G_C-C-G>T %; cds:A3Gb_-C-G MC1%; nc:ADARb_W-A-Y %; cds:A3Ge_SC-C-GS %; cds:Primary Deaminase %; cds:ADAR_2Gen2_G-T-MC2%; g:4Gen3_GG-C-G C>T+G>A g %; cds:2Gen1_-C-C MC2%; cds:3Gen1_-C-GT G>A motif %; cds:A3Gn_YYC-C-S C>T %; cds:2Gen1_-C-C C>T at MC1%; cds:A3B_T-C-W MC3 non-syn %; cds:AIDd_WR-C-Y %; g:3Gen3_CA-C-C>T+G>A g %; cds:All A non-syn %; g:2Gen1_-C-T C>G+G>C g %; cds:ADARb_W-A-Y MC2%; cds:All G %; g:A3Bj_RT-C-G C>T+G>A g %; cds:A3Gn_YYC-C-S C>T at MC3 cds %; cds:A3B_T-C-W G non-syn %; cds:A3G_C-C-MC3%; cds:All G total; cds:CDS Variants; g:CG total; g:3Gen2_T-C-G C>T+G>A g %; cds:A3B_T-C-W MC1%; cds:ADAR_3Gen3_CA-A-Ti %; cds:AIDc_WR-C-GS %, and related metrics thereto;
e) the cancer is lung cancer and the plurality of metrics comprises least or about 5 metrics selected from cds:3Gen1_-C-CC C>T at MC1 motif %; cds:3Gen1_-C-CT C>T at MC2 cds %; cds:ADARp-A-WT A>G at MC2 cds %; cds:Other MC3 C %; cds:Other MC3%; cds:A3Gb_-C-G MC1%; g:3Gen1_-C-TC C>T+G>A g %; cds:ADAR_W-A-A>G at MC3%; cds:ADAR_W-A-non-syn %; cds:ADAR_3Gen3_AC-A-A>G cds %; cds:2Gen1_-C-C C>A %; cds:ADARf_SW-A-MC2%; g:ADAR_2Gen2_G-T-A>T+T>A %; cds:4Gen3_GC-C-A %; cds:A3Go_TC-C-G MC1 non-syn %; g:3Gen2_G-C-T %; cds:A3G_C-C-C>T at MC1%; cds:AIDc_WR-C-GS MC3%; cds:3Gen1_-C-GT G>A motif %; nc:2Gen1_-C-T C>A+G>T nc %; cds:ADARc_SW-A-Y MC2%; cds:ADARh_W-A-S T>C %; cds:2Gen1_-C-C C>T at MC1%; g:ADAR_2Gen1_-T-T A>T+T>A %; cds:AIDd_WR-C-Y C>A cds %; nc:A3G_C-C-C>T+G>A nc %; cds:A3Gc_C-C-GW C>T motif %; cds:ADAR_3Gen1_-A-AT Ti %; cds:3Gen3_CT-C-MC3%; cds:4Gen3_CT-C-C C>T at MC1%; cds:3Gen2_T-C-C MC1%; cds:A3G_C-C-G>T %; cds:3Gen1_-C-CA Ti %; cds:3Gen1_-C-TG G non-syn %; cds:3Gen2_A-C-C non-syn %; g:2Gen1_-C-T C>G+G>C g %; cds:All A non-syn %; cds:A3Gi_SG-C-G MC2%; cds:Primary Deaminase %; cds:4Gen3_TT-C-T %; g:A3Bj_RT-C-G C>T+G>A g %; cds:3Gen2_T-C-C MC3%; cds:4Gen3_TT-C-C %; cds:3Gen1_-C-CA Ti C:G %; cds:A1_-C-A G>A at MC3 cds %; cds:A3Gb_-C-G G>A at MC2 motif %; cds:3Gen3_CT-C-G non-syn %; cds:3Gen2_G-C-T C:G %; cds:A3Ge_SC-C-GS %; cds:3Gen3_TG-C-G>A %; g:C>A+G>T %; cds:4Gen3_CA-C-C %; cds:AIDd_WR-C-Y G>C %; cds:All G %; cds:3Gen3_TT-C-C>A at MC1 motif %; g:AIDh_WR-C-T C>A+G>T g %; g:4Gen3_GG-C-G C>T+G>A g %; cds:3Gen2_G-C-T C>A motif %; nc:ADARc_SW-A-Y A>G+T>C nc %; g:3Gen2_A-C-C C>A+G>T g %; cds:A3B_T-C-W Ti %; g:3Gen3_GA-C-C>A+G>T g %; cds:3Gen3_CT-C-C>T at MC1 motif %; cds:ADAR_3Gen1_-A-CC A>G cds %; cds:3Gen1_-C-TC C>T cds %; cds:4Gen3_CA-C-C MC1%; cds:3Gen2_G-C-T %; nc:2Gen2_A-C-C>T+G>A nc %; cds:3Gen2_A-C-C MC2%; cds:A3F_T-C-C>A %; cds:CDS Variants; cds:ADAR_3Gen3_CA-A-Ti %; cds:3Gen3_GG-C-non-syn %; cds:ADARb_W-A-Y MC2%; g:ADAR_W-A-A>G+T>C %; cds:3Gen3_AT-C-C:G %; cds:2Gen1_-C-C G>T at MC1%; cds:A3G_C-C-MC3%; cds:3Gen2_C-C-C MC3%; cds:A3B_T-C-W G>A motif %; cds:A3F_T-C-G>C %; cds:ADAR_2Gen2_G-T-MC2%; cds:3Gen1_-C-AG G Ti/Tv %; cds:A3Bj_RT-C-G Ti %; nc:ADARb_W-A-Y A>G+T>C nc %; cds:ADAR_2Gen2_T-T-%; g:2Gen1_-C-T %; cds:4Gen3_AC-C-T Ti/Tv %; cds:A3Gi_SG-C-G non-syn %; cds:A3Bf_ST-C-G Ti %; g:ADARk_CW-A-A>G+T>C g %; cds:3Gen1_-C-GC MC2%; g:3Gen3_CA-C-C>T+G>A g %; cds:2Gen2_A-C-MC3%; variants in VCF; cds:4Gen3_AG-C-T MC1 non-syn %; g:3Gen2_T-C-G C>T+G>A g %; cds:A3Gn_YYC-C-S C>T at MC3 cds %; cds:ADAR_3Gen1_-A-CA %; cds:4Gen3_TA-C-C non-syn %; cds:All C Ti/Tv %; cds:ADARc_SW-A-Y, and related metrics thereto; or
f) the cancer is skin cancer and the plurality of metrics comprises least or about 5 metrics selected from cds:4Gen3_AG-C-T MC1 non-syn %; cds:3Gen1_-C-CG G>A at MC3%; cds:4Gen3_AC-C-T Ti/Tv %; g:C>G+G>C %; cds:A3B_T-C-W MC3 non-syn %; cds:All A non-syn %; cds:3Gen3_AG-C-MC2%; cds:A3B_T-C-W MC1%; cds:ADAR_3Gen2_C-A-C T>G at MC3 cds %; cds:3Gen1_-C-TC C>T at MC3%; cds:4Gen3_GC-C-C C>T at MC2%; cds:All C Ti/Tv %; cds:A3Bj_RT-C-G Ti %; cds:AIDh_WR-C-T G>A at MC2 cds %; cds:4Gen3_TT-C-C %; cds:3Gen1_-C-CC C>T at MC1 motif %; cds:ADAR_2Gen2_T-T-%; MC3%; cds:Other MC3 C %; g:3Gen2_A-C-C C>A+G>T g %; cds:ADARc_SW-A-Y MC2%; cds:3Gen1_-C-CA Ti C:G %; cds:3Gen1_-C-TC C>T cds %; cds:3Gen2_C-C-C MC3%; cds:3Gen3_CT-C-C>T at MC1 motif %; g:ADAR_4Gen3_AG-A-G A>C+T>G %; cds:3Gen3_CT-C-G non-syn %; cds:3Gen2_A-C-C non-syn %; cds:2Gen2_A-C-MC3%; cds:3Gen2_A-C-C MC2%; g:3Gen1_-C-TC C>T+G>A g %; cds:3Gen2_T-C-T G>A at MC2%; cds:2Gen1_-C-C C>T at MC1%; cds:AIDb_WR-C-G G non-syn %; cds:A3Gb_-C-G MC1%; cds:2Gen1_-C-C C>A %; cds:A3Ge_SC-C-GS %; g:ADARn_-A-WA A>G+T>C %; g:ADAR_W-A-A>G+T>C %; g:ADAR_2Gen2_G-T-A>T+T>A %; g:AIDh_WR-C-T C>A+G>T g %; cds:4Gen3_TG-C-T Ti C:G %; cds:3Gen2_G-C-T C:G %; cds:3Gen2_T-C-C MC3%; nc:ADARb_W-A-Y %; cds:ADAR_3Gen2_G-A-C non-syn %; cds:ADAR_3Gen1_-A-AT Ti %; g:ADARk_CW-A-A>G+T>C g %; cds:3Gen1_-C-GC MC2%; cds:4Gen3_TA-C-C non-syn %; g:3Gen3_CA-C-C>T+G>A g %; cds:3Gen1_-C-AG G Ti/Tv %; cds:AIDc_WR-C-GS %; cds:A3Gn_YYC-C-S C>T at MC3 cds %; cds:2Gen1_-C-C MC2%; cds:3Gen3_GG-C-non-syn %; g:2Gen1_-C-T C>G+G>C g %; cds:A1_-C-A G>A at MC3 cds %; cds:A3G_C-C-C>T at MC1%; nc:ADARc_SW-A-Y A>G+T>C nc %; cds:ADAR_W-A-T>C at MC2%; cds:A3Go_TC-C-G MC1 non-syn %; cds:3Gen3_AT-C-C:G %; cds:ADARh_W-A-S T>C %; cds:A3G_C-C-G>T %; cds:ADARf_SW-A-MC2%; cds:ADAR_W-A-non-syn %; cds:ADARp_-A-WT T>A motif %; cds:4Gen3_AG-C-T G>A at MC1 motif %; cds:ADAR_3Gen1_-A-CA %; cds:3Gen2_C-C-T MC3%; cds:3Gen1_-C-CT C>T at MC2 cds %; cds:A3B_T-C-W Ti %; g:2Gen1_-C-T %; cds:AIDc_WR-C-GS MC3%; cds:AIDe_WR-C-GW Hits; cds:AIDd_WR-C-Y C>A cds %; cds:ADARb_W-A-Y MC2%; cds:A3Gc_C-C-GW C>T motif %; cds:2Gen1_-C-C G>T at MC1%; cds:3Gen1_-C-CA Ti %; cds:Other G MC3 Ti/Tv %; cds:CDS Variants; cds:ADAR_3Gen1_-A-CC A>G cds %; cds:A3Gn_YYC-C-S C>T %; cds:A3Bf_ST-C-G Ti %; cds:2Gen2_G-C-Hits; cds:AIDd_WR-C-Y %; cds:A3F_T-C-G>C %; cds:4Gen3_CT-C-C C>T at MC1%; cds:AIDd_WR-C-Y G>C %; cds:A3Gi_SG-C-G MC2%; cds:Other MC3%; nc:2Gen1_-C-T C>A+G>T nc %; cds:3Gen2_G-C-T %; g:3Gen2_T-C-G C>T+G>A g %; cds:ADARc_SW-A-Y T>C cds %, and related metrics thereto.

22. The system of claim 18, wherein the one or more processing devices test the at least one computational model to determine a discriminatory performance of the model.

23. The system of claim 22, wherein the discriminatory performance is based on at least one of:
a) an area under a receiver operating characteristic curve;
b) an accuracy;
c) a sensitivity; and,
d) a specificity.

24. A system according to claim 22, wherein the discriminatory performance is at least 60%.

25. The system of claim 18, wherein the one or more processing devices test the at least one computational model using a reference subject data from a subset of the plurality of reference subjects.

26. The system of claim 18, wherein the one or more processing devices:
a) select a plurality of reference metrics;
b) train at least one computational model using the plurality of reference metrics;
c) test the at least one computational model to determine a discriminatory performance of the model; and,
d) if the discriminatory performance of the model falls below a threshold, at least one of:
i) selectively retrain the at least one computational model using a different plurality of reference metrics; and,
ii) train a different computational model.

27. The system of claim 18, wherein the one or more processing devices:
a) select a plurality of combinations of reference metrics;
b) train a plurality of computational models using each of the combinations;
c) test each computational model to determine a discriminatory performance of the model; and,
d) selecting the at least one computational model with the highest discriminatory performance for use in determining the progression indicator.

28. A method for generating a progression indicator for use in assessing likelihood of cancer progression or recurrence in a subject, the method including, in one or more electronic processing devices:
a) obtaining subject data indicative of a sequence of a nucleic acid molecule from the subject;
b) analyzing the subject data to identify single nucleotide variations (SNVs) within the nucleic acid molecule;
c) determining a plurality of metrics using the identified SNVs, the plurality of metrics including 5 or more metrics selected from the metrics set forth in Table D and metrics related to the metrics set forth in Table D; and,
d) applying the plurality of metrics to at least one computational model to determine a progression indicator indicative of progression or recurrence of cancer, the at least one computational model embodying a relationship between progression or recurrence of cancer and the plurality of metrics and being derived by applying machine learning to a plurality of reference metrics obtained from reference subjects having a known progression or recurrence of cancer.

29. The method of claim 28, wherein the plurality of metrics comprises at least 10, 15, 20, 35, 30, 40, 45 or 50 metrics selected from selected from the metrics set forth in Table D and metrics related to the metrics set forth in Table D.

30. The method of claim 28, wherein the cancer is selected from among adrenal cancer, breast cancer, brain cancer, prostate cancer, liver cancer, colon cancer, stomach cancer, pancreatic cancer, skin cancer, thyroid, cervical cancer, lymphoid cancer, hematopoietic cancer, bladder cancer, lung cancer, renal cancer, rectal cancer, ovarian cancer, uterine cancer, head and neck cancer, mesothelioma and sarcoma.

31. The method of claim 28, wherein:
a) the cancer is mesothelioma and the plurality of metrics comprises least or about 5 metrics selected from cds:A3Bf_ST-C-G Ti %; g:3Gen2_T-C-G C>T+G>A g %; cds:2Gen1_-C-C C>T at MC1%; cds:All C Ti/Tv %; g:3Gen3_CA-C-C>T+G>A g %; cds:3Gen2_C-C-C MC3%; cds:A3Gn_YYC-C-S C>T %; cds:A3G_C-C-MC3%; cds:3Gen3_GG-C-non-syn %; g:3Gen2_A-C-C C>A+G>T g %; cds:4Gen3_TT-C-C %; cds:3Gen2_C-C-T MC3%; g:2Gen1_-C-T C>G+G>C g %; cds:Primary Deaminase %; cds:A3Gb_-C-G G>A at MC2 motif %; cds:4Gen3_CA-C-C %; cds:A3G_C-C-G>T %; cds:A3Gi_SG-C-G non-syn %; g:C>G+G>C %; cds:Other MC3%; cds:A3B_T-C-W G>A motif %, and related metrics thereto;
b) the cancer is adrenocortical carcinoma and the plurality of metrics comprises least or about 5 metrics selected from cds:All G total; cds:3Gen1_-C-TG G non-syn %; g:A3F_T-C-Hits; cds:3Gen3_GG-C-non-syn %; cds:3Gen1_-C-GT G>A motif %; cds:A3Bj_RT-C-G Ti %; cds:3Gen2_C-C-T MC3%; nc:A3G_C-C-C>T+G>A nc %; cds:AIDd_WR-C-Y %; cds:3Gen1_-C-TC C>T cds %; cds:A3B_T-C-W G>A motif %; g:CG total; cds:A3G_C-C-MC3%; cds:AIDb_WR-C-G G non-syn %; cds:A3G_C-C-C>T at MC1%; cds:3Gen3_TG-C-G>A %; g:3Gen3_GA-C-C>A+G>T g %; cds:3Gen2_A-C-G MC2 non-syn %; cds:3Gen3_CT-C-MC3%; cds:ADAR_2Gen2_G-T-MC2%; cds:ADAR_3Gen3_CA-A-Ti %; g:AIDh_WR-C-T C>A+G>T g %; cds:A3B_T-C-W MC3 non-syn %; cds:2Gen1_-C-C C>A %; cds:A1-C-A G>A at MC3 cds %; cds:3Gen1_-C-CA Ti C:G %; cds:ADAR_W-A-non-syn %; cds:3Gen1_-C-CA Ti %; cds:All G %; g:3Gen2_T-C-G C>T+G>A g %; cds:A3Gb_-C-G MC1%; cds:A3B_T-C-W G non-syn %; nc:2Gen2_A-C-C>T+G>A nc %; cds:A3Gi_SG-C-G non-syn %; cds:Other G MC3 Ti/Tv %; cds:A3Gb_-C-G G>A at MC2 motif %; cds:A3B_T-C-W Ti %; and g:2Gen1_-C-T %, and related metrics thereto;
c) the cancer is brain cancer and the plurality of metrics comprises least or about 5 metrics selected from g:CG total; cds:AIDd_WR-C-Y %; variants in VCF; cds:4Gen3_TA-C-C non-syn %; cds:3Gen2_C-C-T MC3%; cds:AIDd_WR-C-Y G>C %; cds:A3Gb_-C-G MC1%; g:3Gen2_T-C-G C>T+G>A g %; cds:A3B_T-C-W G non-syn %; g:3Gen3_GA-C-C>A+G>T g %; cds:2Gen2_G-C-Hits; cds:AIDc_WR-C-GS MC3%; cds:All G total; cds:All A non-syn %; cds:ADAR_2Gen2_T-T-%; cds:3Gen2_A-C-C non-syn %; g:3Gen3_CA-C-C>T+G>A g %; g:ADARk_CW-A-A>G+T>C g %; nc:ADARb_W-A-Y A>G+T>C nc %; g:2Gen1_-C-T %; cds:Other MC3 C %; g:2Gen1_-C-T C>G+G>C g %; cds:ADAR_W-A-non-syn %; g:3Gen2_A-C-C C>A+G>T g %; g:ADAR_2Gen2_G-T-A>T+T>A %; cds:A3G_C-C-C>T at MC1%; cds:3Gen1_-C-GC MC2%; cds:3Gen2_G-C-T %; cds:A3F_T-C-G>C %; g:4Gen3_GG-C-G C>T+G>A g %; cds:A3Gb_-C-G G>A at MC2 motif %; cds:ADARb_W-A-Y MC2%; cds:All G %; g:A3F_T-C-Hits; cds:3Gen2_T-C-C MC1%; cds:A3B_T-C-W Ti %; cds:ADAR_3Gen1_-A-AT Ti %; cds:ADARh_W-A-S T>C %; cds:A3Gn_YYC-C-S C>T %; cds:A3Ge_SC-C-GS %; cds:2Gen2_A-C-MC3%; cds:ADAR_2Gen2_G-T-MC2%; cds:ADAR_3Gen3_CA-A-Ti %; cds:Primary Deaminase %; g:C>G+G>C %; cds:A3Bf_ST-C-G Ti %; cds:3Gen3_CT-C-MC3%; cds:A3Gi_SG-C-G non-syn %; cds:Other MC3%; cds:ADAR_3Gen1_-A-CA %; cds:A3F_T-C-C>A %; cds:2Gen1_-C-C C>T at MC1%; cds:A3Gc_C-C-GW C>T motif %; cds:AIDc_WR-C-GS %; g:ADAR_2Gen1_-T-T A>T+T>A %; cds:A3B_T-C-W MC1%; cds:ADAR_3Gen2_G-A-C non-syn %; cds:2Gen1_-C-C C>A %; cds:3Gen1_-C-GT G>A motif %; cds:A3Bj_RT-C-G Ti %; g:3Gen1_-C-TC C>T+G>A g %; g:C>A+G>T %; cds:3Gen2_A-C-C MC2%; cds:2Gen1_-C-C MC2%; g:3Gen2_G-C-T %; g:A3Bj_RT-C-G C>T+G>A g %; g:ADAR_W-A-A>G+T>C %; cds:3Gen3_AT-C-C:G %; cds:3Gen1_-C-TG G non-syn %; cds:Other G MC3 Ti/Tv %; cds:A3Gb_-C-G G>A MC2 Hits; cds:3Gen1_-C-TC C>T cds %; cds:2Gen1_-C-T MC3 non-syn %; cds:AIDb_WR-C-G G non-syn %; g:AIDc_WR-C-GS Hits; cds:3Gen2_T-C-C MC3%; cds:3Gen2_T-C-G Ti/Tv %; cds:A1_-C-A G>A at MC3 cds %; nc:A3G_C-C-C>T+G>A nc %; nc:2Gen2_A-C-C>T+G>A nc %; cds:3Gen3_TG-C-G Ti/Tv %; cds:3Gen1_-C-CA Ti %; cds:3Gen3_TG-C-G>A %; cds:3Gen3_CT-C-G non-syn %; cds:All C Ti/Tv %; cds:A3G_C-C-MC3%; cds:ADARc_SW-A-Y MC2%; and cds:3Gen3_GG-C-non-syn %, and related metrics thereto;
d) the cancer is sarcoma and the plurality of metrics comprises least or about 5 metrics selected from cds:Other MC3 C %; nc:ADARb_W-A-Y A>G+T>C nc %; cds:4Gen3_TT-C-T %; g:ADARk_CW-A-A>G+T>C g %; g:ADARn_-A-WA A>G+T>C %; cds:A3G_C-C-G>T %; cds:A3Gb_-C-G MC1%; nc:ADARb_W-A-Y %; cds:A3Ge_SC-C-GS %; cds:Primary Deaminase %; cds:ADAR_2Gen2_G-T-MC2%; g:4Gen3_GG-C-G C>T+G>A g %; cds:2Gen1_-C-C MC2%; cds:3Gen1_-C-GT G>A motif %; cds:A3Gn_YYC-C-S C>T %; cds:2Gen1_-C-C C>T at MC1%; cds:A3B_T-C-W MC3 non-syn %; cds:AIDd_WR-C-Y %; g:3Gen3_CA-C-C>T+G>A g %; cds:All A non-syn %; g:2Gen1_-C-T C>G+G>C g %; cds:ADARb_W-A-Y MC2%; cds:All G %; g:A3Bj_RT-C-G C>T+G>A g %; cds:A3Gn_YYC-C-S C>T at MC3 cds %; cds:A3B_T-C-W G non-syn %; cds:A3G_C-C-MC3%; cds:All G total; cds:CDS Variants; g:CG total; g:3Gen2_T-C-G C>T+G>A g %; cds:A3B_T-C-W MC1%; cds:ADAR_3Gen3_CA-A-Ti %; cds:AIDc_WR-C-GS %, and related metrics thereto;
e) the cancer is lung cancer and the plurality of metrics comprises least or about 5 metrics selected from cds:3Gen1_-C-CC C>T at MC1 motif %; cds:3Gen1_-C-CT C>T at MC2 cds %; cds:ADARp-A-WT A>G at MC2 cds %; cds:Other MC3 C %; cds:Other MC3%; cds:A3Gb_-C-G MC1%; g:3Gen1_-C-TC C>T+G>A g %; cds:ADAR_W-A-A>G at MC3%; cds:ADAR_W-A-non-syn %; cds:ADAR_3Gen3_AC-A-A>G cds %; cds:2Gen1_-C-C C>A %; cds:ADARf_SW-A-MC2%; g:ADAR_2Gen2_G-T-A>T+T>A %; cds:4Gen3_GC-C-A %; cds:A3Go_TC-C-G MC1 non-syn %; g:3Gen2_G-C-T %; cds:A3G_C-C-C>T at MC1%; cds:AIDc_WR-C-GS MC3%; cds:3Gen1_-C-GT G>A motif %; nc:2Gen1_-C-T C>A+G>T nc %; cds:ADARc_SW-A-Y MC2%; cds:ADARh_W-A-S T>C %; cds:2Gen1_-C-C C>T at MC1%; g:ADAR_2Gen1_-T-T A>T+T>A %; cds:AIDd_WR-C-Y C>A cds %; nc:A3G_C-C-C>T+G>A nc %; cds:A3Gc_C-C-GW C>T motif %; cds:ADAR_3Gen1_-A-AT Ti %; cds:3Gen3_CT-C-MC3%; cds:4Gen3_CT-C-C C>T at MC1%; cds:3Gen2_T-C-C MC1%; cds:A3G_C-C-G>T %; cds:3Gen1_-C-CA Ti %; cds:3Gen1_-C-TG G non-syn %; cds:3Gen2_A-C-C non-syn %; g:2Gen1_-C-T C>G+G>C g %; cds:All A non-syn %; cds:A3Gi_SG-C-G MC2%; cds:Primary Deaminase %; cds:4Gen3_TT-C-T %; g:A3Bj_RT-C-G C>T+G>A g %; cds:3Gen2_T-C-C MC3%; cds:4Gen3_TT-C-C %; cds:3Gen1_-C-CA Ti C:G %; cds:A1_-C-A G>A at MC3 cds %; cds:A3Gb_-C-G G>A at MC2 motif %; cds:3Gen3_CT-C-G non-syn %; cds:3Gen2_G-C-T C:G %; cds:A3Ge_SC-C-GS %; cds:3Gen3_TG-C-G>A %; g:C>A+G>T %; cds:4Gen3_CA-C-C %; cds:AIDd_WR-C-Y G>C %; cds:All G %; cds:3Gen3_TT-C-C>A at MC1 motif %; g:AIDh_WR-C-T C>A+G>T g %; g:4Gen3_GG-C-G C>T+G>A g %; cds:3Gen2_G-C-T C>A motif %; nc:ADARc_SW-A-Y A>G+T>C nc %; g:3Gen2_A-C-C C>A+G>T g %; cds:A3B_T-C-W Ti %; g:3Gen3_GA-C-C>A+G>T g %; cds:3Gen3_CT-C-C>T at MC1 motif %; cds:ADAR_3Gen1_-A-CC A>G cds %; cds:3Gen1_-C-TC C>T cds %; cds:4Gen3_CA-C-C MC1%; cds:3Gen2_G-C-T %; nc:2Gen2_A-C-C>T+G>A nc %; cds:3Gen2_A-C-C MC2%; cds:A3F_T-C-C>A %; cds:CDS Variants; cds:ADAR_3Gen3_CA-A-Ti %; cds:3Gen3_GG-C-non-syn %; cds:ADARb_W-A-Y MC2%; g:ADAR_W-A-A>G+T>C %; cds:3Gen3_AT-C-C:G %; cds:2Gen1_-C-C G>T at MC1%; cds:A3G_C-C-MC3%; cds:3Gen2_C-C-C MC3%; cds:A3B_T-C-W G>A motif %; cds:A3F_T-C-G>C %; cds:ADAR_2Gen2_G-T-MC2%; cds:3Gen1_-C-AG G Ti/Tv %; cds:A3Bj_RT-C-G Ti %; nc:ADARb_W-A-Y A>G+T>C nc %; cds:ADAR_2Gen2_T-T-%; g:2Gen1_-C-T %; cds:4Gen3_AC-C-T Ti/Tv %; cds:A3Gi_SG-C-G non-syn %; cds:A3Bf_ST-C-G Ti %; g:ADARk_CW-A-A>G+T>C g %; cds:3Gen1_-C-GC MC2%; g:3Gen3_CA-C-C>T+G>A g %; cds:2Gen2_A-C-MC3%; variants in VCF; cds:4Gen3_AG-C-T MC1 non-syn %; g:3Gen2_T-C-G C>T+G>A g %; cds:A3Gn_YYC-C-S C>T at MC3 cds %; cds:ADAR_3Gen1_-A-CA %; cds:4Gen3_TA-C-C non-syn %; cds:All C Ti/Tv %; cds:ADARc_SW-A-Y, and related metrics thereto; or
f) the cancer is skin cancer and the plurality of metrics comprises least or about 5 metrics selected from cds:4Gen3_AG-C-T MC1 non-syn %; cds:3Gen1_-C-CG G>A at MC3%; cds:4Gen3_AC-C-T Ti/Tv %; g:C>G+G>C %; cds:A3B_T-C-W MC3 non-syn %; cds:All A non-syn %; cds:3Gen3_AG-C-MC2%; cds:A3B_T-C-W MC1%; cds:ADAR_3Gen2_C-A-C T>G at MC3 cds %; cds:3Gen1_-C-TC C>T at MC3%; cds:4Gen3_GC-C-C C>T at MC2%; cds:All C Ti/Tv %; cds:A3Bj_RT-C-G Ti %; cds:AIDh_WR-C-T G>A at MC2 cds %; cds:4Gen3_TT-C-C %; cds:3Gen1_-C-CC C>T at MC1 motif %; cds:ADAR_2Gen2_T-T-%; cds:3Gen2_T-C-C MC1%; cds:All G %; cds:ADAR_W-A-A>G at MC3%; cds:A3G_C-C-MC3%; cds:Other MC3 C %; g:3Gen2_A-C-C C>A+G>T g %; cds:ADARc_SW-A-Y MC2%; cds:3Gen1_-C-CA Ti C:G %; cds:3Gen1_-C-TC C>T cds %; cds:3Gen2_C-C-C MC3%; cds:3Gen3_CT-C-C>T at MC1 motif %; g:ADAR_4Gen3_AG-A-G A>C+T>G %; cds:3Gen3_CT-C-G non-syn %; cds:3Gen2_A-C-C non-syn %; cds:2Gen2_A-C-MC3%; cds:3Gen2_A-C-C MC2%; g:3Gen1_-C-TC C>T+G>A g %; cds:3Gen2_T-C-T G>A at MC2%; cds:2Gen1_-C-C C>T at MC1%; cds:AIDb_WR-C-G G non-syn %; cds:A3Gb_-C-G MC1%; cds:2Gen1_-C-C C>A %; cds:A3Ge_SC-C-GS %; g:ADARn_-A-WA A>G+T>C %; g:ADAR_W-A-A>G+T>C %; g:ADAR_2Gen2_G-T-A>T+T>A %; g:AIDh_WR-C-T C>A+G>T g %; cds:4Gen3_TG-C-T Ti C:G %; cds:3Gen2_G-C-T C:G %; cds:3Gen2_T-C-C MC3%; nc:ADARb_W-A-Y %; cds:ADAR_3Gen2_G-A-C non-syn %; cds:ADAR_3Gen1_-A-AT Ti %; g:ADARk_CW-A-A>G+T>C g %; cds:3Gen1_-C-GC MC2%; cds:4Gen3_TA-C-C non-syn %; g:3Gen3_CA-C-C>T+G>A g %; cds:3Gen1_-C-AG G Ti/Tv %; cds:AIDc_WR-C-GS %; cds:A3Gn_YYC-C-S C>T at MC3 cds %; cds:2Gen1_-C-C MC2%; cds:3Gen3_GG-C-non-syn %; g:2Gen1_-C-T C>G+G>C g %; cds:A1_-C-A G>A at MC3 cds %; cds:A3G C-C-C>T at MC1%; nc:ADARc_SW-A-Y A>G+T>C nc %; cds:ADAR_W-A-T>C at MC2%; cds:A3Go_TC-C-G MC1 non-syn %; cds:3Gen3_AT-C-C:G %; cds:ADARh_W-A-S T>C %; cds:A3G_C-C-G>T %; cds:ADARf_SW-A-MC2%; cds:ADAR_W-A-non-syn %; cds:ADARp_-A-WT T>A motif %; cds:4Gen3_AG-C-T G>A at MC1 motif %; cds:ADAR_3Gen1_-A-CA %; cds:3Gen2_C-C-T MC3%; cds:3Gen1_-C-CT C>T at MC2 cds %; cds:A3B_T-C-W Ti %; g:2Gen1_-C-T %; cds:AIDc_WR-C-GS MC3%; cds:AIDe_WR-C-GW Hits; cds:AIDd_WR-C-Y C>A cds %; cds:ADARb_W-A-Y MC2%; cds:A3Gc_C-C-GW C>T motif %; cds:2Gen1_-C-C G>T at MC1%; cds:3Gen1_-C-CA Ti %; cds:Other G MC3 Ti/Tv %; cds:CDS Variants; cds:ADAR_3Gen1_-A-CC A>G cds %; cds:A3Gn_YYC-C-S C>T %; cds:A3Bf_ST-C-G Ti %; cds:2Gen2_G-C-Hits; cds:AIDd_WR-C-Y %; cds:A3F_T-C-G>C %; cds:4Gen3_CT-C-C C>T at MC1%; cds:AIDd_WR-C-Y G>C %; cds:A3Gi_SG-C-G MC2%; cds:Other MC3%; nc:2Gen1_-C-T C>A+G>T nc %; cds:3Gen2_G-C-T %; g:3Gen2_T-C-G C>T+G>A g %; cds:ADARc_SW-A-Y T>C cds %, and related metrics thereto.
